SEQUENCE LISTING (45 sequences)

SEQ ID NO: 1 - DNA, 1389 bp, Thiobacillus denitrificans
atggaccaatctgctagatacgctgacttgtccttgaaagaagaggacttgatcaagggtggtagacacatcttggttgcttacaagatgaagccaaagtccggttacggttacttggaagctgctgctcatttcgctgctgaatcttccactggaactaacgttgaggtttccactactgacgacttcactaagggtgttgacgctttggtttactatatcgacgaagcttccgaggacatgagaatcgcttacccattggagttgttcgacagaaacgttactgacggtagattcatgttggtttctttcttgactttggctatcggtaacaaccagggtatgggtgacattgagcacgctaagatgatcgacttctacgttcctgagagatgtatccagatgttcgacggtccagctactgacatttccaacttgtggagaatcttgggtagaccagttgttaacggtggttacattgctggtactatcatcaagccaaagttgggtcttagaccagagccatttgctaaggctgcttaccaattttggttgggtggtgacttcatcaagaacgacgagccacaaggtaaccaggttttctgtccattgaagaaggttttgccattggtttacgacgctatgaagagagctcaagacgacactggtcaggctaagttgttctccatgaacatcactgctgacgaccactacgagatgtgtgctagagctgattacgctttggaggttttcggtccagatgctgacaagttggctttcttggttgacggttacgttggtggtccaggtatggttactactgctaggagacaatacccaggtcagtacttgcactaccatagagctggtcatggtgctgttacttctccatctgctaagagaggttacactgctttcgttttggctaagatgtccagattgcaaggtgcttccggtatccacgttggtactatgggttacggtaagatggaaggtgaaggtgacgacaagattatcgcttacatgatcgagagagatgagtgtcagggtccagtttacttccaaaagtggtacggtatgaagccaactactccaatcatctccggtggtatgaacgctttgagattgccaggtttcttcgagaacttgggtcacggtaacgttatcaacactgctggtggtggttcctacggtcacattgattctccagctgctggtgctatttccttgagacaatcttacgagtgttggaagcaaggtgctgacccaatcgaattcgctaaagagcacaaagagttcgctagagctttcgagtctttcccaaaggacgctgataagttgttcccaggttggagagaaaagttgggtgttcacaagtccaagttgtaa

SEQ ID NO: 2 - DNA, 1215 bp, Spinacia oleracea
atggctgtctgtactgtttacactatcccaactactactcacttgggttcctcattcaaccagaacaacaagcaggttttcttcaactacaagagatcctcctcctccaacaacactttgttcactactagaccatcctacgttatcacttgttcccagcagcagactatcgttatcggtttggctgctgattccggttgtggtaagtccacttttatgagaagattgacttccgttttcggtggtgctgctgaaccaccaaaaggtggtaacccagactccaacactttgatctccgacactactactgttatctgtttggacgacttccactccttggacagaaacggtagaaaggttgagaaggttactgctttggacccaaaggctaacgacttcgacttgatgtacgagcaggttaaggctttgaaagagggtaaggctgttgacaagccaatctacaaccacgtttccggtttgttggacccaccagagttgattcagccaccaaagatcttggttatcgagggattgcacccaatgtacgacgctagagttagagagttgttggacttctccatctacttggacatctccaacgaggttaagttcgcttggaagatccagagagacatgaaggaaagaggtcactccttggagtccatcaaggcttctattgagtccagaaagccagacttcgacgcttacattgacccacaaaagcaacacgctgacgttgttatcgaggttttgccaactgagttgatcccagatgacgacgagggtaaggttttgagagttagaatgatccagaaagagggtgttaagttcttcaacccagtttacttgttcgacgagggttctactatctcctggatcccatgtggtagaaagttgacttgttcctacccaggtatcaagttctcctacggtccagacactttctacggtaacgaggttactgttgttgagatggacggtatgttcgacagattggacgagttgatctacgttgagtcccacttgtctaacttgtccactaagttctacggtgaggttactcagcagatgttgaagcaccaaaacttcccaggttccaacaacggtactggtttcttccagactatcatcggtttgaagatcagagacttgttcgagcagttggttgcttccagatctactgctactgctacagctgctaaggcttccaagttg

SEQ ID NO: 3 - DNA, 1260 bp, Ogataea polymorpha
atgtctcttgctaacaagctatccgtcaaggaccttcaattcacaggtaaaagagtgttcatcagagtggacttcaatgttcctcttgatggggacaagatcaccaacaatcagagaattgttgctgccttgccaaccatcaagtacgttttggatcaaaagccaaaggttgtcgttttggcttcccatttgggtagaccaaacggtgaggtgaacaagaaattcaccttgaagcctgttgctggcgaattggagtctttgctgggtaagaaggtcactttcttgtcggactgtgttggccctgaggtcgagtctgctgtcaacagtgctaccgacggggccgtaattctattggagaacctcagattccacattgaagaagagggatcgaagaaaacgccagagggaaaggtcaaggcttcgaaggaggacgttgagaagtttagaaaacaattgaccgccttggcggacgtctacgtcaacgacgctttcggtaccgcccacagagcccactcgtccatggttggctttgagctcaacgagagagccgctggtttcctgatggccaaggagctggagtacttttctaaggctttggagaacccagttagaccattcctggctattctgggaggtgccaaggtgtccgacaagatccaattgatcgataacttactcgacaaggtcgacatcctgatcatcggcggtggtatggccttcactttcaacaagattgtcaacaacatgaacattggaaaatccctatttgacaaggacggtgcagagatcgttcctaaactgatcgagaaggccaagaagaacggcgttgaggtcatccttcctgttgactttgtcactgccgacagcttctctccagacgccaagaccggctacgctactatggaggaaggcattcctgacgactggcaaggactggacgctggcgagaagtcccgcaaactttacgccgacgcaattgccaaggccaagaccattgtttggaacggtccaccgggtgtctttgagttcgagaagtttgccgacggaaccaagtccatgctcgaggcctgtgtcaagagtgcccaggctggaaacaccgtcatcattggaggtggtgacactgccaccgttgccaagaagtttggtggagcagacaagttgtctcacgtttctactggtggaggtgcttctctggagcttttggagggtaaggagttgccaggtgtggttgctttgggaaacaaagcatctaagttgtaa

SEQ ID NO: 4 - DNA, 1017 bp, Ogataea polymorpha
atgaccgcaaacgttggaattaatggatttggaagaattggtagactggtgttgagaattgccttgagcagagacgacatcaacgtcattgccatcaatgatccattcattgctcctgattacgccgcttacatgttcaagtacgactctacacacggaaagttcaagggaactgttacccacgagggtaagtacttggtcattgacggcaagaagattgaggttttccaagagagagatccagcaaacatcccatggggtaaggagggcgtcgactacgttctggactctaccggagttttcaccaccttggagggcgctcaaaagcacattgatgctggtgccaagaaggtcatcatcactgctccatctaaggatgctccaatgttcgttatgggtgtgaaccacgaggagtacactccagacatcaagattctgtctaacgcttcttgtaccaccaactgcctggcaccaatggccaaggttatcaacgacacctacggaattgaggaaggtttgatgaccactattcactccatcaccgctactcagaaaactgttgacggtccatcccacaaggactggagaggtggtagaaccgcttccggtaacatcatcccatcctctactggtgctgccaaggctgtcggaaaggtccttcctgcattggccggtaagctcactggtatgtccatgagagtcccaaccaccgatgtttctgttgttgacttgaccgtcaacctcaagaagccaaccacctacgaggacatttgcgccaccatgaagaaggctgctgagggcccattggctggaatccttggatacaccgacgaggcagtcgtttcgtccgacttcgtgaccgacaccagatcctctgtctttgacgccaaggccggtatcctgctgaccccaaccttcgtcaagctcgtttcctggtatgacaatgagtacggctactctaccagagttgttgacttgcttgagcacgttgctaaggtttccgcctctaagttgtaa

SEQ ID NO: 5 - DNA, 846 bp, Ogataea parapolymorpha
atggtacgttccgcattaacactggtccatgcgatctctggccatttgagtcccgctgaccactcccgatattcaaaaactaactctttaccacaggctagacaattcttcgttggtggcaactttaaaatgaacggttcaaaggactcgattcaatcgattgtccagaacctcaattccgcctctcttccttccaatgtccaggtcgtgattgctcctccagctccataccttttctgggccgctgagcacaacaagcaaccaaccgtggaggtggctgcccagaactgttacgacaaggcttcgggtgcctacaccggagaagtttctgcagtggctctcaaggacttgggcatcccatgggtgattttgggtcactctgaaagaagaatcatcttcaaggagtctgacgaattgattgcttccaaaactaaatttgcgttggactctggactcaaggtgatcttgtgtattggagagactcttgaggagaaaaaagccggcaagaccgtggaggtgtgtaagagacagcttcaagccgtgcttgacgtcgtgtccgactggagcaacattgtgatcgcttacgagccagtttgggccatcggtacgggtctggctgccactgctgatgacgcacaggaaatccacaaggacatcagaaacttcttgagcacggctattggctcccaggctgagtctgttagaatcttgtacggaggctctgtgagtggcaagaacgctaaagactttagaggcaaggatgatgtcgatggattcttggttggaggtgcttctctaaagccggaatttgttgacattatcaaatccagagagtctaagttgtaa

SEQ ID NO: 6 - DNA, 2040 bp, Ogataea parapolymorpha
atgtcccaagatatccaaacaaaagccattaatacaatcagagtcctggctgccgacatcgtcgccaaggccaactccgggcaccctggtgccccaatgggaatggccccagccgcccacgccgtctacactcagatgaaattcaacccaaagaacccacactggatcaacagagatagatttgtcctttcgaacggtcacgcatgtgctctcttgtacacattgctgcacctgttcggctatgacctcaccatggacgacctcaagtcgttcagacagatcaactctaagactccgggccaccctgaagtgggcattccaggtgtcgaggtcacaaccggtccgcttggtcagggtatctccaatgctgttggtttggccatcgctcaggctaactttgctgccacctacaacaagccaggctacactctctccgacaactacacctactgtttctttggtgacggatgtatgatggagggtgttgcctccgaggccatgtctctcgccggccacctgcaattgggcaacctgatcgctttctacgatgacaacaagatctccatcgacggaagcactgaggttgctttcaccgaggacgtttgcggaaggctcgagaagtacggatgggacatcttcgaggtccctgacgctgataccgacgttgccgccatttccgaggccattgccaaggccaagaaaaccaacaagccttcttgtatcagaatcagaaccacgattggttacggttcgctcaacgctggctcccactccgtccacggtgctcctctcaagaaagacgacattattcaattgaaggagaaatggggcttcgacccagaaaagtcatttgttgttccacaagaggtttacgactactaccacaagatttctgaggccggtgctgctgccgaggccgagtggaacaagctctttgaggcctaccagaaggagttccctaaggagggtgcagagcttgccagaaggctcaggggcgagcttccagaaggctggcaaaacgtcctcccaacttaccagccaggcgacgctgctgttgcttccagaaagctttctgaggtttgcttgggtaagttgcaagctgttcttccagaattggttggtggttctgccgatttgaccccatccaacttgaccagatggtctggtgccattgacttccaacctgagtccactggtctgggtgactactccggtaagtacctcagattcggtgtgagagaacacggtatgggcgccatcatcaacggtatttctgcttacggtgccaactacaagtctttcggtgctactttcctcaactttgtgtcgtacgcctctggtgccctgagattgtctgctctctcgcaccacccagtcatctgggttgccacccacgactctattggtcttggtgaggacggaccaacccaccagcctattgagactcttgctcacttcagagccattccaaacctgatggtctggaggccagctgacggtaacgaaacctctgctgcttacatcaaggccatctcgagcaccaagactccatctgtgctggctctttctagacaaaacttgcctcaactgcctggctcttctattgagaaggccttgaagggtggttacactgttcacgaggttgacaacgccaagctgattttggttgctaccggttccgaggtttctctgtccatcgatgctgccaagctgcttactgagaagggtatcccaactgctgttgtttccatcccagacttcttcactttcgaccagcagccagccgactacaagctgtctgtgttgccagacggtgttccaatcttgtctgtcgaggtgatggccacctctggatgggccaagtacgcccacgcccaattcggcctcaacagatttggtgcctccggcaagactgccgacgtgtacaagttcttcgacttcaccccagagggtgttgccaagagaggtgagcagacctacgaattcttcaagggtaagaacctgatttctccattgcacactcctttctctaagttgtaa

SEQ ID NO: 7 - DNA, 1647 bp, Escherichia coli
atggctgctaaggatgttaagttcggtaacgacgctagagttaagatgttgagaggtgttaacgttttggctgacgctgttaaggttactttgggtccaaagggtagaaacgttgttttggacaagtccttcggtgctccaactatcactaaggacggtgtttctgttgctagagagatcgagttggaggacaagttcgaaaacatgggtgctcagatggttaaggaagttgcttccaaggctaacgatgctgctggtgatggtactactactgctactgttttggctcaggctatcatcactgagggtttgaaggctgttgctgctggtatgaacccaatggacttgaagagaggtatcgacaaggctgttacagcagctgttgaagagttgaaggctttgtccgttccatgttctgactccaaggctattgctcaggttggtactatttccgctaactccgacgagactgttggaaagttgattgctgaggctatggacaaggttggtaaagagggtgttatcactgttgaggacggtactggattgcaagacgagttggatgttgttgagggtatgcagttcgacagaggttacttgtccccatacttcatcaacaagccagagactggtgctgttgaattggagtccccattcatcttgttggctgacaagaagatctccaacatcagagagatgttgccagttttggaagctgttgctaaggctggtaagcctttgttgattatcgctgaggacgttgagggtgaggctttggctactttggttgttaacactatgagaggtatcgttaaggttgctgctgttaaggctccaggtttcggtgatagaagaaaggctatgttgcaggacattgctactttgactggtggtactgttatctccgaagagatcggtatggaattggagaaggctactttggaggacttgggtcaggctaagagagttgttatcaacaaggacactactactatcatcgacggtgttggtgaagaggctgctattcaaggtagagttgctcagatcagacagcagatcgaagaagctacttccgactacgacagagagaagttgcaagagagagttgctaagttggctggtggtgttgctgttatcaaggttggtgctgctactgaggttgagatgaaggaaaagaaagctagagttgaggacgctctgcacgctactagagctgctgttgaagagggtgttgttgctggtggtggtgttgctttgattagagttgcttctaaattggctgacttgagaggtcagaacgaggaccagaacgttggtatcaaggttgctttgagagctatggaagctccattgagacagatcgttttgaactgtggtgaggaaccatccgttgttgctaacactgttaagggtggtgacggtaactacggttacaacgctgctactgaagagtacggtaacatgatcgacatgggtatcttggacccaactaaggttactagatccgctctgcaatacgctgcttccgttgctggtttgatgatcactactgagtgtatggttactgacttgccaaagaacgacgctgctgatttgggtgctgctggtggtatgggtggaatgggaggtatgggtggtatgatgtaa

SEQ ID NO: 8 - DNA, 291 bp, Escherichia coli
atgaacatcagaccattgcacgacagagttatcgttaagagaaaagaggttgagactaagtccgctggtggtatcgttttgactggttctgctgctgctaagtccactagaggtgaagttttggctgttggtaacggtagaatcttggagaacggtgaggttaagccattggacgttaaggttggtgacatcgttatcttcaacgacggttacggtgttaagtccgagaagatcgacaacgaagaggttttgatcatgtccgagtccgacatcttggctatcgttgaagct

SEQ ID NO: 9 - DNA, 20 bp, Pichia pastoris
ctaggatatcaaactcttcg

SEQ ID NO: 10 - DNA, 20 bp, Pichia pastoris
tggagaataatcgaacaaaa

SEQ ID NO: 11 - DNA, 20 bp, Pichia pastoris
cgacaaactataagtagatt

SEQ ID NO: 12 - protein, 9 aa, Artificial Sequence
  Signal sequence
  MISC_FEATURE (1)..(1): X is any of R or K
  MISC_FEATURE (2)..(2): X is any of L, V or I
  MISC_FEATURE (3)..(3): X is any amino acid
  MISC_FEATURE (4)..(7): X is no or any amino acid
  MISC_FEATURE (8)..(8): X is any of H or Q
  MISC_FEATURE (9)..(9): X is any of L or A
XXXXXXXXX

SEQ ID NO: 13 - protein, 9 aa, Artificial Sequence
  Signal sequence
  MISC_FEATURE (3)..(7): X is any amino acid
RLXXXXXHL

SEQ ID NO: 14 - protein, 9 aa, Artificial Sequence
  Signal sequence
  MISC_FEATURE (3)..(7): X is any amino acid
RLXXXXXHA

SEQ ID NO: 15 - protein, 9 aa, Artificial Sequence
  Signal sequence
  MISC_FEATURE (3)..(7): X is any amino acid
RLXXXXXQL

SEQ ID NO: 16 - protein, 9 aa, Artificial Sequence
  Signal sequence
  MISC_FEATURE (3)..(7): X is any amino acid
RLXXXXXQA

SEQ ID NO: 17 - protein, 9 aa, Artificial Sequence
  Signal sequence
  MISC_FEATURE (3)..(7): X is any amino acid
RVXXXXXHV

SEQ ID NO: 18 - protein, 9 aa, Artificial Sequence
  Signal sequence
  MISC_FEATURE (3)..(7): X is any amino acid
RVXXXXXHA

SEQ ID NO: 19 - protein, 9 aa, Artificial Sequence
  Signal sequence
  MISC_FEATURE (3)..(7): X is any amino acid
RVXXXXXQV

SEQ ID NO: 20 - protein, 9 aa, Artificial Sequence
  Signal sequence
  MISC_FEATURE (3)..(7): X is any amino acid
RVXXXXXQA

SEQ ID NO: 21 - protein, 9 aa, Artificial Sequence
  Signal sequence
  MISC_FEATURE (3)..(7): X is any amino acid
RIXXXXXHI

SEQ ID NO: 22 - protein, 9 aa, Artificial Sequence
  Signal sequence
  MISC_FEATURE (3)..(7): X is any amino acid
RIXXXXXHA

SEQ ID NO: 23 - protein, 9 aa, Artificial Sequence
  Signal sequence
  MISC_FEATURE (3)..(7): X is any amino acid
RIXXXXXQI

SEQ ID NO: 24 - protein, 9 aa, Artificial Sequence
  Signal sequence
  MISC_FEATURE (3)..(7): X is any amino acid
RIXXXXXQA

SEQ ID NO: 25 - protein, 9 aa, Artificial Sequence
  Signal sequence
  MISC_FEATURE (3)..(7): X is any amino acid
KLXXXXXHL

SEQ ID NO: 26 - protein, 9 aa, Artificial Sequence
  Signal sequence
  MISC_FEATURE (3)..(7): X is any amino acid
KLXXXXXHA

SEQ ID NO: 27 - protein, 9 aa, Artificial Sequence
  Signal sequence
  MISC_FEATURE (3)..(7): X is any amino acid
KLXXXXXQL

SEQ ID NO: 28 - protein, 9 aa, Artificial Sequence
  Signal sequence
  MISC_FEATURE (3)..(7): X is any amino acid
KLXXXXXQA

SEQ ID NO: 29 - protein, 9 aa, Artificial Sequence
  Signal sequence
  MISC_FEATURE (3)..(7): X is any amino acid
KVXXXXXHV

SEQ ID NO: 30 - protein, 9 aa, Artificial Sequence
  Signal sequence
  MISC_FEATURE (3)..(7): X is any amino acid
KVXXXXXHA

SEQ ID NO: 31 - protein, 9 aa, Artificial Sequence
  Signal sequence
  MISC_FEATURE (3)..(7): X is any amino acid
KVXXXXXQV

SEQ ID NO: 32 - protein, 9 aa, Artificial Sequence
  Signal sequence
  MISC_FEATURE (3)..(7): X is any amino acid
KVXXXXXQA

SEQ ID NO: 33 - protein, 9 aa, Artificial Sequence
  Signal sequence
  MISC_FEATURE (3)..(7): X is any amino acid
KIXXXXXHI

SEQ ID NO: 34 - protein, 9 aa, Artificial Sequence
  Signal sequence
  MISC_FEATURE (3)..(7): X is any amino acid
KIXXXXXHA

SEQ ID NO: 35 - protein, 9 aa, Artificial Sequence
  Signal sequence
  MISC_FEATURE (3)..(7): X is any amino acid
KIXXXXXQI

SEQ ID NO: 36 - protein, 9 aa, Artificial Sequence
  Signal sequence
  MISC_FEATURE (3)..(7): X is any amino acid
KIXXXXXQA

SEQ ID NO: 37 - DNA, 1377 bp, Thiobacillus denitrificans
atggaccaatctgctagatacgctgacttgtccttgaaagaagaggacttgatcaagggtggtagacacatcttggttgcttacaagatgaagccaaagtccggttacggttacttggaagctgctgctcatttcgctgctgaatcttccactggaactaacgttgaggtttccactactgacgacttcactaagggtgttgacgctttggtttactatatcgacgaagcttccgaggacatgagaatcgcttacccattggagttgttcgacagaaacgttactgacggtagattcatgttggtttctttcttgactttggctatcggtaacaaccagggtatgggtgacattgagcacgctaagatgatcgacttctacgttcctgagagatgtatccagatgttcgacggtccagctactgacatttccaacttgtggagaatcttgggtagaccagttgttaacggtggttacattgctggtactatcatcaagccaaagttgggtcttagaccagagccatttgctaaggctgcttaccaattttggttgggtggtgacttcatcaagaacgacgagccacaaggtaaccaggttttctgtccattgaagaaggttttgccattggtttacgacgctatgaagagagctcaagacgacactggtcaggctaagttgttctccatgaacatcactgctgacgaccactacgagatgtgtgctagagctgattacgctttggaggttttcggtccagatgctgacaagttggctttcttggttgacggttacgttggtggtccaggtatggttactactgctaggagacaatacccaggtcagtacttgcactaccatagagctggtcatggtgctgttacttctccatctgctaagagaggttacactgctttcgttttggctaagatgtccagattgcaaggtgcttccggtatccacgttggtactatgggttacggtaagatggaaggtgaaggtgacgacaagattatcgcttacatgatcgagagagatgagtgtcagggtccagtttacttccaaaagtggtacggtatgaagccaactactccaatcatctccggtggtatgaacgctttgagattgccaggtttcttcgagaacttgggtcacggtaacgttatcaacactgctggtggtggttcctacggtcacattgattctccagctgctggtgctatttccttgagacaatcttacgagtgttggaagcaaggtgctgacccaatcgaattcgctaaagagcacaaagagttcgctagagctttcgagtctttcccaaaggacgctgataagttgttcccaggttggagagaaaagttgggtgttcacaag

SEQ ID NO: 38 - DNA, 1206 bp, Spinacia oleracea
atggctgtctgtactgtttacactatcccaactactactcacttgggttcctcattcaaccagaacaacaagcaggttttcttcaactacaagagatcctcctcctccaacaacactttgttcactactagaccatcctacgttatcacttgttcccagcagcagactatcgttatcggtttggctgctgattccggttgtggtaagtccacttttatgagaagattgacttccgttttcggtggtgctgctgaaccaccaaaaggtggtaacccagactccaacactttgatctccgacactactactgttatctgtttggacgacttccactccttggacagaaacggtagaaaggttgagaaggttactgctttggacccaaaggctaacgacttcgacttgatgtacgagcaggttaaggctttgaaagagggtaaggctgttgacaagccaatctacaaccacgtttccggtttgttggacccaccagagttgattcagccaccaaagatcttggttatcgagggattgcacccaatgtacgacgctagagttagagagttgttggacttctccatctacttggacatctccaacgaggttaagttcgcttggaagatccagagagacatgaaggaaagaggtcactccttggagtccatcaaggcttctattgagtccagaaagccagacttcgacgcttacattgacccacaaaagcaacacgctgacgttgttatcgaggttttgccaactgagttgatcccagatgacgacgagggtaaggttttgagagttagaatgatccagaaagagggtgttaagttcttcaacccagtttacttgttcgacgagggttctactatctcctggatcccatgtggtagaaagttgacttgttcctacccaggtatcaagttctcctacggtccagacactttctacggtaacgaggttactgttgttgagatggacggtatgttcgacagattggacgagttgatctacgttgagtcccacttgtctaacttgtccactaagttctacggtgaggttactcagcagatgttgaagcaccaaaacttcccaggttccaacaacggtactggtttcttccagactatcatcggtttgaagatcagagacttgttcgagcagttggttgcttccagatctactgctactgctacagctgctaaggct

SEQ ID NO: 39 - DNA, 1248 bp, Ogataea polymorpha
atgtctcttgctaacaagctatccgtcaaggaccttcaattcacaggtaaaagagtgttcatcagagtggacttcaatgttcctcttgatggggacaagatcaccaacaatcagagaattgttgctgccttgccaaccatcaagtacgttttggatcaaaagccaaaggttgtcgttttggcttcccatttgggtagaccaaacggtgaggtgaacaagaaattcaccttgaagcctgttgctggcgaattggagtctttgctgggtaagaaggtcactttcttgtcggactgtgttggccctgaggtcgagtctgctgtcaacagtgctaccgacggggccgtaattctattggagaacctcagattccacattgaagaagagggatcgaagaaaacgccagagggaaaggtcaaggcttcgaaggaggacgttgagaagtttagaaaacaattgaccgccttggcggacgtctacgtcaacgacgctttcggtaccgcccacagagcccactcgtccatggttggctttgagctcaacgagagagccgctggtttcctgatggccaaggagctggagtacttttctaaggctttggagaacccagttagaccattcctggctattctgggaggtgccaaggtgtccgacaagatccaattgatcgataacttactcgacaaggtcgacatcctgatcatcggcggtggtatggccttcactttcaacaagattgtcaacaacatgaacattggaaaatccctatttgacaaggacggtgcagagatcgttcctaaactgatcgagaaggccaagaagaacggcgttgaggtcatccttcctgttgactttgtcactgccgacagcttctctccagacgccaagaccggctacgctactatggaggaaggcattcctgacgactggcaaggactggacgctggcgagaagtcccgcaaactttacgccgacgcaattgccaaggccaagaccattgtttggaacggtccaccgggtgtctttgagttcgagaagtttgccgacggaaccaagtccatgctcgaggcctgtgtcaagagtgcccaggctggaaacaccgtcatcattggaggtggtgacactgccaccgttgccaagaagtttggtggagcagacaagttgtctcacgtttctactggtggaggtgcttctctggagcttttggagggtaaggagttgccaggtgtggttgctttgggaaacaaagca

SEQ ID NO: 40 - DNA, 1005 bp, Ogataea polymorpha
atgaccgcaaacgttggaattaatggatttggaagaattggtagactggtgttgagaattgccttgagcagagacgacatcaacgtcattgccatcaatgatccattcattgctcctgattacgccgcttacatgttcaagtacgactctacacacggaaagttcaagggaactgttacccacgagggtaagtacttggtcattgacggcaagaagattgaggttttccaagagagagatccagcaaacatcccatggggtaaggagggcgtcgactacgttctggactctaccggagttttcaccaccttggagggcgctcaaaagcacattgatgctggtgccaagaaggtcatcatcactgctccatctaaggatgctccaatgttcgttatgggtgtgaaccacgaggagtacactccagacatcaagattctgtctaacgcttcttgtaccaccaactgcctggcaccaatggccaaggttatcaacgacacctacggaattgaggaaggtttgatgaccactattcactccatcaccgctactcagaaaactgttgacggtccatcccacaaggactggagaggtggtagaaccgcttccggtaacatcatcccatcctctactggtgctgccaaggctgtcggaaaggtccttcctgcattggccggtaagctcactggtatgtccatgagagtcccaaccaccgatgtttctgttgttgacttgaccgtcaacctcaagaagccaaccacctacgaggacatttgcgccaccatgaagaaggctgctgagggcccattggctggaatccttggatacaccgacgaggcagtcgtttcgtccgacttcgtgaccgacaccagatcctctgtctttgacgccaaggccggtatcctgctgaccccaaccttcgtcaagctcgtttcctggtatgacaatgagtacggctactctaccagagttgttgacttgcttgagcacgttgctaaggtttccgcc

SEQ ID NO: 41 - DNA, 834 bp, Ogataea parapolymorpha
atggtacgttccgcattaacactggtccatgcgatctctggccatttgagtcccgctgaccactcccgatattcaaaaactaactctttaccacaggctagacaattcttcgttggtggcaactttaaaatgaacggttcaaaggactcgattcaatcgattgtccagaacctcaattccgcctctcttccttccaatgtccaggtcgtgattgctcctccagctccataccttttctgggccgctgagcacaacaagcaaccaaccgtggaggtggctgcccagaactgttacgacaaggcttcgggtgcctacaccggagaagtttctgcagtggctctcaaggacttgggcatcccatgggtgattttgggtcactctgaaagaagaatcatcttcaaggagtctgacgaattgattgcttccaaaactaaatttgcgttggactctggactcaaggtgatcttgtgtattggagagactcttgaggagaaaaaagccggcaagaccgtggaggtgtgtaagagacagcttcaagccgtgcttgacgtcgtgtccgactggagcaacattgtgatcgcttacgagccagtttgggccatcggtacgggtctggctgccactgctgatgacgcacaggaaatccacaaggacatcagaaacttcttgagcacggctattggctcccaggctgagtctgttagaatcttgtacggaggctctgtgagtggcaagaacgctaaagactttagaggcaaggatgatgtcgatggattcttggttggaggtgcttctctaaagccggaatttgttgacattatcaaatccagagag

SEQ ID NO: 42 - DNA, 2028 bp, Ogataea parapolymorpha
atgtcccaagatatccaaacaaaagccattaatacaatcagagtcctggctgccgacatcgtcgccaaggccaactccgggcaccctggtgccccaatgggaatggccccagccgcccacgccgtctacactcagatgaaattcaacccaaagaacccacactggatcaacagagatagatttgtcctttcgaacggtcacgcatgtgctctcttgtacacattgctgcacctgttcggctatgacctcaccatggacgacctcaagtcgttcagacagatcaactctaagactccgggccaccctgaagtgggcattccaggtgtcgaggtcacaaccggtccgcttggtcagggtatctccaatgctgttggtttggccatcgctcaggctaactttgctgccacctacaacaagccaggctacactctctccgacaactacacctactgtttctttggtgacggatgtatgatggagggtgttgcctccgaggccatgtctctcgccggccacctgcaattgggcaacctgatcgctttctacgatgacaacaagatctccatcgacggaagcactgaggttgctttcaccgaggacgtttgcggaaggctcgagaagtacggatgggacatcttcgaggtccctgacgctgataccgacgttgccgccatttccgaggccattgccaaggccaagaaaaccaacaagccttcttgtatcagaatcagaaccacgattggttacggttcgctcaacgctggctcccactccgtccacggtgctcctctcaagaaagacgacattattcaattgaaggagaaatggggcttcgacccagaaaagtcatttgttgttccacaagaggtttacgactactaccacaagatttctgaggccggtgctgctgccgaggccgagtggaacaagctctttgaggcctaccagaaggagttccctaaggagggtgcagagcttgccagaaggctcaggggcgagcttccagaaggctggcaaaacgtcctcccaacttaccagccaggcgacgctgctgttgcttccagaaagctttctgaggtttgcttgggtaagttgcaagctgttcttccagaattggttggtggttctgccgatttgaccccatccaacttgaccagatggtctggtgccattgacttccaacctgagtccactggtctgggtgactactccggtaagtacctcagattcggtgtgagagaacacggtatgggcgccatcatcaacggtatttctgcttacggtgccaactacaagtctttcggtgctactttcctcaactttgtgtcgtacgcctctggtgccctgagattgtctgctctctcgcaccacccagtcatctgggttgccacccacgactctattggtcttggtgaggacggaccaacccaccagcctattgagactcttgctcacttcagagccattccaaacctgatggtctggaggccagctgacggtaacgaaacctctgctgcttacatcaaggccatctcgagcaccaagactccatctgtgctggctctttctagacaaaacttgcctcaactgcctggctcttctattgagaaggccttgaagggtggttacactgttcacgaggttgacaacgccaagctgattttggttgctaccggttccgaggtttctctgtccatcgatgctgccaagctgcttactgagaagggtatcccaactgctgttgtttccatcccagacttcttcactttcgaccagcagccagccgactacaagctgtctgtgttgccagacggtgttccaatcttgtctgtcgaggtgatggccacctctggatgggccaagtacgcccacgcccaattcggcctcaacagatttggtgcctccggcaagactgccgacgtgtacaagttcttcgacttcaccccagagggtgttgccaagagaggtgagcagacctacgaattcttcaagggtaagaacctgatttctccattgcacactcctttc

SEQ ID NO: 43 - DNA, 1644 bp, Escherichia coli
atggctgctaaggatgttaagttcggtaacgacgctagagttaagatgttgagaggtgttaacgttttggctgacgctgttaaggttactttgggtccaaagggtagaaacgttgttttggacaagtccttcggtgctccaactatcactaaggacggtgtttctgttgctagagagatcgagttggaggacaagttcgaaaacatgggtgctcagatggttaaggaagttgcttccaaggctaacgatgctgctggtgatggtactactactgctactgttttggctcaggctatcatcactgagggtttgaaggctgttgctgctggtatgaacccaatggacttgaagagaggtatcgacaaggctgttacagcagctgttgaagagttgaaggctttgtccgttccatgttctgactccaaggctattgctcaggttggtactatttccgctaactccgacgagactgttggaaagttgattgctgaggctatggacaaggttggtaaagagggtgttatcactgttgaggacggtactggattgcaagacgagttggatgttgttgagggtatgcagttcgacagaggttacttgtccccatacttcatcaacaagccagagactggtgctgttgaattggagtccccattcatcttgttggctgacaagaagatctccaacatcagagagatgttgccagttttggaagctgttgctaaggctggtaagcctttgttgattatcgctgaggacgttgagggtgaggctttggctactttggttgttaacactatgagaggtatcgttaaggttgctgctgttaaggctccaggtttcggtgatagaagaaaggctatgttgcaggacattgctactttgactggtggtactgttatctccgaagagatcggtatggaattggagaaggctactttggaggacttgggtcaggctaagagagttgttatcaacaaggacactactactatcatcgacggtgttggtgaagaggctgctattcaaggtagagttgctcagatcagacagcagatcgaagaagctacttccgactacgacagagagaagttgcaagagagagttgctaagttggctggtggtgttgctgttatcaaggttggtgctgctactgaggttgagatgaaggaaaagaaagctagagttgaggacgctctgcacgctactagagctgctgttgaagagggtgttgttgctggtggtggtgttgctttgattagagttgcttctaaattggctgacttgagaggtcagaacgaggaccagaacgttggtatcaaggttgctttgagagctatggaagctccattgagacagatcgttttgaactgtggtgaggaaccatccgttgttgctaacactgttaagggtggtgacggtaactacggttacaacgctgctactgaagagtacggtaacatgatcgacatgggtatcttggacccaactaaggttactagatccgctctgcaatacgctgcttccgttgctggtttgatgatcactactgagtgtatggttactgacttgccaaagaacgacgctgctgatttgggtgctgctggtggtatgggtggaatgggaggtatgggtggtatgatg

SEQ ID NO: 44 - DNA, 9 bp, Artificial Sequence
  PTS coding sequence
tccaagttg

SEQ ID NO: 45 - DNA, 9 bp, Artificial Sequence
  PTS coding sequence
tctaagttg

(19) United States
(12) Patent Application Publication
(10) Pub. No.: US 2020/0181629 A1
(43) Pub. Date: Jun. 11, 2020

(54) YEAST EXPRESSING A SYNTHETIC CALVIN CYCLE

(71) Applicant: Universität für Bodenkultur Wien, Vienna (AT)

(72) Inventors: Diethard MATTANOVICH, Vienna (AT); Michael SAUER, Vienna (AT); Matthias STEIGER, Vienna (AT); Thomas GASSLER, Vienna (AT); Brigitte GASSER, Vienna (AT)

(21) Appl. No.: 16/617,716

(22) PCT Filed: May 30, 2018
(86) PCT No.: PCT/EP2018/064158

§ 371 Date: Nov. 27, 2019

(30) Foreign Application Priority Data
May 31, 2017 (EP) 17173655.6
Feb. 26, 2018 (EP) 18158536.5

(51) Int. Cl. C12N 15/81; C12P 7/46; C12N 9/48; C12N 9/10; C12N 9/88; C07K 14/245; C12N 9/12; C12N 9/02; C12P 7/56; C12N 1/16; C12N 9/90; C07K 14/765

(52) U.S. Cl. CPC C12N 15/815; C12Y 503/01001; C12Y 401/01039; C12Y 207/02003; C12Y 202/01001; C12N 9/485; C12N 9/1022; C12N 9/88; C07K 14/245; C12N 9/1294; C12N 9/0008; C12N 9/1217; C12P 7/56; C12N 1/16; C12Y 102/01012; C12Y 207/01019; C12Y 304/17002; C12N 9/90; C07K 14/765; C12P 7/46

(57) ABSTRACT

A yeast comprising a nucleotide sequence expression system expressing a synthetic Calvin cycle comprising heterologous genes, which include at least a) a gene encoding an enzyme from the class of the ribulose-bisphosphate carboxylases (EC number: 4.1.1.39) (RuBisCO gene); and b) a gene encoding an enzyme from the class of the ribulose phosphate kinases (EC number: 2.7.1.19) (PRK gene), which is expressing; wherein the yeast optionally comprises a heterologous expression construct expressing a gene of interest (GOI) and/or wherein each of said RuBisCO gene and said PRK gene, is fused with a nucleotide sequence encoding a peroxisomal targeting signal (PTS).

### TECHNICAL FIELD

The invention relates to yeast incorporating heterologous genes, which expresses a synthetic Calvin cycle, and methods of culturing the yeast while fixing carbon dioxide.

### BACKGROUND OF THE INVENTION

Green-house gas emissions and the connected climate change are among the most pressing problems of our society. Using CO₂ as carbon source for industrial production processes instead of fossil resources could limit green-house gas emissions significantly. Biotechnology is one key-technology for the bio-based economy. Many feed and food applications as well as base chemical- and pharmaceutical productions commence with microorganisms as catalysts. These processes are mostly based on plant derived resources, such as sugars, but they are rarely based on atmospheric CO₂ directly. However, increased use of plant derived carbon is connected to land use change and other detrimental effects on our planet. Direct carbon dioxide fixation of the production organisms is therefore desirable. Most naturally carbon fixing organisms use (sun) light as energy source, which makes them entirely independent from organic carbon for growth, which is beneficial. However, in liquid microbial cultures, light distribution can be a huge technical problem and usually growth and production rates of such organisms are very low. The classical host organisms for biotech productions are much more efficient in terms of production rates, but they rely on organic carbon.

The genetic engineering of carbon dioxide fixation pathways was already shown to be feasible for yeast systems like Saccharomyces cerevisiae or the bacterial system Escherichia coli. In the yeast, S. cerevisiae it was shown that carbon dioxide fixation is feasible together with a simultaneous maltose or xylose fermentation leading to enhanced ethanol production (Guadalupe-Medina et al. Biotechnol. Biofuels 2013, 6:125; Li et al. Scientific Reports 2017, 7:43875). However, in this system it is not possible to decouple the carbon assimilation from the energy supply in form of NADH. Therefore, the biomass is assimilated not only from CO₂ but also from xylose, maltose or other sugars like glucose and galactose.

In E. coli, a functional Calvin cycle yielding biomass was engineered, which was decoupled from an energy supplying pathway yielding ATP and NADH. Here, pyruvate was used as energy yielding substrate. However, first engineered clones needed further evolution steps to enable growth in the presence of CO₂ and pyruvate (Antonovsky et al. Cell 2016, 166:115-125). There is no yeast strain capable of high carbon dioxide assimilation only in the presence of a second single carbon molecule (like methanol). Methanol is a valuable renewable raw material which can be also formed from fixated carbon dioxide by applying green energy.

WO2015/177800A2 discloses recombinant microorganisms, e.g. bacteria or yeast, capable of carbon fixation. The relevant genes, such as RuBisCO, are expressed in the cytosol. Besides carbon dioxide, an organic carbon source, such as a pentose, hexose or an organic acid is necessary for biomass production.

US2017/0002368A1/WO2015/107469A1 disclose yeasts modified to express a functional type I RuBisCO enzyme, and a class II phosphoribulokinase. It is disclosed that the expression of these enzymes recreates a Calvin cycle is said yeast to enable the yeasts to use carbon dioxide. As an example S. cerevisiae is engineered expressing a heterologous RuBisCO gene in the cytosol. Besides carbon dioxide, glucose is used as additional carbon source.

Peng-Fei Xia et al. (ACS Synthetic Biology 2016, 6(2):276-283) describe a synthetic reductive pentose phosphate pathway into a xylose-fermenting S. cerevisiae.

Frey et al. (Journal of the American Chemical Society 2016, 138(32)10072-10075) describe a synthetic mimic of a carboxysoome which is a cyanobacterial carbon-fixing organelle, to encapsulate two enzymes, RuBisCO and carbonic anhydrase (CO).

Pichia pastoris (syn. Komagataella sp.) is a well-established microbial host organism. Numerous strain engineering approaches for P. pastoris improved the productivity for various products and effort was also dedicated to promoters for production purposes. It is well known for its high protein secretion capacity and multiple proteins are currently produced in this microbial cell factory (Gasser et al. Microb. Cell Fact. 2013, 14:196). Recently, it was described how the methylotrophic lifestyle is accomplished in this yeast (Rußmayer et al. BMC Biol. 2015, 13:80).

It would be highly desirable to allow widely used microbial cell factories to fix carbon, and to combine high production rates with a low demand of plant derived carbon. The aim is to provide a chassis cell for bio-based productions, which is characterized by high growth and production rates, but a lower carbon source demand than the currently used strains. Such chassis cells could be used to produce chemicals or pharmaceutical proteins, with a low carbon foot print.

### SUMMARY OF THE INVENTION

It is the object to engineer an improved microorganism which is capable of fixing carbon dioxide, for use in producing biomass and bio-based productions.

The object is solved by the subject of the claims and further described herein.

According to the invention, there is provided a yeast expressing a synthetic Calvin cycle incorporating heterologous genes, for biomass production, or for use as a host cell to produce a series of different product classes including (small) metabolites, chemicals, recombinant proteins or cellular biomass.

According to a specific embodiment, the yeast comprises a nucleotide sequence expression system expressing a synthetic Calvin cycle comprising heterologous genes of the synthetic Calvin cycle, which include at least

a) a gene encoding an enzyme from the class of the ribulose-bisphosphate carboxylases (EC number: 4.1.1.39) (RuBisCO gene); and

b) a gene encoding an enzyme from the class of the ribulose phosphate kinases (EC number: 2.7.1.19) (PRK gene);

optionally wherein each of said RuBisCO gene and said PRK gene, is fused with a nucleotide sequence encoding a peroxisomal targeting signal (PTS),

optionally wherein the yeast further comprises a heterologous expression construct expressing a gene of interest (GOI).

The PTS facilitates expression of the respective genes into the yeast peroxisome. The expression of the RuBisCO and PRK genes in the yeast peroxisomes has advantageously proven to support biomass assimilation only from carbon dioxide. Thus, a carbon fixating yeast strain could be engineered which contains all necessary enzymes to enable growth on carbon dioxide.

Yet, according to a specific embodiment, the yeasts expresses the synthetic Calvin cycle into the cytosol. Such embodiment may employ one or more of, or each of the heterologous genes of the synthetic Calvin cycle without a nucleotide sequence encoding a PTS.

According to another specific embodiment, the yeast comprises a nucleotide sequence expression system expressing a synthetic Calvin cycle comprising heterologous genes of the synthetic Calvin cycle, and further comprising a heterologous expression construct expressing a gene of interest (GOI), wherein the synthetic Calvin cycle comprises at least the following heterologous genes:

a) a gene encoding an enzyme from the class of the ribulose-bisphosphate carboxylases (EC number: 4.1.1.39) (RuBisCO gene); and

b) a gene encoding an enzyme from the class of the ribulose phosphate kinases (EC number: 2.7.1.19) (PRK gene).

Specifically, the GOI encodes a protein of interest (POI), or one or more enzymes which transforms a carbon source into a metabolite.

Specifically, said carbon source is a C1 carbon molecule, preferably CO₂, CO₃²⁻, HCO₃⁻ and/or methanol.

Specifically, the synthetic Calvin cycle is functional including all necessary enzymes to assimilate carbon dioxide into biomass and to use carbon dioxide as carbon source, respectively. Besides the heterologous RuBisCO and PRK genes, one or more further endogenous or heterologous genes may be incorporated and expressed by such yeast in support of the Calvin cycle.

Specifically, the yeast described herein comprises one or more endogenous genes in addition to the heterologous genes to complete the synthetic Calvin cycle.

Specifically, the synthetic Calvin cycle comprises one or more further heterologous genes. Specifically, said one or more heterologous genes are any of:

a) a gene encoding an enzyme from the class of the phosphoglycerate kinases (EC number: 2.7.2.3) (PGK1 gene), and/or

b) a gene encoding an enzyme from the class of the glyceraldehyde-3-phosphate dehydrogenases (EC number 1.2.1.12) (TDH3 gene); and/or

c) a gene encoding an enzyme from the class of the triose-phosphate isomerases (EC number 5.3.1.1) (TPI1 gene); and/or

d) a gene encoding an enzyme from the class of the transketolases (EC number 2.2.1.1) (TKL1 gene),

optionally wherein one or more, or each of said PGK1, TDH3, TPI1, and TKL1 gene(s) is/are fused with a nucleotide sequence encoding a PTS.

Alternatively, one or more of said PGK1, TDH3, TPI1, and TKL1 genes are endogenous or autologous to said yeast and may be co-expressed with the heterologous genes.

Specifically, said heterologous genes include said RuBisCO gene, said PRK gene, said PGK1 gene, said TDH3 gene, said TPI1 gene, and said TKL1 gene.

Specifically, the synthetic Calvin cycle comprises the following heterologous genes: said RuBisCO gene, said PRK gene, said PGK1 gene, said TDH3 gene, said TPI1 gene, and said TKL1 gene.

Specifically,

a) said RuBisCO gene is of bacterial origin, preferably of the genus Thiobacillus; and/or

b) said PRK gene is of plant origin, preferably of the family Amaranthaceae; and/or

c) said PGK1 gene is of yeast origin, preferably of the genus Ogataea; and/or

d) said TDH3 gene is of yeast origin, preferably of the genus Ogataea; and/or

e) said TPI1 gene is of yeast origin, preferably of the genus Ogataea; and/or

f) said TKL1 gene is of yeast origin, preferably of the genus Ogataea.

Specifically,

a) said RuBisCO gene is of Thiobacillus denitrificans origin, preferably comprising the enzyme coding nucleotide sequence shown in FIG. 5, SEQ ID NO:1, in particular the nucleotide sequence identified as SEQ ID NO:37, or a functionally active variant of any of the foregoing with at least 90% sequence identity expressing a ribulose-bisphosphate carboxylase; and/or

b) said PRK gene is of Spinacia oleracea origin, preferably comprising the enzyme coding nucleotide sequence shown in FIG. 5, SEQ ID NO:2, in particular the nucleotide sequence identified as SEQ ID NO:38, or a functionally active variant of any of the foregoing with at least 90% sequence identity expressing a ribulose phosphate kinase; and/or

c) said PGK1 gene is of Ogataea polymorpha origin, preferably comprising the enzyme coding nucleotide sequence shown in FIG. 5, SEQ ID NO:3, in particular the nucleotide sequence identified as SEQ ID NO:39, or a functionally active variant of any of the foregoing with at least 90% sequence identity expressing a phosphoglycerate kinase; and/or

d) said TDH3 gene is of Ogataea polymorpha origin, preferably comprising the enzyme coding nucleotide sequence shown in FIG. 5, SEQ ID NO:4, in particular the nucleotide sequence identified as SEQ ID NO:40, or a functionally active variant of any of the foregoing with at least 90% sequence identity expressing a glyceraldehyde-3-phosphate dehydrogenase; and/or

e) said TPI1 gene is of Ogataea parapolymorpha origin, preferably comprising the enzyme coding nucleotide sequence shown in FIG. 5, SEQ ID NO:5, in particular the nucleotide sequence identified as SEQ ID NO:41, or a functionally active variant of any of the foregoing with at least 90% sequence identity expressing a triose-phosphate isomerase; and/or; and/or

f) said TKL1 gene is of Ogataea parapolymorpha origin, preferably comprising the enzyme coding nucleotide sequence shown in FIG. 5, SEQ ID NO:6, in particular the nucleotide sequence identified as SEQ ID NO:42, or a functionally active variant of any of the foregoing with at least 90% sequence identity expressing a transketolase.

Specifically, the nucleotide sequences encoding the respective enzymes and further including the PTS coding sequence are selected from the group consisting of SEQ ID NO:1 to 6. Such sequences include the PTS coding sequence at the 3′ end. Exemplary PTS coding sequences are “TCCAAGTTG” identified as SEQ ID NO:44, or “TCTAAGTTG” (SEQ ID NO:45).

However, it is well understood that the nucleotide sequences may include alternative PTS coding sequences, as further described herein. The PTS provides for expressing the gene and the gene-encoded enzyme, respectively, into the yeast peroxisome. The synthetic Calvin cycle employing enzyme sequences including the PTS is herein referred to as a “peroxisomal Calvin cycle”.

Specifically, the nucleotide sequences encoding the respective enzymes without the PTS coding sequence are selected from the group consisting of SEQ ID NO:37 to 42. In the absence of the PTS coding sequence, the gene-encoded enzymes are targeted into the yeast cytosol. The synthetic Calvin cycle employing enzyme sequences without any PTS is herein referred to as a “cytosolic Calvin cycle”.

According to a specific embodiment, each of said RuBisCO gene and said PRK gene, is fused with a nucleotide sequence encoding a PTS to express a synthetic Calvin cycle in the yeast peroxisomes.

According to an alternative embodiment, one or both of said RuBisCO gene and said PRK gene lack a nucleotide sequence encoding a PTS, such as to express said gene(s) into the cytosol of said yeast.

Specifically, said PTS comprises an amino acid sequence of 3-9 amino acids.

Specifically, said PTS comprises or consists of an amino acid sequence of 3-5 amino acids selected from the group consisting of serine, lysine, leucine, valine, asparagine, aspartic acid, threonine, alanine, arginine, isoleucine, proline, phenylalanine, and methionine, in any combination, such PTS is herein also referred to as PTS1. Specifically, said PTS1 is an amino acid sequence which is any of SKL, VNL, DKL, TKL, ARL, AKI, PNL, ARF, or PML. Selected PTS1 can be optimized for directing the expression of said heterologous genes to the peroxisome compartment of said yeast.

Specifically, said PTS1 comprises or consists of 3-5 amino acids selected from the group consisting of serine, lysine, and leucine,

Specifically, said PTS1 is preferably fused to the carboxy terminus of one of said heterologous gene expression products.

According to a specific embodiment, said PTS comprises or consists of 5-9 amino acids comprising the sequence identified as SEQ ID NO:12, such PTS is herein also referred to as PTS2:

| | |
|---|---|
| SEQ ID NO: 12: | XX(X)nXX, |

wherein X at position 1 is any of R or K;

wherein X at position 2 is any of L, V, or I;

wherein X at position 3 is one or more (n=1-5) amino acids, wherein each is any amino acid;

wherein X at position 4 is any of H or Q;

wherein X at position 5 is any of L or A.

In other words, the sequence identified as SEQ ID NO:12 is the following: XXXXXXXXX,

wherein X at position 1 is any of R or K;

wherein X at position 2 is any of L, V, or I;

wherein X at position 3 is any amino acid;

wherein X at position 4 is no or any amino acid;

wherein X at position 5 is no or any amino acid;

wherein X at position 6 is no or any amino acid;

wherein X at position 7 is no or any amino acid;

wherein X at position 8 is any of H or Q;

wherein X at position 9 is any of L or A.

An exemplary PTS is selected from the group consisting PTS comprising or consisting of an amino acid sequence identified by any of SEQ ID NOs:13-36:

| | |
|---|---|
| SEQ ID NO: 13: | RLXXXXXHL, |

wherein X at position 3 is any amino acid;

wherein X at position 4 is any amino acid;

wherein X at position 5 is any amino acid;

wherein X at position 6 is any amino acid;

wherein X at position 7 is any amino acid;

| | |
|---|---|
| SEQ ID NO: 14: | RLXXXXXHA, |

wherein X at position 3 is any amino acid;

wherein X at position 4 is any amino acid;

wherein X at position 5 is any amino acid;

wherein X at position 6 is any amino acid;

wherein X at position 7 is any amino acid;

| | |
|---|---|
| SEQ ID NO: 15: | RLXXXXXQL, |

wherein X at position 3 is any amino acid;

wherein X at position 4 is any amino acid;

wherein X at position 5 is any amino acid;

wherein X at position 6 is any amino acid;

wherein X at position 7 is any amino acid;

| | |
|---|---|
| SEQ ID NO: 16: | RLXXXXXQA, |

wherein X at position 3 is any amino acid;

wherein X at position 4 is any amino acid;

wherein X at position 5 is any amino acid;

wherein X at position 6 is any amino acid;

wherein X at position 7 is any amino acid;

| | |
|---|---|
| SEQ ID NO: 17: | RVXXXXXHV, |

wherein X at position 3 is any amino acid;

wherein X at position 4 is any amino acid;

wherein X at position 5 is any amino acid;

wherein X at position 6 is any amino acid;

wherein X at position 7 is any amino acid;

| | |
|---|---|
| SEQ ID NO: 18: | RVXXXXXHA, |

wherein X at position 3 is any amino acid;

wherein X at position 4 is any amino acid;

wherein X at position 5 is any amino acid;

wherein X at position 6 is any amino acid;

wherein X at position 7 is any amino acid;

| | |
|---|---|
| SEQ ID NO: 19: | RVXXXXXQV, |

wherein X at position 3 is any amino acid;

wherein X at position 4 is any amino acid;

wherein X at position 5 is any amino acid;

wherein X at position 6 is any amino acid;

wherein X at position 7 is any amino acid;

| | |
|---|---|
| SEQ ID NO: 20: | RVXXXXXQA, |

wherein X at position 3 is any amino acid;

wherein X at position 4 is any amino acid;

wherein X at position 5 is any amino acid;

wherein X at position 6 is any amino acid;

wherein X at position 7 is any amino acid;

| | |
|---|---|
| SEQ ID NO: 21: | RIXXXXXHI, |

wherein X at position 3 is any amino acid;

wherein X at position 4 is any amino acid;

wherein X at position 5 is any amino acid;

wherein X at position 6 is any amino acid;

wherein X at position 7 is any amino acid;

| | |
|---|---|
| SEQ ID NO: 22: | RIXXXXXHA, |

wherein X at position 3 is any amino acid;

wherein X at position 4 is any amino acid;

wherein X at position 5 is any amino acid;

wherein X at position 6 is any amino acid;

wherein X at position 7 is any amino acid;

SEQ ID NO: 23:
RIXXXXXQI,

wherein X at position 3 is any amino acid;

wherein X at position 4 is any amino acid;

wherein X at position 5 is any amino acid;

wherein X at position 6 is any amino acid;

wherein X at position 7 is any amino acid;

SEQ ID NO: 24:
RIXXXXXQA,

wherein X at position 3 is any amino acid;

wherein X at position 4 is any amino acid;

wherein X at position 5 is any amino acid;

wherein X at position 6 is any amino acid;

wherein X at position 7 is any amino acid;

SEQ ID NO: 25:
KLXXXXXHL,

wherein X at position 3 is any amino acid;

wherein X at position 4 is any amino acid;

wherein X at position 5 is any amino acid;

wherein X at position 6 is any amino acid;

wherein X at position 7 is any amino acid;

SEQ ID NO: 26:
KLXXXXXHA,

wherein X at position 3 is any amino acid;

wherein X at position 4 is any amino acid;

wherein X at position 5 is any amino acid;

wherein X at position 6 is any amino acid;

wherein X at position 7 is any amino acid;

SEQ ID NO: 27:
KLXXXXQL,

wherein X at position 3 is any amino acid;

wherein X at position 4 is any amino acid;

wherein X at position 5 is any amino acid;

wherein X at position 6 is any amino acid;

wherein X at position 7 is any amino acid;

SEQ ID NO: 28:
KLXXXXXQA,

wherein X at position 3 is any amino acid;

wherein X at position 4 is any amino acid;

wherein X at position 5 is any amino acid;

wherein X at position 6 is any amino acid;

wherein X at position 7 is any amino acid;

SEQ ID NO: 29:
KVXXXXXHV,

wherein X at position 3 is any amino acid;

wherein X at position 4 is any amino acid;

wherein X at position 5 is any amino acid;

wherein X at position 6 is any amino acid;

wherein X at position 7 is any amino acid;

SEQ ID NO: 30:
KVXXXXXHA,

wherein X at position 3 is any amino acid;

wherein X at position 4 is any amino acid;

wherein X at position 5 is any amino acid;

wherein X at position 6 is any amino acid;

wherein X at position 7 is any amino acid;

SEQ ID NO: 31:
KVXXXXXQV,

wherein X at position 3 is any amino acid;

wherein X at position 4 is any amino acid;

wherein X at position 5 is any amino acid;

wherein X at position 6 is any amino acid;

wherein X at position 7 is any amino acid;

SEQ ID NO: 32:
KVXXXXXQA,

wherein X at position 3 is any amino acid;

wherein X at position 4 is any amino acid;

wherein X at position 5 is any amino acid;

wherein X at position 6 is any amino acid;

wherein X at position 7 is any amino acid;

SEQ ID NO: 33:
KIXXXXXHI,

wherein X at position 3 is any amino acid;

wherein X at position 4 is any amino acid;

wherein X at position 5 is any amino acid;

wherein X at position 6 is any amino acid;

wherein X at position 7 is any amino acid;

SEQ ID NO: 34:
KIXXXXXHA,

wherein X at position 3 is any amino acid;

wherein X at position 4 is any amino acid;

wherein X at position 5 is any amino acid;

wherein X at position 6 is any amino acid;

wherein X at position 7 is any amino acid;

SEQ ID NO: 35:
KIXXXXXQI,

wherein X at position 3 is any amino acid;

wherein X at position 4 is any amino acid;

wherein X at position 5 is any amino acid;

wherein X at position 6 is any amino acid;

wherein X at position 7 is any amino acid;

SEQ ID NO: 36:
KIXXXXXQA,

wherein X at position 3 is any amino acid;

wherein X at position 4 is any amino acid;

wherein X at position 5 is any amino acid;

wherein X at position 6 is any amino acid;

wherein X at position 7 is any amino acid;

Specifically, said PTS is fused to any of the amino terminus or carboxy terminus of said heterologous gene expression products, or fused such that the nucleotide sequence encoding the PTS is incorporated into the gene sequence at any position, thereby leading to peroxisomal expression.

Specifically, the yeast is further engineered to express helper factors, such as molecular chaperones.

Specifically, the yeast comprises further heterologous genes expressing one or more molecular chaperones in the cytosol of said yeast, which chaperones assist the covalent folding and/or assembly of at least one of said enzymes. Specifically, the chaperones are helper factors for the correct folding of the RuBisCO enzyme, thereby supporting the enzyme function.

Specifically, said chaperones are selected from the group of heat shock proteins and proteins of the chaperonin family, preferably of bacterial origin.

Specifically, said chaperones are at least

a) GroEL of Escherichia coli origin, preferably encoded by the chaperone coding nucleotide sequence shown in FIG. 5, SEQ ID NO:7, in particular the nucleotide sequence identified as SEQ ID NO:43, or a functionally active variant of any of the foregoing with at least 90% sequence identity expressing a molecular chaperone; and

b) GroES, of Escherichia coli origin, preferably encoded by a nucleotide sequence identified as SEQ ID NO:8, or a functionally active variant thereof with at least 90% sequence identity expressing a molecular chaperone.

Specifically, methylotrophic and non-methylotrophic yeasts, e.g. of the genus Pichia, comprise endogenous genes PGK1, TDH3, TPI1, and TKL1 which can be expressed in the peroxisomal compartment of the yeast in addition to the heterologous RuBisCO and PRK genes, and the endogenous genes GroEL and GroES in the yeast cytosol, thereby expressing the functional Calvin cycle. Yet, overexpression of one or more of the endogenous genes may be advantageous. Thus, any of the endogenous genes expressing relevant enzymes of the Calvin cycle may be overexpressed e.g., by suitable promoter engineering or by co-expressing helper factors. Alternatively, a heterologous gene expressing the same type of enzyme as the endogenous one may additionally be introduced into the yeast, or substitute the endogenous one.

In another embodiment, it is advantageous that each of the RuBisCO, PRK, PGK1, TDH3, TPI1, and TKL1 genes is heterologous to the yeast and incorporated into the genome of the yeast for expression in the host cell peroxisome. Further, each of the GroEL and GroES genes is heterologous to the yeast and incorporated into the genome of the yeast for expression in the host cell cytosol.

Specifically, one or more of said heterologous genes of the synthetic Calvin cycle or said chaperones, or of any sequences used in the heterologous expression construct expressing a gene of interest (GOI), in particular the GOI, are codon-optimized for expression in said yeast. Specifically, each of the heterologous genes described herein is codon-optimized.

Specifically, said heterologous genes are operably linked to a promoter. Specifically, each of said heterologous genes is operably linked to a promoter.

Specific promoter-types include at least constitutive, inducible, synthetic, compartment-specific and development-stage specific promoters.

Specifically, said promoter is any of a methanol-inducible promoter, which promotes the expression of natively methanol-induced genes (Gasser, B., Steiger, M. G., & Mattanovich, D. (2015). Methanol regulated yeast promoters: production vehicles and toolbox for synthetic biology. Microbial Cell Factories, 14:196).

Specifically, said promoter is any promoter of constitutive type.

According to a specific embodiment, said yeast comprises a further nucleotide sequence expression system expressing a protein of interest (POI), or one or more enzymes transforming a carbon source into a metabolite, specifically an organic small molecule fermentation product, which is produced by a metabolic pathway expressed by the yeast host cell. Specifically, a promoter is operably linked to the GOI, in particular which GOI is a nucleotide sequence encoding the POI or an enzyme used for metabolite production, which promoter is not natively associated with the nucleotide sequence encoding the POI. The POI is specifically a heterologous polypeptide or protein. Specifically, the POI is a eukaryotic protein, preferably a mammalian protein. In specific cases, a POI is a multimeric protein, specifically a dimer or tetramer.

Specifically, the GOI expression cassette further comprises a nucleotide sequence encoding a signal peptide enabling the secretion of a POI, preferably wherein nucleotide sequence encoding the signal peptide is located adjacent to the 5′-end of the nucleotide sequence encoding the POI.

Specifically, said carbon source is a C1 carbon molecule, preferably CO₂, CO₃²⁻⁻, HCO₃⁻ and/or methanol.

Specifically, said metabolite is selected from the group consisting of organic acids, preferably any of citric acid, lactic acid, gluconic acid, formic acid, succinic acid, oxalic acid, malic acid, acetic acid, propionic acid, butyric acid, isobutyric acid, tartaric acid, itaconic acid, ascorbic acid, or fumaric acid; lipids, preferably any of fatty acids, glycerolipids, glycerophospholipids, sphingolipids, sterol, or lipids; alcohols, preferably any of ethanol, butanol, propanol, butanediol, or propanediol; polyols, preferably any of arabitol, erythritol, or xylitol; and carbohydrates, preferably any of glucose, fructose, or xylose.

Specifically, said metabolite is a yeast metabolite produced by a pathway which is naturally-occurring in the yeast, or artificial because employing one or more heterologous gene(s).

Specifically, said POI is selected from the group consisting of therapeutic proteins or industrially relevant technical enzymes. Specifically, said POI from the group of therapeutic proteins is preferably any of antibody molecules or antigen-binding fragments thereof, enzymes and peptides, protein antibiotics, toxin fusion proteins, carbohydrate-protein conjugates, structural proteins, regulatory proteins, vaccines and vaccine-like proteins or particles, process enzymes, growth factors, hormones and cytokines. Specifically, said POI from the group of technical enzymes is preferably any derived from the group of hydrolytic enzymes, transferases, oxidoreductases, lyases, isomerases, or ligases.

A specific POI from the group of hydrolytic enzymes is an enzyme which catalyzes the hydrolysis of a chemical bond, or an engineered variant thereof. Among specific POIs from the group of hydrolytic enzymes are amylases, lipases, mannanases, β-xylanases, pectinases, α-fucosidases, sialidases, phytases, cellulases, or proteases.

A specific POI from the group of transferases is an enzyme which catalyzes the transfer of a functional chemical group, or an engineered variant thereof. Among specific POIs from the group of transferases are methyltransferases, hydroxymethyltransferases, formyltransferases, carboxytransferases, carbamoyltransferases, or transglutaminase.

A specific POI from the group of oxidoreductases is an enzyme which catalyze reductions or oxidations, or an engineered variant thereof. Among specific POIs from the group of oxidoreductases are lactate dehydrogenases, glucoseoxidases, laccases, peroxidases, or polyphenol oxidases.

A specific POI from the group of lyases is an enzyme which chemical bonds in the form of C—O, C—C or C—N, or an engineered variant thereof. Among specific POIs from the group of lyases are pyruvate decarboxylase, or aspartate ammonia lyase.

A specific POI from the group of isomerases is an enzyme which converts one chemical isoform to another, or an engineered variant thereof. Among specific POIs from the group of isomerases are protein disulfide isomerases, or xylose isomerases. A specific POI from the group of ligases is an enzyme which catalyzes the formation of covalent bonds, or an engineered variant thereof. Among specific POIs from the group of ligases are sucrose synthase, or gamma-glutamylcysteine synthetase.

A specific POI is an antigen-binding molecule such as an antibody, or a fragment thereof. Among specific POIs are antibodies such as monoclonal antibodies (mAbs), immunoglobulin (Ig) or immunoglobulin class G (IgG), heavy-chain antibodies (HcAb's), or fragments thereof such as fragment-antigen binding (Fab), Fd, single-chain variable fragment (scFv), or engineered variants thereof such as for example Fv dimers (diabodies), Fv trimers (triabodies), Fv tetramers, or minibodies and single-domain antibodies like VH or VHH or V-NAR. Further antigen-binding molecules may be selected from (alternative) scaffold proteins such as e.g. engineered Kunitz domains, Adnectins, Affibodies, Anticalins, and DARPins.

Specifically, said yeast is a recombinant cell or cell line, also referred to as host cell or host cell line. Specifically, said yeast is a production cell line, producing a POI or metabolite. Specifically, the yeast expressing a POI or metabolite is provided as a chassis cell, ready for preparing a production cell line by introducing relevant gene(s) encoding the POI or metabolic pathway into the yeast genome or by episomal expression.

Specifically, said yeast, herein also referred to a host cell, is a methylotrophic yeast, derived from a methylotrophic yeast, or engineered from a wild-type methylotrophic yeast.

The capacity to grow on methanol as a single carbon sources turned out to be advantageous to engineer the Calvin-cycle into this organism, because most of the relevant enzymes except for RuBisCO and PRK and four accessory steps are already present in the peroxisome of methylotrophic yeast.

Specifically, said yeast is of the genus selected from the group consisting of Pichia, Komagataella, Hansenula, Ogataea, Candida, and Torulopsis.

Specifically, said yeast is selected from the group consisting of Pichia pastoris Komagataella pastoris, K. phaffii, and K. pseudopastoris. A specifically preferred yeast is Pichia pastoris, Komagataella pastoris, K. phaffii, or K. pseudopastoris, such as e.g., any of the P. pastoris strains CBS 704 (Centraalbureau voor Schimmelcultures, NL), CBS 2612, CBS 7435, CBS 9173-9189, DSMZ 70877, X-33, GS115, KM71 and SMD1168.

Specifically, said yeast is produced by engineering the endogenous DAS1 locus and/or DAS2 locus to knock out the respective endogenous gene function or expression.

Specifically, said yeast is produced by engineering the endogenous AOX1 locus to knock out the respective endogenous gene function or expression, e.g. in addition to engineering the endogenous DAS1 locus and/or DAS2 locus.

Upon producing a knock-out of one or more of said endogenous genes in a wild-type methylotrophic yeast, the yeast no more comprises the genes encoding the first steps of assimilation in the methanol-utilizing pathway, but is still designated “methylotrophic” for the purpose described herein.

Specifically, the assimilative branch of the methanol-utilizing pathway is knocked out by introducing one or more of the heterologous genes described herein into any one of or both, the DAS1 and DAS2 loci, and optionally also into the AOX1 locus. According to a specific example, both genes, the RuBisCO and PRK genes, are incorporated into only one of the AOX1 and/or the DAS1 and/or the DAS2 locus.

In another embodiment, one or more of the heterologous genes described herein are introduced (e.g. by a suitable knock-in method) without interfering or interrupting any endogenous genes of the methanol utilizing pathway.

Specifically, at least two native genes of Pichia pastoris, particularly DAS1 (ORF ID: PP7435_Chr3-0352) and DAS2 (ORF ID: PP7435_Chr3-0350) are replaced by said heterologous genes.

Specifically, the native gene of P. pastoris AOX1 (ORF ID: PP7435_Chr4-0130) is replaced by any of said heterologous genes.

Specifically, three genes in the P. pastoris genome are deleted, namely AOX1, DAS1 and DAS2, and the following heterologous genes are integrated PGK1, TDH3, TPI1, PRK, TKL, GroEL, GroES and RuBisCO into the genome, in particular at the AOX1, DAS1 and DAS2 knock-out sites.

Specifically, TDH3, PRK and PGK1 are integrated in the AOX1 locus under control of the P_{AOX1}, P_{FDH1} and P_{ALD4} promoter. Specifically, RuBisCO, GroEL, and GroES are introduced into the DAS1 locus under control of the P_{DAS1}, P_{PDC1} and P_{RPP1b} promoter. Specifically, TKL1 and TPI1 are introduced into the DAS2 locus under control of the P_{DAS2} and P_{SHB17} promoter.

Specifically, promoters are chosen for expressing the heterologous genes, which are endogenous to the cell at the respective locus of gene integration. Specifically, a native endogenous promoter is used to express one or more of the heterologous genes, e.g. native P_{AOX1} and/or P_{DAS1}, and/or P_{DAS2} of P. pastoris.

Alternatively, exogenous or synthetic promoters can be used.

Specifically, allogenic promoters (of the same species, but introduced at a different location) may be used. Alternatively, promoters can be used which are heterologous to the yeast host cell. Exemplary allogenic promoters are any of promoters of endogenous genes, which are preferably induced by methanol (e.g. promoter sequence of SHB17 (ORF ID: PP7435_Chr2-0185), P_{SHB17} is 500-1000 bps upstream of the coding sequence) (Gasser, B., Steiger, M. G., & Mattanovich, D. (2015). Methanol regulated yeast promoters: production vehicles and toolbox for synthetic biology. Microbial Cell Factories, 14:196).

According to a specific embodiment, a promoter controlling expression of one or more of said heterologous genes is methanol-inducible. Exemplary promoters are any of P_{SHB17:} (PP7435_chr2 (340617 . . . 341606), P_{ALD4:} PP7435_chr2 (1466285 . . . 1467148), P_{FDH1:} PP7435_chr3 (423504 . . . 424503), P_{AOX1:} PP7435_chr4 (237941 . . . 238898), P_{DAS1:} PP7435_chr3 (634140 . . . 634688), P_{DAS2:} PP7435_chr3 (632201 . . . 633100), P_{PMP20} PP7435_Chr1-1351 (2418089 . . . 2419089), P_{FBA1-2} PP7435_Chr1 (1163622 . . . 114622), P_{PMP47} PP7435_Chr3 (2033195 . . . 2034195), P_{FLD} PP7435_Chr3 (262519 . . . 263519), P_{FGH1} PP7435_Chr3 (555586 . . . 556586), P_{TAL1-2} cbs7435 (644081 . . . 645081), or any other promoter sequence of a methanol-induced gene (Gasser, B., Steiger, M. G., & Mattanovich, D. (2015). Methanol regulated yeast promoters: production vehicles and toolbox for synthetic biology. Microbial Cell Factories, 14:196).

According to another specific embodiment, a promoter controlling expression of one or more of said heterologous genes is constitutive. Exemplary promoters are any of P_{GAP} PP7435_Chr2 (1585003 . . . 1586003), P_{TEF2} PP7435_Chr1 (2751497 . . . 2752497), P_{RPL2A} PP7435_Chr4 (1576422 . . . 1577422), P_{CS1} PP7435_Chr1 (4023 . . . 5023), P_{FBA1-1} PP7435_Chr1 (679746 . . . 680746), P_{RPP1B} PP7435_Chr4 (46235 . . . 463235), P_{GPM1} PP7435_Chr3 (646226 . . . 647226), P_{PDC1} PP7435_Chr3 (1860826 . . . 1861826), P_{POR1} PP7435_Chr2 (737738 . . . 738738), P_{LAT1} PP7435_Chr1 (637999 . . . 638999), P_{PpPfk} PP7435_Chr4 (1169499 . . . 1170499) or P_{ADH2} PP7435_Chr2 (1519404 . . . 1520404).

Specifically, the yeast is engineered such that each of the heterologous genes described herein is under the control of a promoter that is not natively associated with said heterologous gene.

The invention further provides for a method of culturing the yeast described herein in a cell culture, comprising culturing the yeast in the growing phase using gaseous carbon dioxide and/or dissolved CO₃²⁻ and/or HCO₃⁻ compounds as a carbon source, thereby obtaining accumulated yeast biomass in the cell culture.

Specifically, the yeast biomass is accumulated to at least 0.1 g/L cell dry weight, more preferably at least 1 g/L cell dry weight, preferably at least 10 g/L cell dry weight. Typically, accumulated yeast biomass is cultured in a fermentation device, wherein the yeast is cultured between 10 to 20 g/L cell dry weight.

According to a specific embodiment, the recombinant yeast is cultured under batch, fed-batch or continuous culturing conditions, and/or in media containing gaseous carbon dioxide and/or dissolved CO₃²⁻ and/or HCO₃⁻ compounds, e.g. as a sole carbon source, or in combination with one or more supplemental carbon source(s).

Specifically, a batch phase is performed as a first step a), and the fed-batch phase or a continuous phase is performed as a second step b).

Specifically, the second step b) employs a feed medium in a fed-batch or continuous phase that provides a supplemental carbon source, preferably a C1 carbon source.

According to a specific aspect, the yeast is cultured in a fed-batch mode.

Specifically, the yeast incorporates said heterologous genes operably linked to a promoter, preferably wherein the promoter is inducible by methanol, and wherein said growing phase follows upon adding methanol to the culture medium, thereby inducing the expression of a functional Calvin cycle.

Specifically, expression of the functional Calvin cycle is into the peroxisome, if the respective heterologous enzyme coding nucleotide sequences are fused to PTS sequences.

Alternatively, expression of the functional Calvin cycle is into the cytosol, if the respective heterologous enzyme coding nucleotide sequences are not fused to PTS sequences.

Specifically, the method further comprises culturing said accumulated yeast biomass in a production phase using a carbon source to produce said POI and metabolite, respectively, e.g. as a sole carbon source or in combination with one or more supplemental carbon source(s).

According to a specific aspect, the invention provides for a method of producing a POI utilizing such yeast transformed with a heterologous gene of interest encoding the POI, wherein the yeast is expressing a synthetic Calvin cycle further described herein.

According to another specific aspect, the invention provides for a method of producing a yeast metabolite utilizing such yeast transformed with a heterologous gene of interest encoding an enzyme used by the yeast for metabolite production, wherein the yeast is expressing a synthetic Calvin cycle further described herein.

According to another specific aspect, the invention provides for a method of producing yeast biomass utilizing such yeast expressing a synthetic Calvin cycle further described herein.

Specifically, said growing phase is performed in a batch mode and said production phase is performed in a feeding batch or continuous mode.

As described herein, yeast was undergoing metabolic engineering to introduce a synthetic (or fully or partly heterologous) carbon fixation module. Expression of heterologous genes for the creation of the Calvin cycle is directed to the peroxisomes, which turned out to be highly effective. Thereby carbon dioxide could be used as a sole carbon source for biomass production. Specifically, the culture medium has been gassed with carbon dioxide.

According to a specific aspect, the invention further provides for a method of producing an organic product, such as a POI or a metabolite, in a yeast which comprises a synthetic Calvin cycle described herein, wherein at least 20% or at least any of 30%, 40%, 50%, 60%, 70%, 80%, 90% or 100% of the product's total organic carbon is from a carbon source which is gaseous carbon dioxide and/or dissolved CO₃²⁻ and/or HCO₃⁻ compounds. Specifically, such carbon source is used as structural carbon, i.e. carbon atoms built into the structure of the organic substance.

According to a specific aspect, the invention further provides for the use of a yeast described herein for producing a POI and/or metabolite using a carbon source which is gaseous carbon dioxide and/or dissolved CO₃²⁻ and/or HCO₃ compounds e.g., as a sole carbon source or in combination with a supplemental carbon source.

It surprisingly turned out that a genetically engineered strain of the yeast Pichia pastoris could be provided which can accumulate biomass, and fix atmospheric carbon dioxide, while the energy is provided by organic carbon. All reactions of a functional Calvin cycle could advantageously be either targeted to the peroxisome or the cytosol, so that the entire C1 assimilation pathway could be localized in the same cellular compartment, and separated from the common carbon metabolism. Thereby, the carbon metabolism was split into two subsystems: one depending on CO₂ for biomass assimilation and the other dependent on a carbon source, such as methanol, as an energy source, e.g. for the generation of reducing equivalents. This modular design enabled replacement of the energy supplying module by another. For instance, other reduced substrates like hydrogen could be used to generate NADH thus allowing a net carbon fixation.

According to a specific example, a novel P. pastoris strain was created by metabolic engineering, which has the ability to efficiently assimilate carbon dioxide into biomass. With this technology, it was possible to utilize carbon dioxide as valuable resource for biotechnological applications and to assimilate it into different bio-based products. According to the example, the engineered P. pastoris strain has the ability to use CO₂ as sole carbon source. For energy supply, any source yielding NADH can be used due to a modular metabolic design. Methanol oxidation can be used for this purpose. This yeast system significantly outcompetes other engineered systems for CO₂-fixation like Escherichia coli or Saccharomyces cerevisiae.

The advantage of a yeast or P. pastoris platform utilizing the synthetic Calvin cycle described herein, is the ability to accumulate biomass to very high cell densities exceeding 100 g/L. Thus, high space-time yields are in reach for a CO₂-fixation platform based upon this microbial chassis. Furthermore, conventional bioreactors can be used for the cultivation without the need for specialized photobioreactors. This platform can be developed for different product classes including small metabolites, chemicals, recombinant proteins or cellular biomass.

In an example described herein, the carbon dioxide assimilation pathway was targeted into the peroxisome, thereby replacing the natural formaldehyde assimilation capacity of P. pastoris. Methanol was only used to generate reduction equivalents in the form of NADH. This energy generating step was performed for the net fixation of carbon dioxide. However, alternative reduced substrates can be used, which can yield NADH (e.g. glycerol, glucose, xylose, maltose, xylitol, arabitol, sorbitol, ethanol).

In another example, the carbon dioxide assimilation pathway was targeted into the cytosol. Such yeast was advantageously used for the production of a POI or yeast metabolite using an artificial expression system.

As an example, the coding sequences of genes listed in the Table 5 (example 2) were integrated into Pichia pastoris. C-terminal protein sequences of the heterologous genes RuBisCO, PRK, PGK1, TDH3, TPI1 and TKL1, respectively, were engineered to contain a PTS, which directed the expression of said genes in the peroxisomes. GroEL and GroES genes encoding helper factors (chaperones) were expressed in the cytosol.

As further described in the Examples section, three genes in the Pichia genome were deleted namely aox1, das1 and das2, and eight genes were integrated into the genome. In brief, the heterologous genes, each derived from species other than P. pastoris, which are PGK1, TDH3, TPI1, PRK, TKL, GroEL, GroES, and RuBisCO, were integrated into the genome at the three deletion sites of AOX1, DAS1 and DAS2. All introduced genes which are part of the Calvin cycle (in particular the PGK1, TDH3, TPI1, PRK, TKL, and RuBisCO genes) have been engineered to contain a C-terminal peroxisome targeting signal (PTS) to enable the compartmentalization to the peroxisome. GroEL, GroES did not contain the PTS and were expressed in the cytosol. The coding sequences of the heterologous genes were combined with suitable promoter and terminator sequences, such as methanol inducible promoters from P. pastoris and terminator sequences from P. pastoris. All expression cassettes were flanked with the respective integration sites to replace the three aforementioned genes, aox1, das1 and das2.

According to further Examples, three genes in the Pichia genome were deleted namely aox1, das1 and das2 and eight genes were integrated into the genome. In brief, PGK1, TDH3 TPI1, PRK, TKL GroEL, GroES and RuBisCO were integrated into the genome at the three deletion sites of AOX1, DAS1 and DAS2. The coding sequences (CDS) of the genes were combined with methanol inducible promoters from P. pastoris and terminator sequences from P. pastoris. All expression cassettes (promoter, CDS, terminator) were constructed by Golden Gate cloning and flanked with the respective integration sites to replace the three aforementioned genes, aox1, das1 and das2. To facilitate integration by homologous recombination at the three mentioned loci, a CRISPR/Cas9 strategy was followed. In brief, a plasmid carrying an expression cassette for Cas9 and a gRNA expression construct was co-transformed alongside the linear DNA integration fragment. The gRNA was designed to target either the aox1, das1 or das2 locus close to the 5′ end of the coding sequence (aox1, das1) or to the 5′ end (das2). After screening for the strain with the integrated DNA construct by colony PCR, the CRIPSR/Cas9 plasmid is readily lost by releasing the selection pressure. Thus a strain was created carrying only the integrated expression cassette, without the need for any additional selection marker. The correct integration was verified by PCR and Sanger sequencing of the three integration loci.

It could be shown that the carbon assimilation can also take place with a metabolic pathway localized to the cytosol.

By following a metabolic engineering strategy (deletion of three genes and expression of eight proteins in the cytosol of P. pastoris), it was possible to establish a functional Calvin cycle in the yeast Pichia pastoris. This enabled the fixation of carbon dioxide and its assimilation into the biomass of Pichia pastoris. Methanol was only used to generate reduction equivalents in the form of NADH, as the assimilation pathway of methanol was blocked due to a DAS1, DAS2 deletion. This energy generating step was necessary for the net fixation of carbon dioxide. However, also other reduced substrates can be used as an alternative, which can yield NADH (e.g. H₂).

### FIGURES

FIG. 1: Engineered GaT_pp_10 strains (GaT_pp_10a and GaT_pp_10b) are able to grow in presence of methanol and CO₂ while GaT_pp_12 and GaT_pp_13 are not. CBS7435 wt cells grow well in presence of both substrates, since methanol can be utilized for biomass and energy generation. Cells were cultivated in batch phase (16.0 g*L⁻¹) until a cell dry weight (CDW) of ˜10 g*L⁻¹ and then fed with 0.5-1.0% methanol pulses and a constant inflow of 5% CO₂. CDW values are calculated from OD measurements (correlation: 1 OD unit=0.191 g CDW*L⁻¹) and standard error bars indicate the standard error of 4 measurements.

FIG. 2: Growth during methanol uptake rate determination. Only CBS7435 wt and RuBisCO positive GaT_pp_10 strains (GaT_pp_10a and GaT_pp_10b) were able to grow on methanol and CO₂. GaT_pp_12 and GaT_pp_13 strains did not show any growth within the observed timeframe.

FIG. 3: Methanol consumption during uptake rate determination study. On day 6 of cultivation of the fermentation 1 shown in example 4, methanol uptake rates were determined and showed the highest methanol utilization by CBS7435 wt cells followed by the engineered GaT_pp_10 strains (GaT_pp_10a and GaT_pp_10b). Strains lacking RuBisCO (GaT_pp_12 and GaT_pp_13 showed slow methanol utilization (compare corresponding lines in FIG. 2).

FIG. 4: Growth in engineered GaT_pp_10 strain (technical replicates GaT_pp_10a and GaT_pp_10b) depends on the supply of CO₂ as a carbon source. The course of biomass formation in engineered GaT_pp_10 (GaT_pp_10a (circle) and GaT_pp_10b (peak)) is shown compared to the control strain, which lacks RuBisCO (GaT_pp_12a (rectangular) (GaT_pp_12 b (triangle). Cells were cultivated in batch phase (16.0 g glycerol*L⁻¹, starting at t₀) until a CDW of ˜10 g*L⁻¹ and then induced with 0.5% methanol (w/v) (at t₁) and afterwards fed with pulses of 1% (w/v) methanol (t₂ until end of fermentation 2). After induction, only GaT_pp_10b and GaT_pp_12 b were co-fed with 5% CO₂. After 3 days (t₃) and occurrence of pronounced growth (GaT_pp_10b), the CO₂ supply was set to 0% for GaT_pp_10b and GaT_pp_12 b and increased to 5% for GaT_pp_10a and GaT_pp_12a. CDW values are calculated from OD measurements (correlation: 1 OD unit=0.191 g CDW*L⁻¹) and standard error bars indicate the standard error of 4 measurements.

FIG. 5: Nucleotide sequences of the heterologous genes

PTS: underlined

Stop codon: TAA in bold and italic

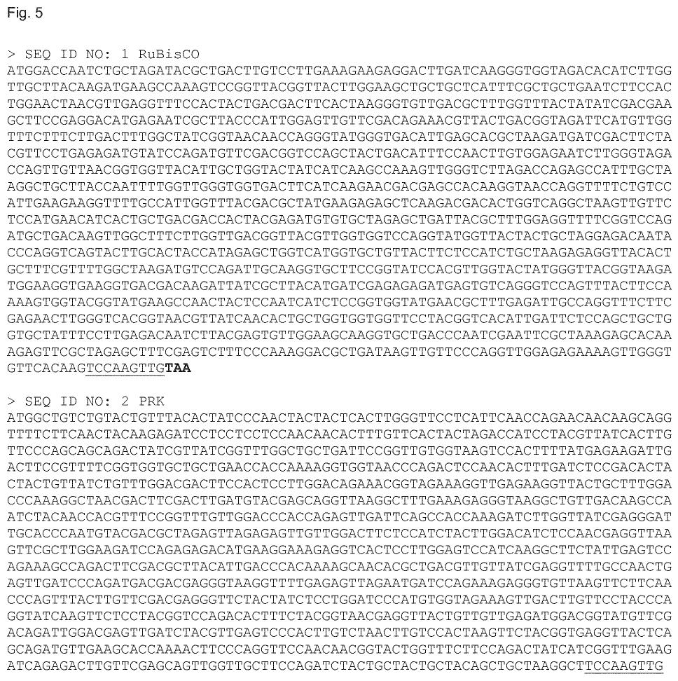

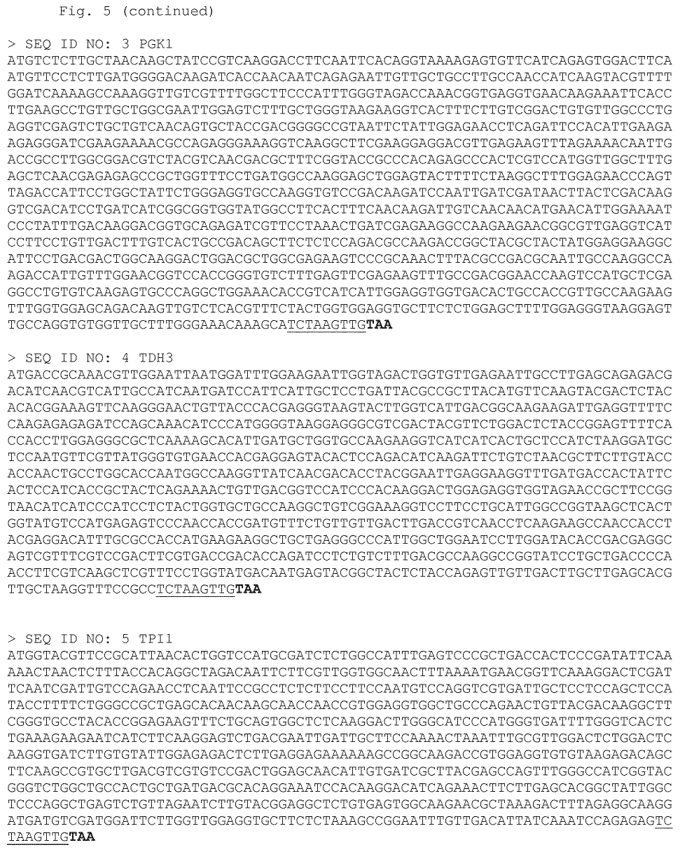

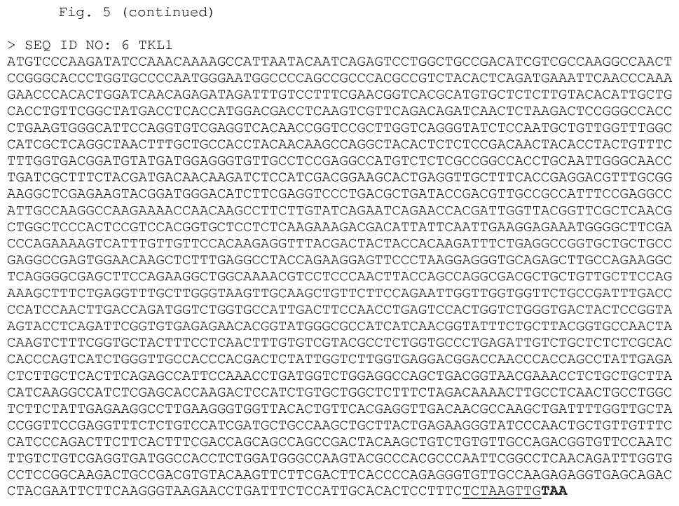

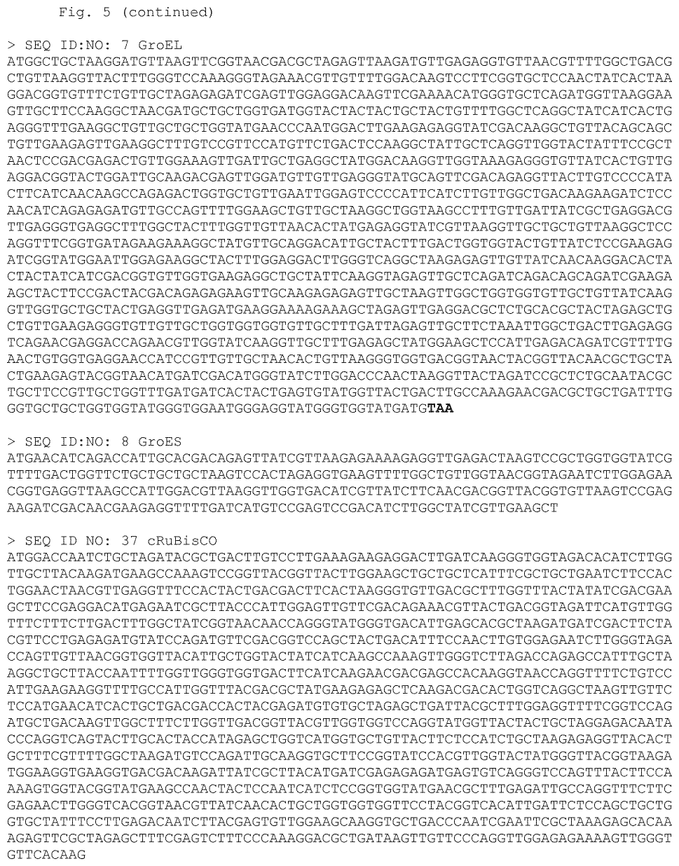

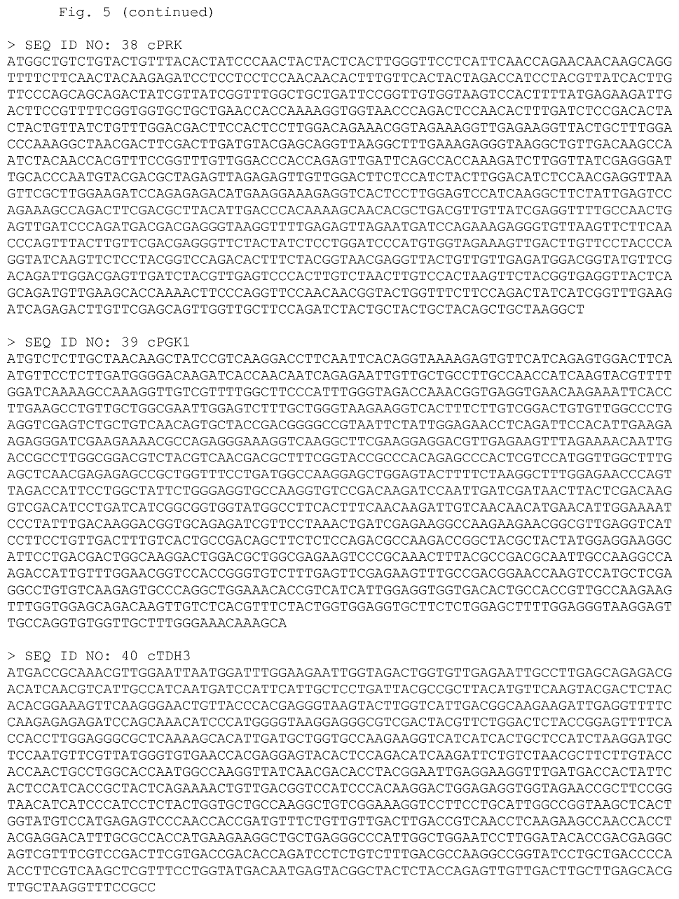

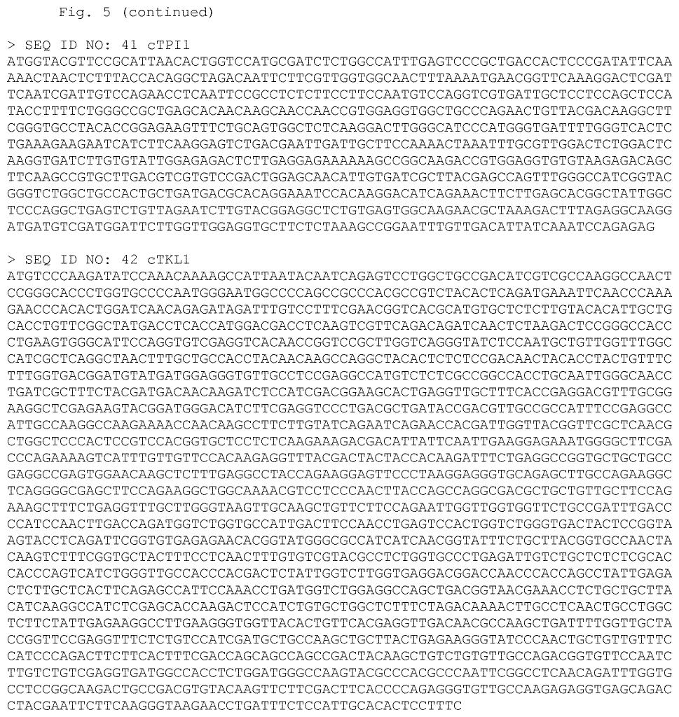

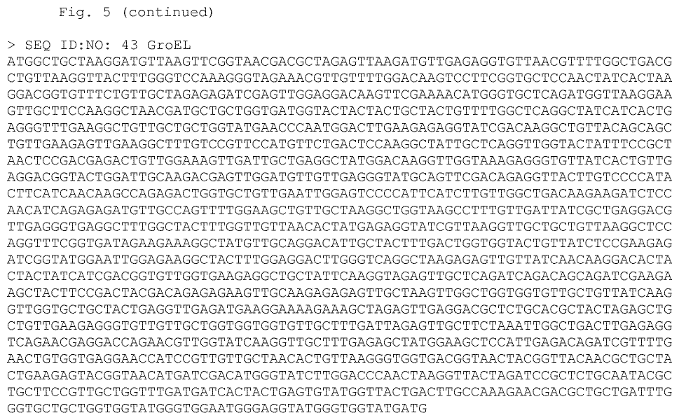

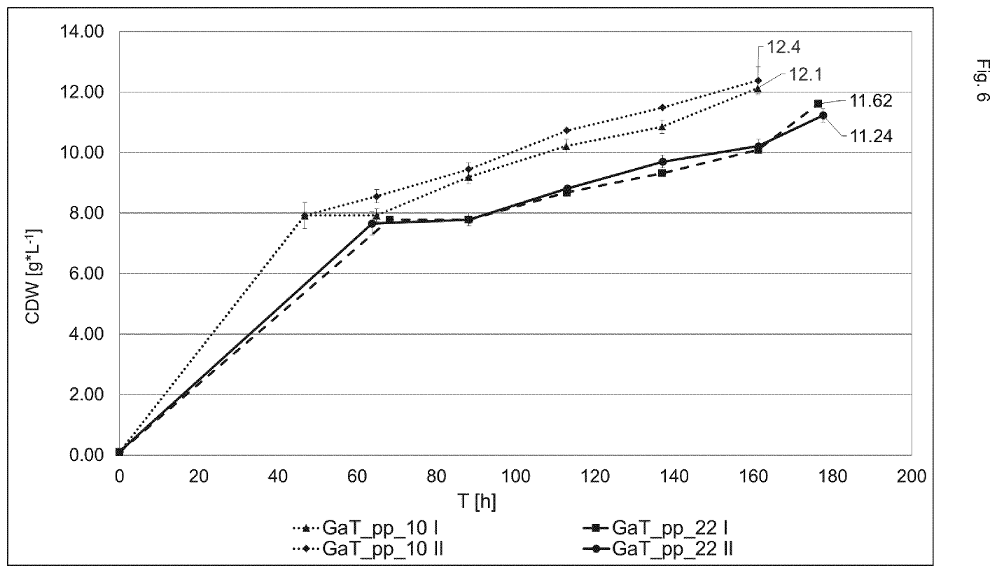

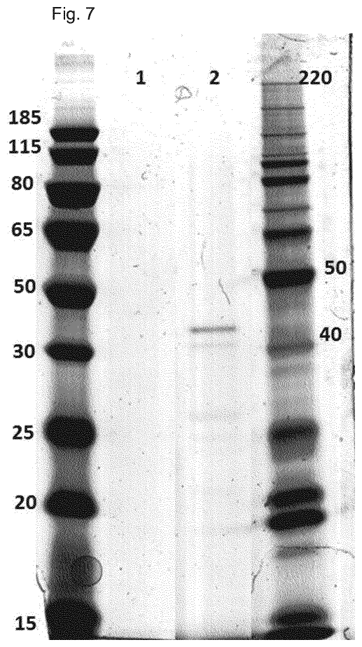

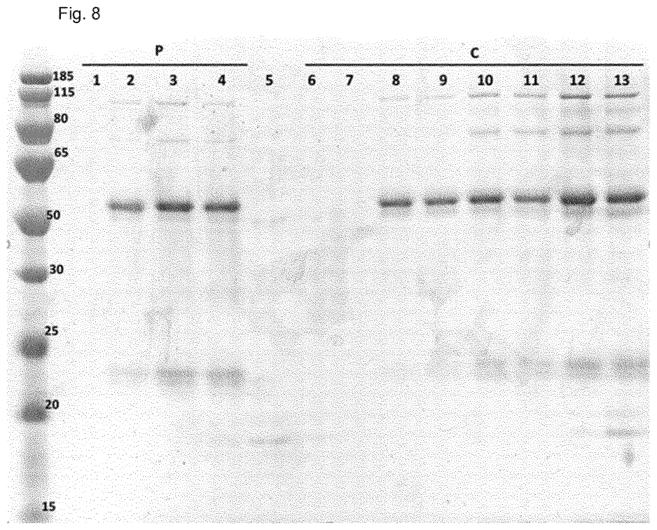

As indicated in FIG. 5, some of the gene encoding sequences additionally comprise a PTS coding nucleotide sequence and/or a stop codon. It is well understood that the gene encoding sequences may be used with or without such PTS coding sequence, and optionally with the TAA or alternative stop codon, if any.

SEQ ID NO:1: nucleotide sequence of the RuBisCO enzyme Form II of Thiobacillus denitrificans. The nucleotide sequence identified as SEQ ID NO:1 consists of the enzyme coding sequence starting at the 5′ end, followed by the PTS coding sequence “TCCAAGTTG” (SEQ ID NO:44), and the stop codon “TAA” at the 3′ end).

SEQ ID NO:2: nucleotide sequence of the PRK enzyme Form II of Spinacia oleracea. The nucleotide sequence identified as SEQ ID NO:2 consists of the enzyme coding sequence starting at the 5′ end, followed by the PTS coding sequence “TCCAAGTTG” (SEQ ID NO:44).

SEQ ID NO:3: nucleotide sequence of the PGK1 enzyme of Ogataea polymorpha. The nucleotide sequence identified as SEQ ID NO:3 consists of the enzyme coding sequence starting at the 5′ end, followed by the PTS coding sequence “TCTAAGTTG” (SEQ ID NO:45), and the stop codon “TAA” at the 3′ end).

SEQ ID NO:4: nucleotide sequence of the TDH3 enzyme of Ogataea polymorpha. The nucleotide sequence identified as SEQ ID NO:4 consists of the enzyme coding sequence starting at the 5′ end, followed by the PTS coding sequence “TCTAAGTTG” (SEQ ID NO:45), and the stop codon “TAA” at the 3′ end).

SEQ ID NO:5: nucleotide sequence of the TPI1 enzyme of Ogataea parapolymorpha. The nucleotide sequence identified as SEQ ID NO:5 consists of the enzyme coding sequence starting at the 5′ end, followed by the PTS coding sequence “TCTAAGTTG” (SEQ ID NO:45), and the stop codon “TAA” at the 3′ end).

SEQ ID NO:6: nucleotide sequence of the TKL1 enzyme of Ogataea parapolymorpha. The nucleotide sequence identified as SEQ ID NO:6 consists of the enzyme coding sequence starting at the 5′ end, followed by the PTS coding sequence “TCTAAGTTG” (SEQ ID NO:45), and the stop codon “TAA” at the 3′ end).

SEQ ID NO:7: nucleotide sequence of the GroEL chaperone protein of Escherichia coli. The nucleotide sequence identified as SEQ ID NO:7 consists of the enzyme coding sequence starting at the 5′ end, followed by the stop codon “TAA” at the 3′ end).

SEQ ID NO:8: nucleotide sequence of the GroES chaperone protein of Escherichia coli. The nucleotide sequence identified as SEQ ID NO:8 consists of the enzyme coding sequence.

SEQ ID NO:37: nucleotide sequence of the RuBisCO enzyme Form II of Thiobacillus denitrificans. The nucleotide sequence identified as SEQ ID NO:37 consists of the enzyme coding sequence without a stop codon.

SEQ ID NO:38: nucleotide sequence of the PRK enzyme Form II of Spinacia oleracea. The nucleotide sequence identified as SEQ ID NO:38 consists of the enzyme coding sequence without a stop codon.

SEQ ID NO:39: nucleotide sequence of the PGK1 enzyme of Ogataea polymorpha. The nucleotide sequence identified as SEQ ID NO:39 consists of the enzyme coding sequence without a stop codon.

SEQ ID NO:40: nucleotide sequence of the TDH3 enzyme of Ogataea polymorpha. The nucleotide sequence identified as SEQ ID NO:40 consists of the enzyme coding sequence without a stop codon.

SEQ ID NO:41: nucleotide sequence of the TPI1 enzyme of Ogataea parapolymorpha. The nucleotide sequence identified as SEQ ID NO:41 consists of the enzyme coding sequence without a stop codon.

SEQ ID NO:42: nucleotide sequence of the TKL1 enzyme of Ogataea parapolymorpha. The nucleotide sequence identified as SEQ ID NO:42 consists of the enzyme coding sequence without a stop codon.

SEQ ID NO:43: nucleotide sequence of the GroEL chaperone protein of Escherichia coli. The nucleotide sequence identified as SEQ ID NO:43 consists of the chaperone coding sequence without a stop codon.

FIG. 6: Engineered GaT_pp_22 strains (GaT_pp_22 I and GaT_pp_22 II) are able to grow in presence of methanol and CO₂. Cells were cultivated in batch phase (15.0 g*L−1) until a cell dry weight (CDW) of ˜8 g*L−1 and then fed with 0.5-1.0% (v/v) methanol pulses and a constant inflow of 5% CO₂. CDW values are calculated from OD measurements (correlation: 1 OD unit=0.191 g CDW*L−1) and standard error bars indicate the standard error of 4 measurements.

FIG. 7: Supernatants of strains expressing Carboxypeptidase B (CpB) (GaT_pp_31). Samples were separated on a NuPAGE™ 10% Bis-Tris Protein Gel (ThermoFischer Scientific, US) in MOPS running buffer and silver stained; 1—Supernatant sample at inoculation of the strain GaT_pp_31 in 0.5% (v/v) methanol containing YNB medium, 2—Supernatant sample after 72 hours after inoculation of the strain GaT_pp_31 (methanol concentration maintained at 1% (v/v)), protein ladder left: PageRuler™ Prestained Protein Ladder (ThermoFischer Scientific, US), protein ladder right: BenchMark™ Protein Ladder (ThermoFischer Scientific, US), picture was post-processed and unnecessary lanes were excised using ImageJ

FIG. 8: Supernatants of strains expressing Human Serum Albumin (HSA) GaT_pp_35 (P) and GaT_pp_38 (C). Samples were separated on a NuPAGE™ 10% Bis-Tris Protein Gel (ThermoFischer Scientific, US) in MOPS running buffer and silver stained

1-4: Supernatant samples of GaT_pp_35 with peroxisomal (P) version of the pathway after 0 hours (1), 24 hours (2), 48 hours (3) and 72 hours (4) of inoculation in YNB supplemented with 0.5% methanol 5: Empty lane, 6-13: Supernatant samples of GaT_pp_38 with cytosolic (P) version of the pathway after 0 hours (6,7), 24 hours (8,9), 48 hours (10,11) and 72 hours (12,13) of inoculation for two different clones of GaT_pp_38 (Clone 1: 6/8/10/12, Clone 2: 7/9/11/13), protein ladder left: PageRuler™ Prestained Protein (ThermoFischer Scientific, US)

### DETAILED DESCRIPTION OF THE INVENTION

Specific terms as used throughout the specification have the following meaning.

The term “Calvin cycle” as used herein is understood as the process, genes and enzymes utilized by microorganisms and by plants to ensure carbon dioxide fixation. In this process, carbon dioxide and water are converted into organic compounds that are necessary for metabolic and cellular processes. There are various wild-type organisms that utilize a native Calvin cycle for producing organic compounds e.g., cyanobacteria, or purple bacteria or green bacteria. The Calvin cycle requires various enzymes to ensure proper regulation occurs and can be divided into three major phases: carbon fixation, reduction, and regeneration of ribulose. Each of these phases are tightly regulated and require unique and specific enzymes.

During the first phase of the Calvin cycle, carbon fixation occurs. The carbon dioxide is combined with ribulose 1,5-bisphosphate to form two 3-phosphoglycerate molecules. The enzyme that catalyzes this specific reaction is ribulose-bisphosphate carboxylase (RuBisCO). RuBisCO is the first enzyme utilized in the process of carbon fixation, which is capable of enzymatically processing its substrate, ribulose 1,5-bisphosphate.

During the second phase of the Calvin cycle, reduction occurs. The 3-phosphoglycerate molecules synthesized in phase 1 are reduced to glyceraldehyde-3-phosphate.

During the third phase of the Calvin cycle, regeneration of RuBisCO occurs. This specific phase involves a series of reactions in which there are a variety of enzymes required to ensure proper regulation. This phase is characterized by the conversion of 3-phosphoglycerate molecules, which was produced in earlier phase, back to ribulose 1,5-bisphosphate. The enzymes involved in this process include: triose phosphate isomerase, aldolase, fructose-1,6-bisphosphatase, transketolase, sedoheptulase-1,7-bisphosphatase, phosphopentose isomerase, phosphopentose epimerase, and phosphoribulokinase. The following is a brief summary of each enzyme and its role in the regeneration of ribulose 1,5-bisphosphate in the order it appears in this specific phase.

The key enzyme of the Calvin cycle is the ribulose-1,5-bisphosphate carboxylase/oxygenase (RuBisCO) complex which converts ribulose-1,5-diphosphate into two molecules of 3-phosphoglycerate by capturing a carbon dioxide molecule, and the ribulose phosphate kinase also called phosphoribulokinase, PRK).

Several forms of RuBisCO exist (Tabita et al., J Exp Bot, 59, 1515-24, 2008), of which the most represented are form I and form II. Form I consists of two types of subunits: large subunits (RbcL) and small subunits (RbcS). The functional enzyme complex is a hexadecamer made up of eight L subunits and eight S subunits. Correct assembly of these subunits further requires the intervention of at least one specific chaperone: RbcX (Liu et al., Nature, 463, 197-202, 2010). Form II is much simpler: it is a dimer formed of two identical RbcL subunits.

Form II RuBisCO enzyme can e.g. be obtained from recombinant microorganisms upon co-expressing the RuBisCO gene (e.g. of Thiobacillus denitrificans, SEQ ID NO:1) with chaperones, specifically with bacterial chaperones, e.g. GroES and GroEL.

Ribulose-1,5-diphosphate, the substrate of RuBisCO, is formed by reaction of ribulose-5-phosphate with ATP, catalyzed by PRK. Two classes of PRKs are known: class I enzymes, encountered in proteobacteria, are octamers, whereas those of class II, found in cyanobacteria and plants, are tetramers or dimers (Hariharan, T., Johnson, P. J., & Cattolica, R. A. (1998). Purification and characterization of phosphoribulokinase from the marine chromophytic alga Heterosigma carterae. Plant Physiology, 117(1), 321-9.) Form II PRK is encoded by the PRK gene, e.g. from Spinacia oleracea (SEQ ID NO:2).

There is no wild-type yeast which comprises RuBisCO and/or PRK, which is why yeasts are understood as non-autotrophic (or heterotrophic) organisms. However, the other Calvin cycle enzymes are present because they are used in other yeast metabolic processes.

Contrary to a native Calvin cycle which is present in photosynthetic organisms, yeasts can be engineered to express a functional Calvin cycle only as a synthetic Calvin cycle. The synthetic Calvin cycle is herein understood as a Calvin cycle, which utilizes heterologous genes encoding at least the RuBisCO and PRK enzymes. Such synthetic Calvin cycle is herein understood to be functional, if the carbon fixation pathway is active in the yeast (i.e. it utilizes carbon dioxide through the not naturally occurring or non-native, synthetic carbon fixation pathway) for the production of a carbohydrate which is used as a biomass precursor. As such, the heterologous genes described herein are expressed in a way that they are positioned relative to one another (e.g. in the same cellular compartment, such as the peroxisome or in a synthetic compartment similar to carboxysomes) such that they are able to function to cause carbon fixation. Functionality of the synthetic Calvin cycle can be tested as follows: Functionality of the proposed pathway can be verified in any engineered organism, which expresses all said heterologous enzymes, by growth on ¹³C labelled carbon dioxide as a carbon source. The ¹³C stemming from carbon dioxide is incorporated into biomass forming biomass precursor metabolites including 3-phosphoglycerate, glyceraldehyde 3-phosphate, dihydroxyacetone phosphate, ribulose-5-phosphate, ribose-5-phosphate, seduheptulose-1,7-bisphosphate and ribulose-1,5-bisphosphate. The ¹³C label can be measured following published LC-MS and GC-MS protocols (Rußmayer, H., Buchetics, M., Gruber, C., Valli, M., Grillitsch, K., Modarres, G., Gasser, B. (2015). Systems-level organization of yeast methylotrophic lifestyle. BMC Biology, 13(1), 80; Mairinger, T., Steiger, M., Nocon, J., Mattanovich, D., Koellensperger, G., Hann, S., 2015. GC-QTOFMS based determination of isotopologue and tandem mass isotopomer fractions of primary metabolites for ¹³C-metabolic flux analysis. Anal. Chem. acs.analchem.5b03173. doi:10.1021/acs.analchem.5b03173).

The term “carbon molecule” is herein understood as “carbon substrate” and shall mean a fermentable carbon substrate, typically a carbon source to produce organic carbon compounds, suitable as an energy source for microorganisms. C1 carbon sources are anorganic or organic compounds which comprise only one carbon atom per molecule or ion. Exemplary C1 carbon molecules used as substrates for biomass production and other fermentation processes described herein include natural gas, carbon dioxide (in the gaseous or solubilized form), carbon monoxide, methanol and synthesis gas (a mixture of carbon monoxide and hydrogen). The carbon source may be used as a single carbon source or as a mixture of different carbon sources.

The term “cell line” as used herein refers to an established clone of a particular cell type that has acquired the ability to proliferate over a prolonged period of time. The term “host cell line” refers to a cell line as used for expressing an endogenous or recombinant gene or genes of a metabolic pathway to produce polypeptides and cell metabolites mediated by such polypeptides, respectively. A cell line prepared for recombination with one or more heterologous genes to incorporate the genes into the cell genome, is herein also referred to as “chassis” cell line. A “production host cell line” or “production cell line” is commonly understood to be a cell line ready-to-use for cultivation/culturing in a bioreactor to obtain the product of a production process, such as a POI or metabolite. The yeast host or yeast cell line as described herein is particularly understood as a recombinant yeast organism, which may be cultivated/cultured to produce a POI or a host cell metabolite.

The term “cell culture” or “cultivation” (“culturing” is herein synonymously used), also termed “fermentation”, with respect to a host cell line is meant to be the maintenance of cells in an artificial, e.g., an in vitro environment, under conditions favoring growth, differentiation or continued viability, in an active or quiescent state, of the cells, specifically in a controlled bioreactor according to methods known in the industry. When cultivating, a cell culture is brought into contact with the cell culture media in a culture vessel or with substrate under conditions suitable to support cultivation of the cell culture. In certain embodiments, a culture medium as described herein is used to culture cells according to standard cell culture techniques that are well-known in the art. In some aspects, a culture medium is provided that can be used for the growth of yeast.

Cell culture media provide the nutrients necessary to maintain and grow cells in a controlled, artificial and in vitro environment. Characteristics and compositions of the cell culture media vary depending on the particular cellular requirements. Important parameters include osmolality, pH, and nutrient formulations. Feeding of nutrients may be done in a continuous or discontinuous mode according to methods known in the art. The culture media used in a method described herein are particularly useful for producing recombinant proteins.

Whereas a batch process is a cultivation mode in which all the nutrients necessary for cultivation of the cells are contained in the initial culture medium, without additional supply of further nutrients during fermentation, in a fed-batch process, after a batch phase, a feeding phase takes place in which one or more nutrients are supplied to the culture by feeding. The purpose of nutrient feeding is to increase the amount of biomass in order to increase the amount of recombinant protein as well.

In certain embodiments, the method described herein is a fed-batch process. Specifically, a host cell transformed with a nucleic acid construct encoding a desired recombinant POI or a metabolic pathway, is cultured in a growth phase medium and transitioned to a production phase medium in order to produce a desired recombinant POI or a cell metabolite.

In another embodiment, host cells described herein are cultivated in continuous mode, e.g. a chemostat. A continuous fermentation process is characterized by a defined, constant and continuous rate of feeding of fresh culture medium into the bioreactor, whereby culture broth is at the same time removed from the bioreactor at the same defined, constant and continuous removal rate. By keeping culture medium, feeding rate and removal rate at the same constant level, the cultivation parameters and conditions in the bioreactor remain constant.

A stable cell culture as described herein is specifically understood to refer to a cell culture maintaining the genetic properties, specifically keeping a POI or metabolite production level high, e.g. at least at a μg level, even after about 20 generations of cultivation, preferably at least 30 generations, more preferably at least 40 generations, most preferred of at least 50 generations. Specifically, a stable recombinant host cell line is provided which is considered a great advantage when used for industrial scale production.

The cell culture described herein is particularly advantageous for methods on an industrial manufacturing scale, e.g. with respect to both the volume and the technical system, in combination with a cultivation mode that is based on feeding of nutrients, in particular a fed-batch or batch process, or a continuous or semi-continuous process (e.g. chemostat).

The term “expression” or “expression system” or “expression cassette” is understood in the following way. Nucleic acid molecules containing a desired coding sequence and control sequences in operable linkage are used to transform or transfect hosts cells in order to express the coding sequence, thereby producing the encoded proteins or host cell metabolites. In order to effect transformation, the expression system may be included in a vector, e.g. a vector comprising a gene of interest encoding a POI. However, the relevant DNA may also be integrated into the host chromosome. Expression may refer to secreted or non-secreted expression products, including e.g., a POI or metabolites.

The terms “expression constructs” or “vectors” or “plasmid” used herein are defined as DNA sequences that are required for the transcription of cloned recombinant nucleotide sequences, i.e. of recombinant genes and the translation of their mRNA in a suitable host organism. Expression vectors or plasmids usually comprise an origin for autonomous replication in the host cells, selectable markers (e.g. an amino acid synthesis gene or a gene conferring resistance to antibiotics such as zeocin, kanamycin, G418 or hygromycin), a number of restriction enzyme cleavage sites, a suitable promoter sequence and a transcription terminator, which components are operably linked together. The terms “plasmid” and “vector” as used herein include autonomously replicating nucleotide sequences as well as genome integrating nucleotide sequences. A typical expression cassette includes in the direction of the 5′ end to the 3′ end of the nucleic acid molecule: promoter, one or more coding sequences, and a terminator.

The term “functional” as used herein e.g., in the context of an enzyme activity, shall refer to a functionally active molecule. A functional enzyme is specifically characterized by a catalytic center recognizing the enzyme substrate and catalysing the conversion of the substrate to a conversion product. Enzyme variants are considered functional upon determining their enzymatic activity in a standard test system, e.g. wherein the enzymatic activity is at least 50% of the activity of the parent (not modified or wild-type enzyme), or at least any of 60%, 70%, 80%, 90%, 100%, or even more than 100%.

The term “promoter” as used herein refers to a DNA sequence capable of controlling the expression of a coding sequence or functional RNA. Promoter activity may be assessed by its transcriptional efficiency. This may be determined directly by measurement of the amount of mRNA transcription from the promoter, e.g. by Northern Blotting or indirectly by measurement of the amount of gene product expressed from the promoter.

A “methanol-inducible promoter” is herein understood as a naturally occurring or wild-type promoter controlling expression of genes of the methanol dissimilatory pathway of organisms, in particular methylotrophic microorganisms.

According to the methanol dissimilatory pathway in methylotrophic yeast, such as P. pastoris, methanol passively diffuses into the yeast peroxisome. There it is converted to formaldehyde by one of two different alcohol oxidase isozymes (Aox1, Aox2). Formaldehyde can be further oxidized in several steps to CO₂ via the methanol dissimilatory pathway. Alternatively, formaldehyde is incorporated into the pentose phosphate pathway via a condensation reaction with xylulose 5-phosphate, a reaction catalyzed by a specialized transketolase enzyme called DiHydroxyAcetone Synthase (Das). This reaction yields a molecule of dihydroxyacetone (DHA) and a molecule of glyceraldehyde 3-phosphate. Each of these reactions occurs in peroxisomes in methylotrophic yeasts.

As an alternative to native or wild-type promoter sequences, functional variants of such native or wild-type promoter sequences (herein understood as parent promoters) can be used, which have at least 90% sequence identity and are functional in controlling the expression of a gene in substantially similar way, e.g. being an inducible promoter or constitutive promoter as the parent promoter.

The term “heterologous” as used herein with respect to a nucleotide or amino acid sequence or protein, refers to a compound which is either foreign, i.e. “exogenous” to a given host cell, such as not found in nature, or found in nature but in a different species; or that is naturally found in a given (wild-type) host cell, e.g., is “endogenous”, however, in the context of a heterologous construct, e.g. employing a heterologous nucleic acid. The heterologous nucleotide sequence as found endogenously may also be produced in an unnatural, e.g. greater than expected or greater than naturally found, amount in the cell, or in an unnatural compartment of the cell. The heterologous nucleotide sequence, or a nucleic acid comprising the heterologous nucleotide sequence, possibly differs in sequence from the endogenous nucleotide sequence but encodes the same protein as found endogenously. Specifically, heterologous nucleotide sequences are those not found in the same relationship to a host cell in nature. Any recombinant or artificial nucleotide sequence is understood to be heterologous. An example of a heterologous polynucleotide is a nucleotide sequence not natively associated with the promoter which controls expression of the coding nucleotide sequence.

As described herein, enzymes of a synthetic Calvin cycle may be heterologous, or encoded by a heterologous nucleic acid molecule or gene. The coding sequence may be operably linked to a promoter which is endogenous to the yeast host cell, or heterologous. Typically, the yeast is engineered to comprise a recombinant nucleotide sequence comprising a promoter and a coding sequence, which are not natively associated or not natively operably linked to each other.

As a further example of a heterologous compound is a POI encoding polynucleotide operably linked to a transcriptional control element, e.g., a promoter controlling the expression of the polynucleotide, or a termination signal sequence, to which the polynucleotide is not normally operably linked.

The heterologous carbon fixation enzymes to be expressed in a particular microorganism will vary according to the enzymes which are natively expressed in that microorganism, or which will need to be overexpressed for the improved function of the Calvin cycle. The heterologous genes introduced in a yeast host cell and expressed by the recombinant yeast, may be of any origin, e.g. of eukaryotic or prokaryotic organisms, artificial variants thereof, or synthetic ones.

Exemplary heterologous genes as described herein consist of naturally-occurring genes or polynucleotides, or those which are endogenous to the host cell, yet are artificially linked to the PTS as described herein. Such constructs are artificial constructs, which do not occur in nature, thus are synthetic or artificial.

A heterologous enzyme of the Calvin cycle described herein also refers to homologs and functional variants of wild-type enzymes, which are functional having the respective enzyme activity, including insertions, substitutions or deletions of one or more amino acids to the sequence (e.g., enzyme proteins which have at least 60%, or at least 70%, or at least 80%, or at least 90%, or at least 95% sequence identity to the native amino acid sequence of the enzyme, e.g., as determined using BlastP software of the National Center of Biotechnology Information (NCBI) using default parameters.

Exemplary RuBisCO may be encoded by a wild-type RuBisCO gene encoding a naturally occurring RuBisCO enzyme, or a codon-optimized polynucleotide encoding the naturally occurring RuBisCO enzyme. For example RuBisCO may be of bacterial origin, preferably of the genus Thiobacillus, Sideroxydans, Leptothrix, Methylobacillus, Sulfuritalea, Gallionellales, Rhodoferax, Rhodoferax, Burkholderiales, Thiomonas, Thiothrix, Halothiobacillus, Acidihalobacter, Limnohabitans, Acidithiobacillus, Lamprocystis, Thiocystis, Allochromatium or Thiorhodococcus. According to a specific example, RuBisCO is encoded by a RuBisCO gene of Thiobacillus denitrificans, Thiobacillus sp. 65-29, Thiobacillus sp. 65-1402, Thiobacillus thioparus, Thiobacillus sp. GWE1_62_9, Thiobacillus thiophilus, Thiobacillus sajanensis, Thiobacillus sp. 65-1059, Thiobacillus sp. SCN 63-374, Sideroxydans lithotrophicus, Sulfuritalea hydrogenivorans, Rhodoferax fermentans, Thiomonas intermedia, Halothiobacillus neapolitanus, Acidihalobacter prosperus, Acidithiobacillus caldus, Lamprocystis purpurea, Allochromatium warmingii or Thiorhodococcus drewsii origin, e.g. comprising the nucleotide sequence identified as SEQ ID NO:NO:1, or a functionally active variant thereof with at least 90% or 95% sequence identity expressing a functional ribulose-bisphosphate carboxylase.

Exemplary PRK may be encoded by a wild-type PRK gene encoding a naturally occurring PRK enzyme, or a codon-optimized polynucleotide encoding the naturally occurring PRK enzyme. For example PRK may be of plant origin, preferably of the family Amaranthaceae, Cucurbitaceae, Asteraceae, Apiaceae, Fabaceae, Salicaceae, Gesneriaceae, Poaceae, Brassicaceae, Zosteraceae, Ectocarpaceae or Malvaceae According to a specific example, PRK is encoded by a PRK gene of Spinacia oleracea origin, or of Beta vulgaris subsp. Vulgaris, Cucumis sativus, Cucumis melo, Helianthus annuus, Daucus carota subsp. sativus, Vigna angularis, Populus tomentosa, Dorcoceras hygrometricum, Triticum aestivum, Noccaea caerulescens, Brassica napus, Zostera marina, Zea mays, Ectocarpus siliculosus or Corchorus capsularis origin, e.g. comprising the nucleotide sequence identified as SEQ ID NO:2, or a functionally active variant thereof with at least 90% or 95% sequence identity expressing a functional ribulose phosphate kinase.

Exemplary PGK1 may be encoded by a wild-type PGK1 gene encoding a naturally occurring PGK1 enzyme, or a codon-optimized polynucleotide encoding the naturally occurring PGK1 enzyme. For example PGK1 may be of yeast origin, preferably of the genus Ogataea, Wickerhamomyces, Pichia, Cyberlindnera, Kuraishia, Cyberlindnera, Pachysolen, Meyerozyma, Brettanomyces, Babjeviella, Scheffersomyces, Hyphopichia, Schwanniomyces, Kluyveromyces, Hanseniaspora, Lachancea, Zygosaccharomyces, Eremothecium, Zygosaccharomyces, Hanseniaspora, Kazachstania, Saccharomyces, Komatagella, Yarrowia, Hansenula or Candida. According to a specific example, PGK1 is encoded by a PGK1 gene of Ogataea polymorpha origin, or of Ogataea parapolymorpha, Wickerhamomyces anomalus NRRL Y-366-8, Pichia kudriavzevii, Cyberlindnera fabianii, Kuraishia capsulata CBS 1993, Pachysolen tannophilus NRRL Y-2460, Meyerozyma guilliermondii ATCC 6260, Brettanomyces bruxellensis AWRI1499, Babjeviella inositovora NRRL Y-12698, Scheffersomyces stipitis CBS 6054, Schwanniomyces polymorphus, Kluyveromyces lactis, Hanseniaspora uvarum, Hanseniaspora guiffiermondii, Saccharomyces cerevisiae S288C, Klyveromyces marxianus, Komagataella pastoris, Komagataella phaffii, Yarrowia lipolytica, Candida boidinii or Candida albicans origin, e.g. comprising the nucleotide sequence identified as SEQ ID NO:3, or a functionally active variant thereof with at least 90% or 95% sequence identity expressing a functional phosphoglycerate kinase.

Exemplary TDH3 may be encoded by a wild-type TDH3 gene encoding a naturally occurring TDH3 enzyme, or a codon-optimized polynucleotide encoding the naturally occurring TDH3 enzyme. For example TDH3 may be of yeast origin, preferably of the genus Ogataea, Wickerhamomyces, Pichia, Cyberlindnera, Kuraishia, Cyberlindnera, Pachysolen, Meyerozyma, Brettanomyces, Babjeviella, Scheffersomyces, Hyphopichia, Schwanniomyces, Kluyveromyces, Hanseniaspora, Lachancea, Zygosaccharomyces, Eremothecium, Zygosaccharomyces, Hanseniaspora, Kazachstania, Saccharomyces, Komatagella, Yarrowia, Hansenula or Candida. According to a specific example, TDH3 is encoded by a TDH3 gene of Ogataea polymorpha origin, or of Ogataea parapolymorpha, Wickerhamomyces anomalus NRRL Y-366-8, Pichia kudriavzevii, Cyberlindnera fabianii, Kuraishia capsulata CBS 1993, Pachysolen tannophilus NRRL Y-2460, Meyerozyma guilliermondii ATCC 6260, Brettanomyces bruxellensis AWRI1499, Babjeviella inositovora NRRL Y-12698, Scheffersomyces stipitis CBS 6054, Schwanniomyces polymorphus, Kluyveromyces lactis, Hanseniaspora uvarum, Hanseniaspora guilliermondii, Saccharomyces cerevisiae 5288C, Klyveromyces marxianus, Komagataella pastoris, Komagataella phaffii, Yarrowia lipolytica, Candida boidinii or Candida albicans origin. e.g. comprising the nucleotide sequence identified as SEQ ID NO: 4, or a functionally active variant thereof with at least 90% or 95% sequence identity expressing a functional glyceraldehyde-3-phosphate dehydrogenase.

Exemplary TPI1 may be encoded by a wild-type TPI1 gene encoding a naturally occurring TPI1 enzyme, or a codon-optimized polynucleotide encoding the naturally occurring TPI1 enzyme. For example TPI1 may be of yeast origin, preferably of the genus Ogataea, Wickerhamomyces, Pichia, Cyberlindnera, Kuraishia, Cyberlindnera, Pachysolen, Meyerozyma, Brettanomyces, Babjeviella, Scheffersomyces, Hyphopichia, Schwanniomyces, Kluyveromyces, Hanseniaspora, Lachancea, Zygosaccharomyces, Eremothecium, Zygosaccharomyces, Hanseniaspora, Kazachstania, Saccharomyces, Komatagella, Yarrowia, Hansenula or Candida. According to a specific example, TPI1 is encoded by a TPI1 gene of Ogataea parapolymorpha origin, or of Ogataea polymorpha, Wickerhamomyces anomalus NRRL Y-366-8, Pichia kudriavzevii, Cyberlindnera fabianii, Kuraishia capsulata CBS 1993, Pachysolen tannophilus NRRL Y-2460, Meyerozyma guilliermondii ATCC 6260, Brettanomyces bruxellensis AWRI1499, Babjeviella inositovora NRRL Y-12698, Scheffersomyces stipitis CBS 6054, Schwanniomyces polymorphus, Kluyveromyces lactis, Hanseniaspora uvarum, Hanseniaspora guilliermondii, Saccharomyces cerevisiae S288C, Klyveromyces marxianus, Komagataella pastoris, Komagataella phaffii, Yarrowia lipolytica, Candida boidinii or Candida albicans origin, e.g. comprising the nucleotide sequence identified as SEQ ID NO: 5, or a functionally active variant thereof with at least 90% or 95% sequence identity expressing a functional triose-phosphate isomerase.

Exemplary TKL1 may be encoded by a wild-type TKL1 gene encoding a naturally occurring TKL1 enzyme, or a codon-optimized polynucleotide encoding the naturally occurring TKL1 enzyme. For example TKL1 may be of yeast origin, preferably of the genus Ogataea, Wickerhamomyces, Pichia, Cyberlindnera, Kuraishia, Cyberlindnera, Pachysolen, Meyerozyma, Brettanomyces, Babjeviella, Scheffersomyces, Hyphopichia, Schwanniomyces, Kluyveromyces, Hanseniaspora, Lachancea, Zygosaccharomyces, Eremothecium, Zygosaccharomyces, Hanseniaspora, Kazachstania, Saccharomyces, Komatagella, Yarrowia, Hansenula or Candida. According to a specific example, TKL1 is encoded by a TKL1 gene of Ogataea parapolymorpha origin, or of Ogataea polymorpha, Wickerhamomyces anomalus NRRL Y-366-8, Pichia kudriavzevii, Cyberlindnera fabianii, Kuraishia capsulata CBS 1993, Pachysolen tannophilus NRRL Y-2460, Meyerozyma guilliermondii ATCC 6260, Brettanomyces bruxellensis AWRI1499, Babjeviella inositovora NRRL Y-12698, Scheffersomyces stipitis CBS 6054, Schwanniomyces polymorphus, Kluyveromyces lactis, Hanseniaspora uvarum, Hanseniaspora guiffiermondii, Saccharomyces cerevisiae S288C, Klyveromyces marxianus, Komagataella pastoris, Komagataella phaffii, Yarrowia lipolytica, Candida boidinii or Candida albicans origin, e.g. comprising the nucleotide sequence identified as SEQ ID NO: 6, or a functionally active variant thereof with at least 90% or 95% sequence identity expressing a functional transketolase.

Exemplary chaperones may be encoded by genes which are heterologous or endogenous to the yeast host cell as described herein. Such chaperones are specifically functional as chaperones to support folding of a functional RuBisCO enzyme encoded by the RuBisCO gene.

GroEL may for example be encoded by a wild-type GroEL gene encoding a naturally occurring GroEL chaperone, or a codon-optimized polynucleotide encoding the naturally occurring GroEL chaperone. For example GroEL may be of bacterial origin, preferably of the genus Escherichia, Thiobacillus, Bacillus, Lactobacillus, Pseudomonas, Atlantibacter, Klebsiella, Pectcobacterium, Shimweffia, Franconibacter, Pantoea, Mangrovibacter, Nissabacter, Cronobacter, Rouxiella, Plesiomonas, Morganella or Yersinia. According to a specific example, GroEL is encoded by a GroEL gene of Escherichia coli origin, or Shigella flexneri, Atlantibacter hermannii, Klebsiella aerogenes, Shimweffia blattae, Enterobacter cloacae, Pantoea alhagi, Providencia stuartii, Moellerella wisconsensis, Thiobacillus denitrificans, Bacillus subtilis, Lactobacillus plantarum or Pseudomonas putida origin, e.g. comprising the nucleotide sequence identified as SEQ ID NO: 7, or a functionally active variant thereof with at least 90% or 95% sequence identity expressing a functional chaperone.

GroES may for example be encoded by a wild-type GroES gene encoding a naturally occurring GroES chaperone, or a codon-optimized polynucleotide encoding the naturally occurring GroES chaperone. For example GroES may be of bacterial origin, preferably of the genus Escherichia, Thiobacillus, Bacillus, Lactobacillus, Pseudomonas, Atlantibacter, Klebsiella, Pectcobacterium, Shimweffia, Franconibacter, Pantoea, Mangrovibacter, Nissabacter, Cronobacter, Rouxiella, Plesiomonas, Morganella or Yersinia. According to a specific example, GroES is encoded by a GroES gene of Escherichia coli origin, or Shigella flexneri, Atlantibacter hermannii, Klebsiella aerogenes, Shimwellia blattae, Enterobacter cloacae, Pantoea alhagi, Providencia stuartii, Moellerella wisconsensis, Thiobacillus denitrificans, Bacillus subtilis, Lactobacillus plantarum or Pseudomonas putida origin, e.g. comprising the nucleotide sequence identified as SEQ ID NO:8, or a functionally active variant thereof with at least 90% or 95% sequence identity expressing a functional chaperone.

The term “sequence identity” of a variant as compared to a parent sequence indicates the degree of identity (or homology) in that two or more nucleotide sequences have the same or conserved base pairs at a corresponding position, to a certain degree, up to a degree close to 100%. A homologous sequence typically has at least about 50% nucleotide sequence identity, preferably at least about 60% identity, more preferably at least about 70% identity, more preferably at least about 80% identity, more preferably at least about 90% identity, more preferably at least about 95% identity.

“Percent (%) amino acid sequence identity” with respect to polypeptide or protein sequences is defined as the percentage of amino acid residues in a candidate sequence that are identical with the amino acid residues in the specific polypeptide sequence, after aligning the sequence and introducing gaps, if necessary, to achieve the maximum percent sequence identity, and not considering any conservative substitutions as part of the sequence identity. Those skilled in the art can determine appropriate parameters for measuring alignment, including any algorithms needed to achieve maximal alignment over the full length of the sequences being compared.

“Percent (%) identity” with respect to the nucleotide sequence e.g., of a promoter or a gene, is defined as the percentage of nucleotides in a candidate DNA sequence that is identical with the nucleotides in the DNA sequence, after aligning the sequence and introducing gaps, if necessary, to achieve the maximum percent sequence identity, and not considering any conservative substitutions as part of the sequence identity. Alignment for purposes of determining percent nucleotide sequence identity can be achieved in various ways that are within the skill in the art, for instance, using publicly available computer software. Those skilled in the art can determine appropriate parameters for measuring alignment, including any algorithms needed to achieve maximal alignment over the full length of the sequences being compared.

For purposes described herein, the sequence identity between two sequences is determined using the NCBI BLAST program version 2.2.29 (Jan. 6, 2014) with blastn or blastp set at the following exemplary parameters: Word Size: 11; Expect value: 10; Gap costs: Existence=5, Extension=2; Filter=low complexity activated; Match/Mismatch Scores: 2, −3; Filter String: L; m.

The term “metabolite” as used herein shall refer to products of metabolic reactions catalyzed by enzymes of a cell metabolic pathway or pathways and include reactant, product and cofactor molecules of said enzymes. Metabolites may arise in the same pathway(s) as the cell metabolic pathway or pathways encoding an enzyme which catalyzes the synthesis of the cell growth and/or productivity inhibitor or intermediate thereof or may be synthesized in a branching pathway.

The term “operably linked” as used herein refers to the association of nucleotide sequences on a single nucleic acid molecule, in a way such that the function of one or more nucleotide sequences is affected by at least one other nucleotide sequence present on said nucleic acid molecule. For example, a promoter is operably linked with a coding sequence of a recombinant gene, when it is capable of effecting the expression of that coding sequence. As a further example, a nucleic acid encoding a signal peptide is operably linked to a nucleic acid sequence encoding a POI, when it is capable of expressing a protein in the secreted form, such as a preform of a mature protein or the mature protein. Specifically, such nucleic acids operably linked to each other may be immediately linked, i.e. without further elements or nucleic acid sequences in between the nucleic acid encoding a signal peptide and the nucleic acid sequence encoding a POI.

A promoter sequence is typically understood to be operably linked to a coding sequence, if the promoter controls the transcription of the coding sequence. If a promoter sequence is not natively associated with the coding sequence, its transcription is either not controlled by the promoter in native (wild-type) cells or the sequences are recombined with different contiguous sequences.

The term “peroxisomal targeting signal” (PTS) as used herein shall refer to short nucleic acid sequences which when linked to or positioned within a coding sequence, e.g. as a nucleotide sequence encoding a C-terminal tripeptide or an N-terminal peptide of 5-9 amino acids, directs the expression of the expression product to the peroxisome of the host cell. By such a functional PTS, an enzyme can be relocated to the peroxisome. Most organism including Pichia pastoris have two different targeting systems. The first one (PTS1) uses the receptor Pex5 to achieve targeting to the peroxisome. The second one (PTS2) uses Pex7 as receptor. A functional PTS is an amino acid sequence which is specifically recognized by any of the receptors Pex 5 (PTS1) or Pex7 (PTS2), thereby activating the receptor and directing expression of the gene that is fused with such PTS to the host cell peroxisome.

A nucleotide sequence encoding the PTS1 is typically linked to a gene at the 3′-end, such that the PTS is fused at the carboxy terminus of the respective gene expression product. Thereby, the C-terminus of the amino acid sequence of the gene expression product is directly linked to the N-terminus of the PTS.

A nucleotide sequence encoding the PTS2 is typically linked to a gene at the 5′-end or integrated in proximity to the 5′-end, such that the PTS is fused at the amino terminus or close to the amino terminus of the respective gene expression product. Thereby, the N-terminus of the amino acid sequence of the gene expression product is directly linked to the C-terminus of the PTS2.

The following tools can be used to determine targeting signals in a given protein sequence: PTS1 predictor (Neuberger G, Maurer-Stroh S, Eisenhaber B, Hartig A, Eisenhaber F. Motif refinement of the peroxisomal targeting signal 1 and evaluation of taxon-specific differences. J Mol Biol. 2003 May 2; 328(3):567-79), or PTS prediction tool WoLF PSORT (Horton P, Park K-J, Obayashi T et al. WoLF PSORT: protein localization predictor. Nucleic Acids Res 2007; 35:W585-7).

The term “protein of interest” (POI) as used herein refers to a polypeptide or a protein that is produced by means of recombinant technology in a host cell. More specifically, the protein may either be a polypeptide not naturally occurring in the host cell, i.e. a heterologous protein, or else may be native to the host cell, i.e. a homologous protein to the host cell, but is produced, for example, by transformation with a self-replicating vector containing the nucleic acid sequence encoding the POI, or upon integration by recombinant techniques of one or more copies of the nucleic acid sequence encoding the POI into the genome of the host cell, or by recombinant modification of one or more regulatory sequences controlling the expression of the gene encoding the POI, e.g. of a promoter sequence.

The POI can be any eukaryotic, prokaryotic or synthetic polypeptide. Specifically, it can be a mammalian protein, including human or animal proteins. It can be a secreted protein or an intracellular protein. A POI can be a naturally occurring protein, or an artificial protein. The present methods and yeast host cells are also provided for the recombinant production of functional variants, derivatives or biologically active fragments of naturally occurring proteins.

A POI referred to herein may be a product homologous (or allogenic) to the eukaryotic host cell or a heterologous one, and is preferably prepared for therapeutic, prophylactic, diagnostic, analytic or industrial use.

The POI is preferably a heterologous recombinant polypeptide or protein, produced in a yeast cell, preferably as secreted proteins. Examples of preferably produced proteins are immunoglobulins, immunoglobulin fragments, aprotinin, tissue factor pathway inhibitor or other protease inhibitors, and insulin or insulin precursors, insulin analogues, growth hormones, interleukins, tissue plasminogen activator, transforming growth factor a or b, glucagon, glucagon-like peptide 1 (GLP-1), glucagon-like peptide 2 (GLP-2), GRPP, Factor VII, Factor VIII, Factor XIII, platelet-derived growth factor1, serum albumin, enzymes, such as lipases or proteases, or any of the groups of hydrolytic enzymes, transferases, oxidoreductases, lyases, isomerases, or ligases, or a functional homolog, functional equivalent variant, derivative and biologically active fragment with a similar function as the native protein. The POI may be structurally similar to the native protein and may be derived from the native protein by addition of one or more amino acids to either or both the C- and N-terminal end or the side-chain of the native protein, substitution of one or more amino acids at one or a number of different sites in the native amino acid sequence, deletion of one or more amino acids at either or both ends of the native protein or at one or several sites in the amino acid sequence, or insertion of one or more amino acids at one or more sites in the native amino acid sequence. Such modifications are well known for several of the proteins mentioned above.

A POI can also be selected from substrates, enzymes, inhibitors or cofactors that provide for biochemical reactions in the host cell, with the aim to obtain the product of said biochemical reaction or a cascade of several reactions, e.g. to obtain a metabolite of the host cell. Exemplary products can be vitamins, such as riboflavin, organic acids, and alcohols, which can be obtained with increased yields following the expression of a recombinant protein or a POI described herein.

The term “recombinant” as used herein shall mean “being prepared by or the result of genetic engineering”. Thus, a “recombinant microorganism” comprises at least one “recombinant nucleic acid”. The yeast described herein is understood as a recombinant yeast. A recombinant microorganism may comprise an expression vector or cloning vector, or it has been genetically engineered to contain a recombinant nucleic acid sequence.

A “recombinant protein” is produced by expressing a respective recombinant nucleic acid in a host. A “recombinant promoter” is a genetically engineered non-coding nucleotide sequence suitable for its use as a functionally active promoter as described herein.

In general, the recombinant nucleic acids or organisms as referred to herein may be produced by recombination techniques well known to a person skilled in the art. In accordance with the present invention there may be employed conventional molecular biology, microbiology, and recombinant DNA techniques within the skill of the art. Such techniques are explained fully in the literature. See, e.g., Maniatis, Fritsch & Sambrook, “Molecular Cloning: A Laboratory Manual, Cold Spring Harbor, (1982).

According to a specific embodiment described herein, a recombinant construct is prepared by ligating a promoter and relevant gene(s) encoding a POI into a vector or expression construct. The gene(s) can be stably integrated into the host cell genome by transforming the host cell using such vectors or expression constructs.

Expression vectors may include but are not limited to cloning vectors, modified cloning vectors and specifically designed plasmids. Any expression vector suitable for expression of a recombinant gene in a host cell can be used. Such vectors are typically selected depending on the host organism.

Appropriate expression vectors typically comprise further regulatory sequences suitable for expressing DNA encoding a POI in a yeast host cell. Examples of regulatory sequences include operators, enhancers, ribosomal binding sites, and sequences that control transcription and translation initiation and termination. The regulatory sequences may be operably linked to the DNA sequence to be expressed.

To allow expression of a recombinant nucleotide sequence in a host cell, the expression vector may provide the promoter adjacent to the 5′ end of the coding sequence, e.g. upstream from a gene of interest or a signal peptide gene enabling secretion of a POI. The transcription is thereby regulated and initiated by this promoter sequence.

The term “signal peptide” as used herein shall specifically refer to a native signal peptide, a heterologous signal peptide or a hybrid of a native and a heterologous signal peptide, and may specifically be heterologous or homologous to the host organism producing a POI. The function of the signal peptide is to allow the POI to be secreted to enter the endoplasmic reticulum. It is usually a short (3-60 amino acids long) peptide chain that directs the transport of a protein outside the plasma membrane, thereby making it easy to separate and purify a heterologous protein. Some signal peptides are cleaved from the protein by signal peptidase after the proteins are transported.

Exemplary signal peptides are signal sequences from S. cerevisiae alpha-mating factor prepro peptide and the signal peptides from the P. pastoris acid phosphatase gene (PHO1) and the extracellular protein X (EPX1) (WO2014067926A1).

Transformants as described herein can be obtained by introducing an expression vector DNA, e.g. plasmid DNA, into a host and selecting transformants which express a POI or the host cell metabolite with high yields. Host cells are treated to enable them to incorporate foreign DNA by methods conventionally used for transformation of eukaryotic cells, such as the electric pulse method, the protoplast method, the lithium acetate method, and modified methods thereof. P. pastoris is preferably transformed by electroporation. Preferred methods of transformation for the uptake of the recombinant DNA fragment by the microorganism include chemical transformation, electroporation or transformation by protoplastation. Transformants described herein can be obtained by introducing such a vector DNA, e.g. plasmid DNA, into a host and selecting transformants which express the relevant protein or host cell metabolite with high yields.

A cell culture product can be produced by culturing the recombinant host cell line in an appropriate medium, isolating the expressed POI or metabolite from the culture, and optionally purifying it by a suitable method.

Several different approaches for the production of the POI described herein are preferred. Substances may be expressed, processed and optionally secreted by transforming the yeast host cell with an expression vector harboring recombinant DNA encoding a relevant protein and at least one of the regulatory elements as described herein, preparing a culture of the transformed cell, growing the culture, inducing transcription and POI production, and recovering the product of the fermentation process.

The host cell described herein is specifically tested for its expression capacity or yield by the following test: ELISA, activity assay, HPLC, or other suitable tests.

The invention specifically allows for the fermentation process on a pilot or industrial scale. The industrial process scale would preferably employ volumina of at least 10 L, specifically at least 50 L, preferably at least 1 m³, preferably at least 10 m³, most preferably at least 100 m³.

Production conditions in industrial scale are preferred, which refer to e.g. fed batch cultivation in reactor volumes of 100 L to 10 m³ or larger, employing typical process times of several days, or continuous processes in fermenter volumes of approximately 50-1000 L or larger, with dilution rates of approximately 0.02-0.15 h⁻¹.

The suitable cultivation techniques may encompass cultivation in a bioreactor starting with a batch phase, followed by a short exponential fed batch phase at high specific growth rate, further followed by a fed batch phase at a low specific growth rate. Another suitable cultivation technique may encompass a batch phase followed by a continuous cultivation phase at a low dilution rate.

A transformant yeast described herein that is transformed with regulatory elements and/or POI encoding genes may preferably first be cultivated at conditions to grow efficiently to a large cell number, using carbon fixation. When the cell line is then cultivated for high yield POI production, cultivation techniques are chosen to produce the expression product.

A preferred embodiment includes a batch culture to provide biomass followed by a fed-batch culture for high yield POI production.

It is preferred to cultivate the host cell line as described herein in a bioreactor under growth conditions to obtain a cell density of at least 1 g/L cell dry weight, more preferably at least 10 g/L cell dry weight, preferably at least 20 g/L cell dry weight. It is advantageous to provide for such yields of biomass production on a pilot or industrial scale.

A growth medium allowing the accumulation of biomass as described herein, specifically a basal growth medium, typically comprises no or a limited amount of a carbon source, a nitrogen source, a source for sulphur and a source for phosphate. Typically, such a medium comprises furthermore trace elements and vitamins, and may further comprise amino acids, peptone or yeast extract.

Preferred nitrogen sources include NH₄H₂PO₄, or NH₃ or (NH₄)₂SO₄;

Preferred sulphur sources include MgSO₄, or (NH₄)₂SO₄ or K₂SO₄;

Preferred phosphate sources include NH₄H₂PO₄, or H₃PO₄ or NaH₂PO₄, KH₂PO₄, Na₂HPO₄ or K₂HPO₄;

Further typical medium components include KCl, CaCl₂, and Trace elements such as: Fe, Co, Cu, Ni, Zn, Mo, Mn, I, B;

Preferably the medium is supplemented with vitamin B₇;

A typical growth medium for yeast, in particular P. pastoris expressing a functional Calvin cycle as described herein, comprises only a limited amount of a carbon source like carbon dioxide, carbonate, methanol, glycerol, sorbitol or glucose. The limited amount is preferably at least 10 mg/L, preferably at least 100 mg/L, most preferred at least 1 g/L.

In the production phase a production medium is specifically used with only a limited amount of a supplemental carbon source. The limited amount is preferably at least 10 mg/L, preferably at least 100 mg/L, most preferred at least 1 g/L. A typical production medium for yeast, in particular P. pastoris expressing a functional Calvin cycle as described herein, comprises a utilizable carbon source (e.g. C1 carbon source, but also glucose, glycerol, sorbitol or methanol).

The fermentation preferably is carried out at a pH ranging from 3 to 7.5.

Typical fermentation times are about 24 to 120 hours with temperatures in the range of 20° C. to 35° C., preferably 22-30° C.

Specifically, the cells are cultivated under conditions suitable to effect expression of the desired POI or metabolite, which can be purified from the cells or culture medium, depending on the nature of the expression system and the expressed protein, e.g. whether the protein is fused to a signal peptide and whether the protein is soluble or membrane-bound. As will be understood by the skilled artisan, cultivation conditions will vary according to factors that include the type of host cell and particular expression vector employed.

A POI is preferably expressed employing conditions to produce yields of at least 1 mg/L, preferably at least 10 mg/L, preferably at least 100 mg/L, most preferred at least 1 g/L.

A metabolite is preferably expressed employing conditions to produce yields of at least 1 mg/L, preferably at least 10 mg/L, preferably at least 100 mg/L, most preferred at least 1 g/L.

It is understood that the methods disclosed herein may further include cultivating said recombinant host cells under conditions permitting the expression of the POI, either in the secreted form or else as intracellular product. A recombinant POI or a host cell metabolite can then be isolated from the cell culture medium and further purified by techniques well known to a person skilled in the art.

The POI produced according to a method described herein typically can be isolated and purified using state of the art techniques, including the increase of the concentration of the desired POI and/or the decrease of the concentration of at least one impurity.

Secretion of the recombinant expression products from the host cells is generally advantageous for reasons that include facilitating the purification process, since the products are recovered from the culture supernatant rather than from the complex mixture of proteins that results when yeast cells are disrupted to release intracellular proteins.

The cultured transformant cells may also be ruptured sonically or mechanically, enzymatically or chemically to obtain a cell extract containing the desired POI, from which the POI is isolated and purified.

As isolation and purification methods for obtaining a recombinant polypeptide or protein product, methods, such as methods utilizing difference in solubility, such as salting out and solvent precipitation, methods utilizing difference in molecular weight, such as ultrafiltration and gel electrophoresis, methods utilizing difference in electric charge, such as ion-exchange chromatography, methods utilizing specific affinity, such as affinity chromatography, methods utilizing difference in hydrophobicity, such as reverse phase high performance liquid chromatography, and methods utilizing difference in isoelectric point, such as isoelectric focusing may be used.

The highly purified product is essentially free from contaminating proteins, and preferably has a purity of at least 90%, more preferred at least 95%, or even at least 98%, up to 100%. The purified products may be obtained by purification of the cell culture supernatant or else from cellular debris.

As isolation and purification methods the following standard methods are preferred: Cell disruption (if the POI is obtained intracellularly), cell (debris) separation and wash by Microfiltration or Tangential Flow Filter (TFF) or centrifugation, POI purification by precipitation or heat treatment, POI activation by enzymatic digest, POI purification by chromatography, such as ion exchange (IEX), hydrophobic interaction chromatography (HIC), Affinity chromatography, size exclusion (SEC) or HPLC Chromatography, POI precipitation of concentration and washing by ultrafiltration steps.

The isolated and purified POI or metabolite can be identified by conventional methods such as Western blot, HPLC, activity assay, or ELISA.

The preferred yeast host cells are derived from methylotrophic yeast, such as from Pichia or Komagataella, e.g. Pichia pastoris, or Komagataella pastoris, or K. phaffii, or K. pseudopastoris. Examples of the host include yeasts such as P. pastoris. Examples of P. pastoris strains include CBS 704 (=NRRL Y-1603=DSMZ 70382), CBS 2612 (=NRRL Y-7556), CBS 7435 (=NRRL Y-11430), CBS 9173-9189 (CBS strains: CBS-KNAW Fungal Biodiversity Centre, Centraalbureau voor Schimmelcultures, Utrecht, The Netherlands), and DSMZ 70877 (German Collection of Microorganisms and Cell Cultures), but also strains from Invitrogen, such as X-33, GS115, KM71 and SMD1168. Examples of S. cerevisiae strains include W303, CEN.PK and the BY-series (EUROSCARF collection). All of the strains described above have been successfully used to produce transformants and express heterologous genes.

A preferred yeast host cell described herein, such as a P. pastoris or S. cerevisiae host cell, contains heterologous or recombinant promoter sequences, which may be derived from a P. pastoris or S. cerevisiae strain, different from the production host. In another specific embodiment the host cell described herein comprises a recombinant expression construct described herein comprising the promoter originating from the same genus, species or strain as the host cell.

If the POI is a protein homologous to the host cell, i.e. a protein which is naturally occurring in the host cell, the expression of the POI in the host cell may be modulated by the exchange of its native promoter sequence with a heterologous promoter sequence.

According to a specific embodiment, the POI production method employs a recombinant nucleotide sequence encoding the POI, which is provided on a plasmid suitable for integration into the genome of the host cell, in a single copy or in multiple copies per cell. The recombinant nucleotide sequence encoding the POI may also be provided on an autonomously replicating plasmid in a single copy or in multiple copies per cell.

The preferred method as described herein employs a plasmid, which is a eukaryotic expression vector, preferably a yeast expression vector. Expression vectors may include but are not limited to cloning vectors, modified cloning vectors and specifically designed plasmids. A preferred expression vector as used in a method described herein may be any expression vector suitable for expression of a recombinant gene in a host cell and is selected depending on the host organism. The recombinant expression vector may be any vector which is capable of replicating in or integrating into the genome of the host organisms, also called host vector, such as a yeast vector, which carries a DNA construct as described herein. A preferred yeast expression vector is for expression in yeast selected from the group consisting of methylotrophic yeasts represented by the genera Ogataea, Hansenula, Pichia, Candida and Torulopsis.

Specifically, plasmids derived from pPICZ, pGAPZ, pPIC9, pPICZalfa, pGAPZalfa, pPIC9K, pGAPHis or pPUZZLE are used as a vector.

According to a preferred embodiment, a recombinant construct is obtained by ligating the relevant genes into a vector. These genes can be stably integrated into the host cell genome by transforming the host cell using such vectors. The polypeptides encoded by the genes can be produced using the recombinant host cell line by culturing a transformant, thus obtained in an appropriate medium, isolating the expressed POI from the culture, and purifying it by a method appropriate for the expressed product, in particular to separate the POI from contaminating proteins.

Expression vectors may comprise one or more phenotypic selectable markers, e.g. a gene encoding a protein that confers antibiotic resistance or that supplies an autotrophic requirement. Yeast vectors commonly contain an origin of replication from a yeast plasmid, an autonomously replicating sequence (ARS), or alternatively, a sequence used for integration into the host genome, a promoter region, sequences for polyadenylation, sequences for transcription termination, and a selectable marker.

The procedures used to ligate the DNA sequences, regulatory elements and the gene(s) coding for the POI, the promoter and the terminator, respectively, and to insert them into suitable vectors containing the information necessary for integration or host replication, are well-known to persons skilled in the art, e.g. described by J. Sambrook et al., (A Laboratory Manual, Cold Spring Harbor, 1989).

Also multicloning vectors, which are vectors having a multicloning site, can be used, wherein a desired heterologous gene can be incorporated at a multicloning site to provide an expression vector. In expression vectors, the promoter is placed upstream of the gene of the POI and regulates the expression of the gene. In the case of multicloning vectors, because the gene of the POI is introduced at the multicloning site, the promoter is placed upstream of the multicloning site.

The DNA construct as provided to obtain a recombinant host cell may be prepared synthetically by established standard methods, e.g. the phosphoramidite method. The DNA construct may also be of genomic or cDNA origin, for instance obtained by preparing a genomic or cDNA library and screening for DNA sequences coding for all or part of the polypeptide by hybridization using synthetic oligonucleotide probes in accordance with standard techniques (Sambrook et al., Molecular Cloning: A Laboratory Manual, Cold Spring Harbor, 1989). Finally, the DNA construct may be of mixed synthetic and genomic, mixed synthetic and cDNA or mixed genomic and cDNA origin prepared by annealing fragments of synthetic, genomic or cDNA origin, as appropriate, the fragments corresponding to various parts of the entire DNA construct, in accordance with standard techniques.

In another preferred embodiment, the yeast expression vector is able to stably integrate in the yeast genome, e. g. by homologous recombination.

The foregoing description will be more fully understood with reference to the following examples. Such examples are, however, merely representative of methods of practicing one or more embodiments of the present invention and should not be read as limiting the scope of invention.

### EXAMPLES

In the following examples it is shown how a Pichia pastoris strain can be created, which contains a functional Calvin cycle targeted to the peroxisome or expressed in the cytosol. In example 2 the DNA construction part is explained and in example 3 the Pichia pastoris strain construction and screening is described. The media compositions used to cultivate and propagate the cells are described in Example 1. The main strain containing a fully functional Calvin cycle targeted to the peroxisome has the identifier “GaT_pp_10” and has the following genotype: Δ(aox1)₁(das1)₂(das2)₃::(TDH3, PRK, PGK1)₁(RuBisCO, GroEL, GroES)₂(TKL1, TPI1)₃

In example 4 it is shown that this strain (GaT_pp_10) can grow in the presence of methanol and carbon dioxide, whereas the control strains (GaT_pp_12, GaT_pp_13), which are missing parts of the Calvin cycle, cannot grow. This shows that this strain expresses a functional Calvin cycle.

In example 5, it is further shown that growth of GaT_pp_10 is dependent on the carbon source CO₂. In the presence of only methanol as an energy source, no growth is observed. It is also shown by this example that growth in the engineered strains is also possible without co-expression of the molecular chaperones GroEL and GroES.

In further examples it is outlined how valuable products like metabolites (lactic acid, example 6) or proteins (carboxypeptidase B or human serum albumin, example 7) can be produced with a P. pastoris strain containing a Calvin cycle. Example 8 is dedicated to show the impact of native P. pastoris Aox1, Das1 and Das2 on strains expressing a functional Calvin cycle. Finally, in example 9 a ¹³C labelling strategy is shown to provide further evidence for the carbon dioxide fixating capability of the strain GaT_pp_10.

In example 10, it is explained how a strain expressing a Calvin cycle in the cytosol was engineered. This strain has the unique identifier GaT_pp_22 and has the following genotype:

Δ(aox1)₁(das1)₂(das2)₃::(cTDH3, cPRK, cPGK1)₁(cRuBisCO, GroEL, GroES)₂(cTKL1, cTPI1)₃

In example 11, it is shown that this strain (GaT_pp_22) can grow in the presence of methanol and carbon dioxide, which demonstrates the functionality of the cytosolically expressed synthetic Calvin cycle. In examples 12 and 13 it is shown, how value-added chemicals (lactic acid, example 12 and itaconic acid, example 13) can be produced on CO₂ and methanol by GaT_pp_22 strains. Further, it is outlined how proteins (carboxypeptidase B or human serum albumin, example 14) can be produced in strains expressing a cytosolic Calvin cycle.

### Example 1 Media Preparation

LB medium was used for Escherichia coli DH 10B cultivations and the procedure is described in the following.

LB medium (10.0 g*L⁻¹ soy peptone (Quest), 5.0 g*L⁻¹ yeast extract (MERCK) and 5.0 g*L⁻¹ NaCl adjusted to pH=7.4-7.6 with 4N NaOH) was prepared and aliquoted in 500 mL Schott bottles. Sterilization was done by autoclaving at 121° C. for 20 min.

Yeast peptone (YP) medium was used for cultivations of Pichia pastoris CBS7435 wt in shake flasks and the procedure was as follows.

YP-medium (20.0 g*L⁻¹ soy peptone (Quest), 10.0 g*L⁻¹ yeast extract (MERCK) adjusted to pH=7.4-7.6 with 4N NaOH) was autoclaved prior to the addition of the carbon source. A ten times glucose stock (220 g*L⁻¹ D(+)-Glucose Monohydrate) was prepared and sterilized by autoclaving. The ten times glucose stock was added to YP medium in a 1/10 ratio resulting in YPD medium.

For bioreactor cultivations a glycerol containing batch medium (BatchGly) was prepared as follows.

The BatchGly was prepared according to Table 1. The pH (4.9-5.1) of the glycerol containing batch medium was adjusted with HCl (25%) and sterilization was performed by filtration (0.22 μm filter unit) into autoclaved glass bottles. The biotin solution was prepared with d-biotin (0.2 g*L⁻¹ in RO—H₂O) and complete dissolution was ensured by stirring under heating to 55-60° C. followed by sterile filtration (0.22 μm filter unit). The trace element solution was prepared according to Table 3.

Preparation of Labelling Medium (LM) was done according to Table 2. After preparation, the medium was sterile filtered (0.22 μm filter unit). The pH was adjusted in the bioreactors using 25% NH₃

TABLE 1
Composition of batch medium containing glycerol
(BatchGly) as a carbon source with supplier information.

| | | Concentration |
|---|---|---|
| Compound | Supplier | [g * L⁻¹] |
| Citrate monohydrate | ROTH | 2 |
| Glycerol | ROTH | 16 |
| (NH₄)₂HPO₄ | Applichem | 12.6 |
| MgSO₄*7 H₂O | ROTH | 0.5 |
| KCl | MERCK | 0.9 |
| CaCl₂*2 H₂O | ROTH | 0.022 |
| Trace element solution | sp | N/A |
| Biotin solution (0.2 g * L⁻¹) | MERCK | 0.0004 |

Trace element solution was self-prepared (sp) according to Table 2

TABLE 2
Composition of trace element solution

| | | Concentration |
|---|---|---|
| Compound | Supplier | [g * L⁻¹] |
| H₂SO₄ (95-98%) | MERCK | 0.01 |
| FeSO₄*7 H₂O | ROTH | 65 |
| ZnCl₂ | Applichem | 20 |
| CuSO₄*5 H₂O | MERCK | 6 |
| MnSO₄*H₂O | Riedel de Haen | 3.36 |
| CoCl₂*6 H₂O | MERCK | 0.82 |
| Na₂MoO₄*2 H₂O | MERCK | 0.2 |
| NaI | MERCK | 0.08 |
| H₃BO₃ | MERCK | 0.02 |

TABLE 3
Composition of trace element solution

| | | Concentration |
|---|---|---|
| Compound | Supplier | [g * L⁻¹] |
| H₂SO₄ (95-98%) | MERCK | 0.01 |
| FeSO₄*7 H₂O | ROTH | 65 |
| ZnCl₂ | Applichem | 20 |
| CuSO₄*5 H₂O | MERCK | 6 |
| MnSO₄*H₂O | Riedel de Haen | 3.36 |
| CoCl₂*6 H₂O | MERCK | 0.82 |
| Na₂MoO₄*2 H₂O | MERCK | 0.2 |
| NaI | MERCK | 0.08 |
| H₃BO₃ | MERCK | 0.02 |

For testing the engineered strains as production hosts, Yeast Nitrogen Base (YNB) medium was prepared (final concentration in Table 4).

TABLE 4
Composition of Yeast Nitrogen Base (YNB) medium

| | | Concentration |
|---|---|---|
| Compound | Supplier | [g * L⁻¹] |
| Yeast Nitrogen Base | Difco | 3.4 |
| (NH₄)₂SO₄ | MERCK | 10.0 |
| methanol | ROTH | 4.0 |

### Example 2 Plasmid and Linear DNA Fragment Construction

All expression cassettes (promoter, CDS, terminator) were constructed by Golden Gate cloning (Engler et al. PloS One 4 (5): e5553. doi:10.1371/journal.pone.0005553) and flanked with the respective integration sites to replace the three aforementioned genes, aox1, das1 and das2. The cloning workflow used for construction of all linear DNA fragments and guide RNA (gRNA)/hCas9 plasmids was achieved following the workflow for plasmid DNA construction published in (Sarkari, et al. 2017 Bioresource Technology. doi:10.1016/j.biortech.2017.05.004).

The coding sequences (CDS) of the genes mentioned in Table 5 were combined with methanol inducible promoters and terminator sequences from Pichia pastoris CBS7435 wt (Table 6).

TABLE 5
Genes required for the creation of the synthetic Calvin cycle in Pichia
pastoris (Centraalbureau voor Schimmelcultures, NL, genome sequenced by (Küberl
et al., 2011; Valli et al., 2016) with gene source, according enzymatic nomenclature
and EC number. C-terminal protein sequences were engineered to contain a
peroxisome targeting signal (PTS) by addition of 9 nucleotides at the 3′ end of each
CDS encoding for the tri-peptide SKL. Targeting was evaluated in silico by using the
PTS predictor tool provided by the Research Institute of Molecular Pathology (IMP),
Vienna (Neuberger et al. 2003, Journal of Molecular Biology; doi.org/10.1016/S0022-
2836(03)00319-X)

| | SEQ | | | EC | | PTS |
|---|---|---|---|---|---|---|
| Gene Name | ID NO | UniProt* | Source | Number | Full Name | added |
| cbbM- | 1 | Q60028 | Thiobacillus | 4.1.1.39 | Ribulose-bisphosphate | YES |
| RuBisCO | | | denitrificans (ATCC | | carboxylase | |
| | | | 25259) | | | |
| PRK | 2 | P09559.1 | Spinacia oleracea | 2.7.1.19 | Phosphoribulokinase | YES |
| PGK1 | 3 | A0A1B7SCV2 | Ogataea | 2.7.2.3 | Phosphoglycerate kinase | YES |
| | | | polymporpha (CBS | | | |
| | | | 4732) | | | |
| TDH3 | 4 | A0A1B7SCG5 | Ogataea | 1.2.1.12 | Glyceraldehyde-3- | YES |
| | | | polymporpha (CBS | | phosphate | |
| | | | 4732) | | dehydrogenase | |
| TPI1 | 5 | W1Q838 | Ogataea | 5.3.1.1 | Triosephosphate | YES |
| | | | parapolymorpha | | isomerase | |
| | | | (CBS11895) | | | |
| TKL1 | 6 | W1QKQ2 | Ogataea | 2.2.1.1 | Transketolase | YES |
| | | | parapolymorpha | | | |
| | | | (CBS11895) | | | |
| GroEL | 7 | B1XDP7 | Escherichia coli | N/A | molecular chaperone | NO |
| | | | (DH10B) | | GroEL | |
| GroES | 8 | B1XDP6 | Escherichia coli | N/A | molecular chaperone | NO |
| | | | (DH10B) | | GroES | |

*Uniprot: Universal Protein Resource

TABLE 6
Gene regulation elements (promoters P_{XXX} and terminators T_{XXX}) in
proposed synthetic Calvin cycle. All genes (see also Table 7) required are controlled
by strong methanol-inducible promotors derived from P. pastoris CBS7435
(Centraalbureau voor Schimmelcultures, NL, genome sequenced by (Küberl et al.,
2011, Valli et al. 2016). GroEL and GroES are regulated by constitutive promoters of
intermediate strength.

| Gene | | Methanol | | | | |
|---|---|---|---|---|---|---|
| Name | P_{XXX} | Induced | location ID | T_{XXX} | location ID | locus |
| PGK1 | P_{ALD4} | Yes | PP7435_chr2 | T_{AOX1} | PP7435_chr4 | AOX1 |
| | | | (1466285 . . . 1467148) | | (240891 . . . 241840 | |
| TDH3 | P_{AOX1} | Yes | PP7435_chr4 | T_{IDP1} | PP7435_chr1 | AOX1 |
| | | | (237941 . . . 238898) | | (1012481 . . . 1012975) | |
| TPI1 | P_{SHB17} | Yes | PP7435_chr2 | T_{DAS2} | PP7435_chr3 | DAS2 |
| | | | (340617 . . . 341606) | | (629173 . . . 630076) | |
| TKL1 | P_{DAS2} | Yes | PP7435_chr3 | T_{RPS2} | PP7435_chr1 | DAS2 |
| | | | (632201 . . . 633100) | | (2506918 . . . 2507385) | |
| cbbM - | P_{DAS1} | Yes | PP7435_chr3 | T_{RPS3} | PP7435_chr1 | DAS1 |
| RuBisCO | | | (634140 . . . 634688) | | (223093 . . . 223258) | |
| PRK | P_{FDH1} | Yes | PP7435_chr3 | T_{RPP1B} | PP7435_chr4 | AOX1 |
| | | | (423504 . . . 424503) | | (463560 . . . 464058) | |
| GroEL | P_{PDC1} | No | PP7435_chr3 | T_{RPS17B} | PP7435_chr2 | DAS1 |
| | | | (1860841 . . . 1861824) | | (905111 . . . 905593) | |
| GroES | P_{RPP1B} | No | PP7435_chr4 | T_{DAS1} | PP7435_chr3 | DAS1 |
| | | | (462240 . . . 463233) | | (636813 . . . 637362) | |

Within this study, three native genes of Pichia pastoris (AOX1 (ORF ID: PP7435_Chr4-0130), DAS1 (ORF ID: PP7435_Chr3-0352) and DAS2 (ORF ID: PP7435_Chr3-0350) were replaced by genes listed in Table 5 and the integration event was facilitated by a CRISPR/Cas9 mediated system relying on the DNA damage repair mechanism via homologous recombination. By providing a DNA template fragment upon introduction of a DSB, consisting of homologous regions flanking the genes which will be integrated, gene replacements can be conducted very efficiently in P. pastoris with high precision. The design of the CRISPR/Cas9 system in use was developed in accordance to (Gao et al. 2014 Journal of Integrative Plant Biology 56 (4): 343-49. doi:10.1111/jipb.12152; Weninger et al. 2016. Journal of Biotechnology 235: 139-49. doi:10.1016/j.jbiotec.2016.03.027). The construction of the plasmids in use is described in the following.

The flanking regions needed for replacing the native sequences of the enzymes Aox1, Das1 and Das2 were amplified from genomic DNA (gDNA) extracts from CBS7435 wt cells by PCR (NEB, Q5® High-Fidelity DNA Polymerase). Genomic DNA was extracted from 2 mL of an overnight culture grown in YPD medium. The gDNA was prepared according to the supplier's protocol (Promega, Wizard® Genomic DNA Purification Kit). In brief, the promoter and terminator sequences were amplified from the genome by PCR with respective primers. After amplification the sequences were checked and purified by agarose gel electrophoresis (DNA staining with SYBR® Safe or Midori Green) and respective bands were cut out and prepared according to the supplier's protocol (PROMEGA-Wizard® SV Gel and PCR Clean-Up System).

In the following, the sequences were cloned into respective backbone (BB) 1 vectors with fusion sites, which allow the combination later on with coding sequences. Golden gate plasmids were assembled in one-pot reactions. For each reaction 40 fmol of plasmid or PCR fragment which were combined was used. Reaction mixtures contained 100 U of T4 ligase (New England Biolabs Ipswich, Mass.) and 20 U of Bsal (New England Biolabs Ipswich, Mass.) (for BB1 or BB3 reactions) or Bpil (Bbsl), (ThermoFischer Scientific, US) (for BB2 reactions) in dH₂O diluted CutSmart buffer (New England Biolabs Ipswich, Mass.) supplemented with 20 mM ATP (New England Biolabs Ipswich, Mass.). Each reaction mixture was incubated in PCR tubes using a Thermocycler (37° C., 1 min and 16° C., 2.5 min for 45 repeats followed by 50° C./5 min and 80° C./10 min). The reaction mixtures were then directly used for transformation into E. coli DH10B strains. All golden gate procedure were carried out according to (Sarkari, et al. 2017 Bioresource Technology. doi:10.1016/j.biortech.2017.05.004).

A 100 μL aliquot of chemically competent cells was mixed gently with the golden gate reaction mixture and incubated on ice for 10 min followed by a heat shock at 42° C. for 90 s. After heat treatment cells were again chilled on ice for 5-10 min. After addition of 1 mL LB medium, transformed cells were regenerated at 37° C. for 30 min (for selection on kanamycin in BB1 and BB3) and for 60 min (in case of selection on ampicillin in BB2). After regeneration cells were plated in 3 different dilutions on selective LB-agar plated (20 μL, 200 μL and the remaining cells after a spin down and re-suspension in small volume of LB medium). Plates were incubated for approximately 16 h/37° C. and from there 2 mL of LB medium with respective antibiotics were inoculated with single colonies and again incubated for 12-16 h. From these cultures, mini preparations were performed according to the supplier's protocol (HiYield® Plasmid Mini Kit, SLG, Gauting, Ger) and checked by enzymatic digestion with appropriate enzymes followed by agarose gel electrophoresis and Sanger sequencing. The other CBS7435 wt derived promoters (P_{ALD4}, P_{FDH1}, P_{SHB17}, P_{PDC1} and P_{RPP1B}) and terminators (T_{IDP1}, T_{RPB1t}, T_{RPS2t}, T_{RPS3t} and T_{RPBS17Bt}) were prepared accordingly (CBS7435 wt locus IDs are listed in Table 4) and cloned into respective BB1 plasmids. Coding sequences of Tdh3 and Pgk1 were amplified from gDNA from Ogataea polymporpha (CBS 4732) and Tkl1 and Tpi1 from gDNA from Ogataea parapolymorpha (CBS 11895) according to the procedure described above. The sequences encoding the chaperones GroEL and GroES (Escherichia coli), PRK (Spinacia oleracea) and cbbM (Thiobacillus denitrificans) were codon optimized and purchased from GeneArt. After cloning of all flanking regions/promoters, coding sequences (CDS) and terminators in BB1, respective promoter-CDS-terminator fragments were combined in BB2 level (combinations shown in Table 6). Golden gate reactions and transformations were carried out as described above and integrity of plasmids was checked by restriction digestions and agarose gel electrophoresis. The last step of combining the respective expression cassettes in BB3 was carried out in modified versions of BB3 vectors with additional external Bpil sites 5′ of the first promoter and 3′ of the last terminator, which allowed the excision of the fragments after regular Bsal mediated assembly (see also column “Plasmid for linear fragment” in Table 7). The integrity of these plasmids was finally checked by restriction digestion with Bpil (Bbsl), (ThermoFischer Scientific, US) followed by agarose gel electrophoresis and partially by Sanger sequencing. Clones assigned to correct plasmid assembly were amplified and frozen in 10% glycerol cryo-stocks at −80° C. From these cryo-stocks, 100 mL flasks with LB medium were inoculated and cultivated at 37° C./250 rpm for 12-16 h. Cells were then harvested and used for plasmid midi preparations according to the supplier's protocol (HiSpeed Midi Kit, Qiagen). The entire plasmid material from the midi preparation was then digested with Bpil (Bbsl), (ThermoFischer Scientific, US) and the sample was purified in a preparative agarose gel electrophoresis. The respective bands for replacement of the three native loci were purified according to the supplier's protocol with slight modifications. All gel slices derived from the same band were dissolved in a 15 mL Falcon tube and were then loaded on to one or two columns by several repeats of the loading steps. The elution step was carried out with 50 μL and was repeated 3 times. The final solutions were again checked by gel electrophoresis before storage at −20° C.

Plasmids harboring guide RNAs (gRNAs), hCas9, an ARS/CEN sequence for episomal replication and the resistance cassette for selection of P. pastoris on G418 after transformation, were constructed using golden gate cloning as described in (Sarkari, et al. 2017 Bioresource Technology. doi:10.1016/j.biortech.2017.05.004).

The genomic recognition sites for targeting the different loci with CRSIPR/Cas9 were:

(AOX1, SEQ ID NO:9)
CTAGGATATCAAACTCTTCG,
(DAS1, SEQ ID NO: 10)
TGGAGAATAATCGAACAAAA and
(DAS2, SEQ ID NO: 11)
CGACAAACTATAAGTAGATT.

The fusion PCR was checked by agarose gel electrophoresis and respective bands were purified for further usage in golden gate assembly. The gRNA stretches were assembled into a BB3 plasmid, which allowed episomal expression (ARS/CEN) of hCas9 and the resistance cassette for G418 for selection in P. pastoris. The plasmids exhibited a linker sequence between gRNA promoter (P_{GAP}) and terminator (T_{tef1}) containing a Bpil restriction site. The purified plasmids were firstly cloned in a regular Bsal BB1 reaction and further into the hCas9 BB3 plasmid using a Bpil reaction. Correctly assembled plasmids, identified by restriction digests with appropriate enzymes, were verified by Sanger sequencing. Afterwards midi preparations were performed and DNA concentrations (both from gRNA plasmids and linear replacement fragments) were determined by NanoDrop measurements.

### Example 3 Construction of Pichia pastoris Strains Expressing a Functional Calvin Cycle Targeted to the Peroxisome

In order to create the GaT_pp_10 and the control P. pastoris strains, three genes in the P. pastoris genome were deleted, namely AOX1 (ORF ID: PP7435_Chr4-0130), DAS1 (ORF ID: PP7435_Chr3-0352) and DAS2 (ORF ID: PP7435_Chr3-0350) and eight genes were integrated PGK1, TDH3, TPI1, PRK, TKL, GroEL, GroES and RuBisCO (Table 5 and 6) into the genome.

3.1 Transformation of Pichia pastoris

P. pastoris transformations were carried out with chemically competent cells using electroporation, which is described in the following. A 10 mL YPD pre-culture was inoculated with a single colony from a P. pastoris (CBS7435 wt or respective clones) and incubated overnight (o/n; ˜16 h) (shaker; 180 rpm; 28° C.). On the next day, a 100 mL main culture was inoculated. The inoculation volume from the pre-culture was calculated as depicted in the following, so that the main culture reaches an end OD between 1.2 and 3.0 after approximately 16 h of incubation (shaker; 180 rpm; 28° C.)

$${V}_{inoc} [ µL ] = \frac{{OD}_{m} * {V}_{m}}{{e}^{μ * t}} * \frac{1000}{{OD}_{pre}}$$

ODₘ OD600 main culture after time t (use OD₆₀₀ 1.5 for calculation)

Vₘ volume main culture [mL]

tₘ incubation time of the main culture [h] (at least 15 h)

μ 0.3 h⁻¹ for P. pastoris wild type in YPD at 28° C.

ODₚᵣₑ OD₆₀₀ pre-culture

After inoculation of the main culture, OD was measured and cells were harvested in 50 mL falcon tubes by centrifugation (5 min; 1500 g and 4° C.) and re-suspended in 10 mL pre-treatment solution (0.6 M sorbitol, 10 mM Tris-HCl, 10 mM DTT, 100 mM LiCl). This mixture was incubated for 30 min (Shaker; 180 rpm; 28° C.) and filled up using ice-cold sorbitol (1 M) to 50 mL before centrifugation 5 min; 1500×g; 4° C.). Cell pellets were then combined in 45 mL of ice-cold sorbitol (1 M) and harvested by centrifugation (5 min; 1500×g; 4° C.). This washing step was repeated and then cells were re-suspended in 500 μL ice-cold sorbitol and aliquoted (80 μL) into pre-cooled Eppendorf tubes (−20° C.) on ice. The aliquots were stored at −80° C. until used in transformation.

An 80 μL aliquot of the electro-competent P. pastoris cells was mixed very gently with 1 μg of the respective gRNA-Cas9 plasmid and with 1500 to 2000 nmol of linear replacement fragment (total volume of transformation mixture did not exceed 110 μL). As a negative control, cells were transformed with an equal volume of sterile dH₂O. The mixture was then chilled on ice in 2 mm electroporation cuvettes for 5 min. Electroporation was carried out at an electroporator (2000 V, 25 μF and 186Ω). Directly after electroporation the cuvette was flushed with 1 mL YPD medium and then the entire content was transferred to Eppendorf tubes. The cells were regenerated in the Eppendorf tubes for 1.5 to 2 h at 28° C. using a thermoblock. The cells were then plated on selective YPD plates supplemented with 500 μg/mL G418 and incubated at 28° C. for 48-72 h until single colonies appeared. From these plates, single colonies were picked and restreaked twice on selective G418 plates. Positive clones were identified by colony PCR and further on restreaked on YPD plates until loss of the episomal gRNA/hCas9 plasmid occurred. This was checked by restreaking on selection plates after each restreaking passage on YPD. Positive clones derived from plasmid-free colonies were used for inoculation of 10 mL YPD and the aliquots of 1 mL were stored in the presence of 10% glycerol (v/v) at −80° C.

3.2 Verification of Transformants by Colony PCR

The integrity of the engineered loci was checked by colony PCR after two selection rounds on G418 supplemented YPD plates. For this purpose, single colonies were touched with a sterile tip and cell material was re-suspended in 10 μL NaOH (0.02 M) in PCR tubes. The tubes were incubated at 99° C. for 10 min and afterwards cooled to room temperature. From these cell lysates 3 μL were directly used as PCR templates. Appropriate primers were used for detection of the correct replacement events of AOX1, DAS1 and DAS2 loci. Loci sequences of right clones were verified by Sanger sequencing.

3.3 Engineering Workflow

TABLE 7
Strain construction overview presenting the name and parent of each
transformant with the resulting genotype starting from Pichia pastoris (Centraalbureau voor
Schimmelcultures, NL, genome sequenced by (Küberl et al., 2011, Valli et al. 2016) as wild type (wt) strain.

| | Parent | Plasmid for | | |
|---|---|---|---|---|
| Strain ID | strain ID | linear fragment | gRNA plasmid | Genotype |
| GaT_pp_04 | CBS7435 wt | GaT_B3_007 | GaT_B3_003 | Δ(aox1)₁::(TDH3, PRK, PGK1)₁ |
| | | (TDH3, PRK, PGK1) | | |
| GaT_pp_05 | CBS7435 wt | GaT_B3_008 | GaT_B3_003 | Δ(aox1)₁::(TDH3, PGK1)₁ |
| | | (TDH3, PGK1) | | |
| GaT_pp_06 | GaT_pp_04 | GaT_B3_016 | GaT_B3_012 | Δ(aox1)₁(das1)₂(das2)₃::(TDH3, |
| | | (RuBisCO, GroEL, | | PRK, PGK1)₁(RuBisCO, GroEL, |
| | | GroES) | | GroES)₂ |
| GaT_pp_07 | GaT_pp_04 | GaT_B3_017 | GaT_B3_012 | Δ(aox1)₁(das1)₂(das2)₃::(TDH3, |
| | | (RuBisCO) | | PRK, PGK1)₁(RuBisCO)₂ |
| GaT_pp_08 | GaT_pp_04 | GaT_B3_018 | GaT_B3_012 | Δ(aox1)₁(das1)₂::(TDH3, PRK, |
| | | | | PGK1)₁ |
| GaT_pp_09 | GaT_pp_05 | GaT_B3_018 | GaT_B3_012 | Δ(aox1)₁(das1)₂::(TDH3, PGK1)₁ |
| GaT_pp_10 | GaT_pp_06 | GaT_B3_027 | GaT_B3_014 | Δ(aox1)₁(das1)₂(das2)₃::(TDH3, |
| | | (TKL1, TPI1) | | PRK, PGK1)₁(RuBisCO, GroEL, |
| | | | | GroES)₂(TKL1, TPI1)₃ |
| GaT_pp_11 | GaT_pp_07 | GaT_B3_027 | GaT_B3_014 | Δ(aox1)₁(das1)₂(das2)₃::(TDH3, |
| | | (TKL1, TPI1) | | PRK, PGK1)₁(RuBisCO)₂(TKL1, |
| | | | | TPI1)₃ |
| GaT_pp_12 | GaT_pp_08 | GaT_B3_027 | GaT_B3_014 | Δ(aox1)₁(das1)₂(das2)₃::(TDH3, |
| | | (TKL1, TPI1) | | PRK, PGK1)₁(TKL1, TPI1)₃ |
| GaT_pp_13 | GaT_pp_09 | GaT_B3_027 | GaT_B3_014 | Δ(aox1)₁(das1)₂(das2)₃::(TDH3, |
| | | (TKL1, TPI1) | | PGK1)₁(TKL1, TPI1)₃ |

Strains containing all genes necessary for CO₂ assimilation are named GaT_pp_10.
GaT_pp_12 and GaT_pp_13 are control strains, which lack the key enzymes RuBisCO and PRK.

The before described procedure was applied for construction of all strains according to the workflow outlined in Table 7 (for promotor, CDS and terminator combinations see Table 5 and Table 6). The first step was the replacement of AOX1 of the P. pastoris CBS7435 wt with the expression cassette encoding for TDH3, PRK and PGK1, resulting in the strain GaT_pp_04, and for TDH3 and PGK1, delivering the strain GaT_pp_05. The integration event was facilitated by co-transformation with the gRNA/hCas9 plasmid GaT_B3_003, which creates a double strand break (DSB) at 5′ prime end of AOX1. Engineering was continued at the DAS1 locus using the gRNA/hCas9 plasmid GaT_B3_012 and co-transformation with respective linear fragments. For creation of GaT_pp_06, the DAS1 locus of GaT_pp_04 was replaced with RuBisCO, GroEL and GroES (linear fragment derived from GaT_B3_016). In the same parental strain, DAS1 was replaced with an expression cassette for RuBisCO expression without the chaperones GroEL and GroES (GaT_B3_17) and with a knock out cassette (GaT_B3_018), harboring no CDS, and the resulting strains were named GaT_pp_07 and GaT_pp_08, respectively. In the strain GaT_pp_05, DAS1 was replaced with the same knock out cassette and the transformed strained was named GaT_pp_09. In the last engineering step, the CDS of DAS1 was replaced with the expression cassette encoding for Tkl1 and Tpi1 (derived from GaT_B3_027) by co-transformation with GaT_B3_014, which created a DSB at 3′ of DAS2. The resulting strains were GaT_pp_10, GaT_pp_11, GaT_pp_12 and GaT_pp_13. The final engineered genotypes of all three strains can be obtained from Table 7 and Table 6 shows the regulatory elements used within.

### Example 4 DAS1/DAS2 Deletion Strains Containing a Functional Calvin Cycle Grow in the Presence of Carbon Dioxide and Methanol

The pre-cultures for the cultivation in bioreactors were prepared as it is described in the following.

Restreaks were made from cryo-stock solutions of GaT_pp_10, GaT_pp_12, GaT_pp_13 and CBS7435 wt on YPD-plates and incubated for 48 h on 28° C. Single colonies were picked and used for inoculation of 100 mL of YPD medium. The pre-cultures were grown over night at 28° C. and 180 rpm. Optical density was determined and cell suspension was then transferred to 50 mL Falcon tubes and centrifuged (1500 g, 6 min). The pellet was washed with sterile dH₂O twice and the resuspended in 20 mL of sterile dH₂O. From this suspension, samples were taken and OD was determined. The volume needed for inoculation of 500 mL BatchGly (starting OD=1.0 or 0.19 g*L⁻¹CDW) medium was calculated.

The bioreactor cultivations were carried out in 1.4 L DASGIP reactors (Eppendorf, Germany) with a maximum working volume of 1.0 L. Cultivation temperature was controlled at 28° C., pH was controlled at 5.0 by addition of 12.5% ammonium hydroxide and the dissolved oxygen concentration was maintained above 20% saturation by controlling the stirrer speed between 400 and 1200 rpm, and the airflow between 6 and 45 sL*h⁻¹.

The cells derived from the pre-cultures described above were used to inoculate the starting volume of 0.5 L in the bioreactor to a starting optical density (600 nm) of 1.0 or 0.19 g*L⁻¹. The glycerol batch was finished after approximately 16 h (CBS7435 wt), 36 h (GaT_pp_12, GaT_pp_13) and 40 h (GaT_pp_10). The accumulated biomass in all strains was approximately 10 g*L⁻¹CDW.

At the starting point of the fermentation a sample was taken and initial starting OD was determined in triplicates. OD measurements were carried out with a portable spectral photometer (C8000 Cell Density Meter, WPA, Biowave) in the absorbance range between 0.2 and 0.5. Sampling material from the starting point was also taken for HPLC analysis. The procedure for HPLC analysis is described in the following.

For HPLC analysis, 2 mL of cell suspension were centrifuged (13,000 rpm, 3 min) and supernatant was pipetted into a clean Eppendorf tube. Prior to a transferring the samples to glass tubes, which are suitable for the autosampling device, 900 μL supernatant were mixed with 100 μL 40 mM H₂SO4 and filtered using a 0.22 μm filter unit on a 2 mL syringe.

Glycerol, glucose, methanol and citrate were determined by HPLC as previously described using pure standards for identification and quantification (Blumhoff, et al. 2013 Metabolic Engineering 19. 26-32. doi:10.1016/j.ymben.2013.05.003). The HPLC was equipped with an Aminex HPX-87 H (300×7.8 mm, BioRad, Hercules, Calif.) column. A refraction index detector (RID-10 A, Shimadzu) was used for detection of glycerol, glucose, methanol and citrate. The column was operated at 60° C. at a flow rate of 0.6 mL/min with 0.004 M H₂SO₄ as mobile phase.

After the glycerol batch phase and throughout the cultivation, samples were taken at least once per day. HPLC samples were prepared as described above. Cell density was determined by OD measurements and by determination of cell dry mass (CDW) as described in the following.

For the determination of the cell dry weight the cell pellets from 2 mL of cell suspension were washed once with water and centrifuged (13,000 rpm, 3 min). After the washing step the cell pellets were transferred into a pre-weighted glass tube and dried for 24 h at 110° C. After drying the glass tubes are weighted again and the cell dry mass was calculated with following formula:

CDW [g/L]=(Glass tube(full) [g]−Glass tube(empty) [g])*500

For each cultivation CDW determination was done in duplicates.

After batch all bioreactors were induced by the addition of 0.5% methanol (v/v) using a 5 mL syringe connected to a 0.22 μm filter unit, which was aseptically connected to an inlet connection.

The CO₂ in the inlet gas was set to 1% during induction phase.

After the induction phase cells were pulsed with 0.5% methanol (v/v) and sampling was performed as described above. After each methanol addition, sampling was repeated for HPLC and OD analysis as described before.

The second pulse after induction was performed by adding methanol to a concentration of 0.75% (v/v) and the CO₂ concentration in the inlet air was set to 5%. Sampling was described as indicated above.

Starting with the third pulse, methanol addition was increased to 1% (v/v) once daily until the end of fermentation 1. The sampling regime was maintained as described before.

On the last day of cultivation, methanol uptake rates in the bioreactors were determined as described in the posterior section.

Cells were fed to 1% methanol and sampled as described before. Starting from there, samples were taken for HPLC measurements and OD determinations throughout the day of cultivation in approximately 1 h time spans and after 24 h.

### Results Example 4

Engineered GaT_pp_10 strains showed growth in presence of methanol as energy source and CO₂ as the sole source of carbon.

FIG. 1 shows that engineered GaT_pp_10 strains (GaT_pp_10a and GaT_pp_10b) are able to grow well in the presence of methanol as a source of energy and CO₂ as the sole carbon source. The strains lacking RuBisCO (GaT_pp_12 and GaT_pp_13) did not show significant growth after the batch end, when feeding was only done with methanol and CO₂. The disability of the RuBisCO negative strains clearly indicated that methanol cannot be incorporated into biomass anymore. It was further deduced from this experiment that the growth seen in the GaT_pp_10 strains is due to uptake and incorporation of CO₂.

RuBisCO positive GaT_pp_10 strains showed clear growth after the first pulse of methanol (see filled triangle and squares in FIG. 1) and continued growth as long as methanol was added for energy generation.

Table 8 shows the biomass formation rates observed during the entire feeding phase with methanol after the glycerol batch end. The two biological replicates of the RuBisCO positive strains, cultivated in this example, showed a biomass formation rate of 0.029 g*L⁻¹*h⁻¹ (GaT_pp_10a) and 0.016 g*L⁻¹*h⁻¹ (GaT_pp_10b) over the entire observed cultivation period. As expected, the formation of biomass under these conditions was much more pronounced in CBS7435 wt cells (0.076 g*L⁻¹*h⁻¹). CBS7435 wt cells still possess a functional DAS1 and DAS2 as well as AOX1, enabling them to assimilate and dissimilate methanol.

The control strains GaT_pp_12 and GaT_pp_13 did not show any biomass formation within the cultivation, indicating that methanol can only be utilized in the dissimilative branch of the methanol utilization pathway. This is due to a knockout of DAS1 as well as DAS2.

The biomass formation observed in the RuBisCO positive strains (GaT_pp_10a and GaT_pp_10b) is a clear indication that the synthetic assimilation pathway for CO₂ is functional.

TABLE 8
Biomass formation rate calculated over
entire co-feeding (methanol + CO₂) phase.

| | Biomass formation rate |
|---|---|
| Short Name | [gCDW * L⁻¹ * h⁻¹] |
| GaT_pp_10a | 0.029 |
| GaT_pp_10b | 0.016 |
| GaT_pp_12 | 0.000 |
| GaT_pp_13 | 0.000 |
| CBS7435 wt | 0.076 |

Formation rates are shown for all biological replicates of GaT_pp_10 (GaT_pp_10a and GaT_pp_10b), for the control strains GaT_pp_12 and GaT_pp_13 as well as for the CBS7435 wt.

In the bioreactor described in this example, methanol uptake was determined on day 6 of cultivation.

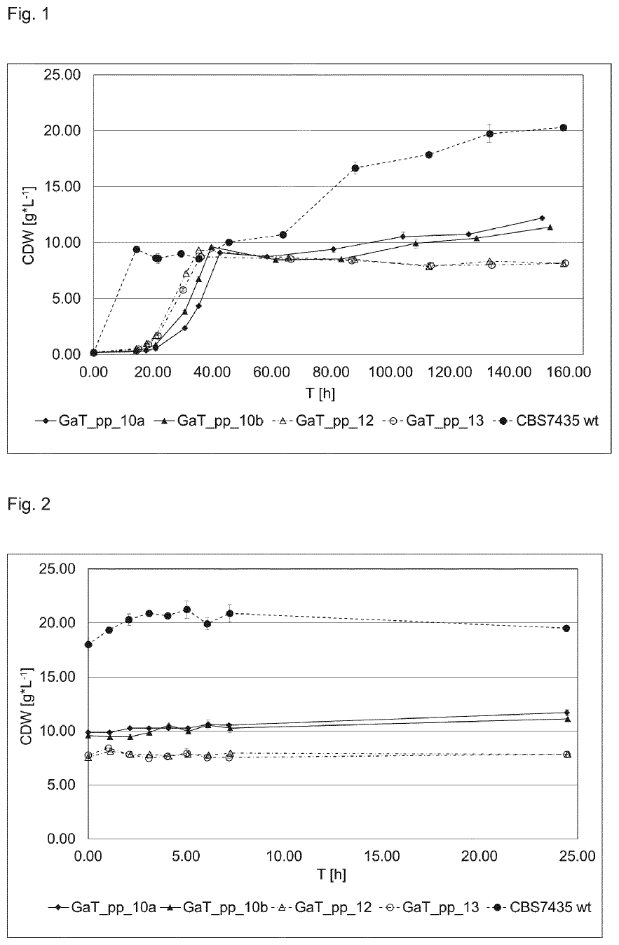

FIG. 2 shows the growth seen in all bioreactors during the study of methanol uptake.

Only engineered GaT_pp_10 strains (GaT_pp_10a and GaT_pp_10b in FIG. 2) and CBS7435 wt cells were able to grow. Growth of wt cells was expected, since these cells hold the full genetic repertoire for methanol utilization. The pronounced growth of GaT_pp_10a and GaT_pp_10b, clearly indicated the functionality of the proposed pathway for synthetic assimilation of CO₂. The introduction of the synthetic compartmentalized Calvin cycle compensates the loss of Das1 and Das2 activity and allows the strains the formation of biomass from CO₂.

The RuBisCO negative strains (GaT_pp_12 and GaT_pp_13) were not able to grow under the observed conditions. This is due to the inability to incorporate carbon, neither from methanol nor from CO₂.

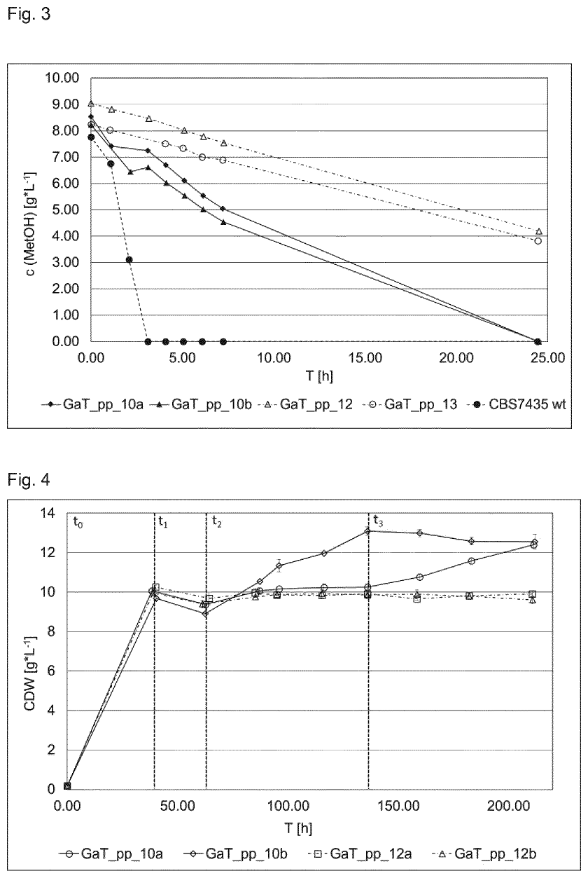

The formation of biomass in the GaT_pp_10 strains and in the CBS7435 wt also correlated with the methanol uptake observed (FIG. 3). The wt cells were able to deplete the methanol rapidly and the initial 8.0 g*L⁻¹ methanol were completely utilized within approximately 3 h. Similar to the reduced biomass formation in the GaT_pp_10 strains, the methanol uptake rate was lagging behind compared to the one observed for the wt. In the RuBisCO positive strains the initial 8.0 g*L⁻¹ methanol were reduced to ˜5 g*L⁻¹ within 7 h and completed depleted after 24 h of cultivation.

Although no growth was observed for the RuBisCO negative strains (GaT_pp_12 and GaT_pp_13), methanol was still consumed.

Table 9 shows the biomass yield on the energy source methanol Y_{X/S} and the specific methanol consumptions rates. The Y_{X/S} [g (CDW)*g (MetOH)⁻¹] value describes the gain in biomass per consumed methanol as energy equivalent in [g] CDW per [g] methanol and its calculation was only feasible for strains exhibiting growth. The biomass yield on methanol GaT_pp_10a and GaT_pp_10b is approximately half of the value observed in CBS7435 wt cells.

In order to express methanol consumption rate, the specific methanol consumption rate qₛ (MetOH) [g*g (CDW)⁻¹*h⁻¹] was calculated. In FIG. 3 the methanol uptake for the different strains are shown. The decrease in methanol concentration showed approximately linear behavior for the following time frames: for GaT_pp_10a and b, GaT_pp_12 and GaT_pp_13 until T₁˜7.2 h and for CBS7435 wt T₁˜3.1 h). The methanol consumption rate was determined from the slope of the linear regression in the aforementioned time frame. The specific methanol consumption rate (qₛ) was determined by dividing the methanol consumption rate by the biomass concentration present at T₁/2. The observation that methanol is still utilized by RuBisCO negative strains is reflected in these numbers, which show that the substrate can still be oxidized (qₛ (GaT_pp_12)=0.027, qₛ (GaT_pp_13)=0.024 [g*g⁻¹*h⁻¹]), but only with approximately 50% of the rates observed in GaT_pp_10 strains and about 25% of the rates of CBS7435 wt strain.

TABLE 9
Specific methanol consumption rate
qₛ and biomass yield on methanol Y_{X/S}.

| | Y_{X/S} | qₛ |
|---|---|---|
| Short Name | [g(CDW) * g(MetOH)⁻¹] | [g(MetOH) * g(CDW)⁻¹ * h⁻¹] |
| GaT_pp_10a | 0.213 | 0.044 |
| GaT_pp_10b | 0.186 | 0.048 |
| GaT_pp_12 | N/A | 0.027 |
| GaT_pp_13 | N/A | 0.024 |
| CBS7435 wt | 0.370 | 0.113 |

Values were determined during fermentation 1 on day 6 (example 4, FIG. 3).

### Example 5 Growth of GaT_pp_10 is Dependent on the Carbon Source CO₂ Using Methanol as Electron Donor

The YPD pre-cultures for the cultivation in bioreactors were prepared as it is described in the following.

Restreaks were made from cryo-stock solutions of GaT_pp_10, GaT_pp_11 and GaT_pp_12 on YPD-plates and incubated for 48 h on 28° C. Single colonies were picked and used for inoculation of 100 mL of YPD medium. The pre-cultures were grown over night at 28° C. and 180 rpm. Optical density was determined and cell suspension was then transferred into 50 mL Falcon tubes and centrifuged (1500 g, 6 min). The pellet was washed with sterile dH₂O twice and then resuspended in 20 mL of sterile dH₂O. From this suspension, samples were taken and OD was determined. The volume needed for inoculation of 500 mL BatchGly medium (starting OD=1.0 or 0.19 g*L⁻¹ CDW) was calculated.

The bioreactor cultivations were carried out in 1.4 L DASGIP reactors (Eppendorf, Germany) with a maximum working volume of 1.0 L. Cultivation temperature was controlled at 28° C., pH was controlled at 5.0 by addition of 12.5% ammonium hydroxide and the dissolved oxygen concentration is maintained above 20% saturation by controlling the stirrer speed between 400 and 1200 rpm, and the airflow between 6 and 45 sL*h⁻¹ during the batch phase. The inlet air was composed synthetically by a gas mixture of N₂, O₂ and CO₂ in order to ensure exact concentrations of CO₂.

The cells derived from the pre-cultures described above were used to inoculate the starting volume of 0.5 L in the bioreactor to a starting optical density (600 nm) of 1.0 or 0.19 g*L⁻¹. The glycerol batch was finished after approximately 36 h (GaT_pp_12 for both technical replicates a and b) and 40 h (GaT_pp_10 for both technical replicates a and b). The accumulated biomass in all strains was approximately 10 g*L⁻¹ CDW.

At the starting point of the fermentation 2, a sample was taken and initial starting OD was determined in triplicates. OD measurements were carried out with a portable spectral photometer (C8000 Cell Density Meter, WPA, Biowave) in the absorbance range between 0.2 and 0.5. Sampling material from the starting point was also taken for HPLC analysis. The procedure for HPLC analysis is described in example 4.

After the glycerol batch phase and throughout the cultivation, samples were taken at least once per day. HPLC samples were prepared as described above. Cell density was determined by OD measurements and by determination of cell dry mass (CDW) as described in the following.

For the determination of the cell dry weight the cell pellets from 2 mL of cell suspension were washed once with water and centrifuged (13,000 rpm, 3 min). After the washing step the cell pellets were transferred into a pre-weight glass tube and dried for 24 h at 110° C. After drying, the glass tubes are weighted again and the cell dry mass was calculated with following formula:

CDW [g/L]=(weight full glass tube [g]−weight empty glass tube [g])*500

For each cultivation CDW determination was done in triplicates.

After batch all bioreactors were induced by the addition of 0.5% methanol (v/v) using a 5 mL syringe connected to a 0.22 μm filter unit, which aseptically connected to an inlet connection.

The CO₂ in the inlet gas was set to 1% during the induction phase.

After induction of the cells under process control conditions described above, process control values of stirrer speed N and inlet gasflow rate F were increased, in order to blow out CO₂ formed by the oxidation of methanol. The stirrer speed was held constant at 1000 rpm and the gasflow rate of the inlet air mixture was set to 35 sL*h⁻¹. The CO₂ composition of the inlet gas was set to 0% for all bioreactors. This strategy was pursued to immediately blow out of all CO₂, which is inevitably formed by methanol oxidation.

After the switch to high stirring and gassing conditions the CO₂ concentration in the output flow was observed and as soon as this reached nearly 0%, methanol feeding was started.

The first feeding step after induction was done by addition of 1% methanol (v/v) in all bioreactors.

The second feeding step was done by increasing the CO₂ to 5% in the bioreactors, in which one technical replicate of GaT_pp_10b and GaT_pp_12 b respectively was cultivated.

In the other two bioreactors, in which GaT_pp_10a and GaT_pp_12a was cultivated, the CO₂ composition of the inlet air was held at 0%.

Sampling of the bioreactors was performed as described above at least once a day.

The methanol concentration was adjusted to 1% (v/v) once a day by at-line HPLC measurements.

On day 3 after induction, a switch in CO₂ was performed. The CO₂ composition of the inlet air was set from 0% to 5% for GaT_pp_10a and GaT_pp_12a. In reactors containing GaT_pp_10b and GaT_pp_12 b, CO₂ supply was changed to 0%.

After the switch on CO₂ supply, sampling and feeding carried out accordingly until the end of fermentation 2. The same procedure described above using 5% CO₂ as carbon source was conducted to test the chaperone free strain GaT_pp_11 in comparison to GaT_pp_10 (Table 10—values marked with *)

### Results Example 5

In the following section, the results of the example outline above is described and will show that the engineered GaT_pp_10 strains are able to grow on CO₂ as the sole source of carbon.

The main objective of this example was to demonstrate, that the growth in GaT_pp_10 strains is due to an external supply of CO₂ during fermentation 2. The feasibility and functionality of the proposed pathway for CO₂ assimilation was shown in example 3. Anyhow, CO₂ is also produced intracellularly by the oxidation of methanol in the first steps of the dissimilative branch of the methanol utilization pathway. In this example the process parameters were set to conditions, which ensure that produced CO₂ is immediately depleted from the cells. This was accomplished by setting the stirring rate to 1000 rpm and the gasflow rate of the gas inlet to 35 sL*h⁻¹. Under these conditions it was assured that all produced CO₂ is blown out of the bioreactor.

It was clearly visible, that directly after induction growth in engineered GaT_pp_10 strains was much more pronounced when supplied with 5% CO₂ (peaks between time point t₂ and t₃ in FIG. 4) compared to 0% CO₂ (circles between time point t₂ and t₃ in FIG. 4). This effect was also shown to be reversible, and after switching of the CO₂ supply in GaT_pp_10b (peaks after t₃ in FIG. 4), the cells stopped growing promptly. Vice versa, the cells restored growth when the CO₂ was set from 0 to 5% CO₂ GaT_pp_10a (circles after t₃ in FIG. 4)

As expected, no growth was observed in the technical replicates of the RuBisCO lacking control strain (GaT_pp_12a and b).

Biomass formation rates observed during the CO₂ supply switch fermentation 2 are summarized in Table 10 and (t) indicates that values are derived from first section of feeding phase (t₂ to t₃ in FIG. 4; i.e. 0% CO₂ in GaT_pp_10a and GaT_pp_12a; 5% CO₂ in GaT_pp_10b and GaT_pp_12 b) and (1) marked values are obtained from second phase (t₃ to end of cultivation in FIG. 4; i.e. 0% CO₂ in GaT_pp_10b and GaT_pp_12 b; 5% CO₂ in GaT_pp_10a and GaT_pp_12a).

The biomass formation values clearly indicated that formation of biomass directly correlates with the external supply of CO₂. GaT_pp_10a barely showed any (0.002 g*L⁻¹ (CDW)*h⁻¹) growth without CO₂ supply, but rapidly started to grow (0.029 g*L⁻¹ (CDW)*h⁻¹) when the CO₂ composition of the inlet air was set to 5% induction.

Vice versa, GaT_pp_10b started with well pronounced growth (0.036 g*L⁻¹ (CDW)*h⁻¹) and stopped growing (0.000 g*L⁻¹ (CDW)*h⁻¹) when CO₂ supply was set to 0%.

It was also shown in this example that growth can also be obtained by strains expressing the peroxisomal version of the synthetic Calvin cycle without co-expression of GroEL and GroES. These strains were cultivated accordingly (see values marked with * in Table 10) and the biomass formation rates observed on 5% CO₂ (0.008 g*L⁻¹ (CDW) for GaT_pp_11_a and 0.004 g*L⁻¹ (CDW) for GaT_pp_11 b) show that the pathway can work without the use of heterologous chaperones.

TABLE 10
Biomass formation rates on 0% and 5% CO₂ in the inlet gas stream.

| | Biomass formation rate 0% | Biomass formation rate 5% |
|---|---|---|
| Short Name | CO₂ [g * L⁻¹(CDW) * h⁻¹] | CO₂ [g * L⁻¹(CDW) * h⁻¹] |
| GaT_pp_10a | 0.002 ^{†} | 0.029 ^{‡} |
| GaT_pp_10b | 0.000 ^{‡} | 0.036 ^{†} |
| GaT_pp_12a | 0.000 ^{†} | 0.000 ^{‡} |
| GaT_pp_12b | 0.000 ^{‡} | 0.000 ^{†} |
| GaT_pp_10c | n/a | 0.033 * |
| GaT_pp_11a | n/a | 0.008 * |
| GaT_pp_11b | n/a | 0.004 * |

During the first phase of the fermentation (^{†}), CO₂ supply in the biological replicates GaT_pp_10a and GaT_pp_12a was 0% and was set to 5% during the second phase of the fermentation (^{‡}).
Vice versa, the first phase of the fermentation (^{†}) was conducted with 5% CO₂ in GaT_pp_10b and GaT_pp_12b II, before turning off the CO₂ during the second phase (^{‡}) in the respective bioreactors.
The growth of GaT_pp_10 strains depends on the external supply of CO₂.
In an independent replication of the fermentation phase on 5% CO₂ (*) the growth of GaT_pp_11 (a and b) was tested.

The growth data shown in this example (Table 10) demonstrate that the growth of GaT_pp_10 strains depends on the external supply of CO₂. Growth was only observable when engineered GaT_pp_10 strains, expressing a functional Calvin Cycle in the peroxisomes, were supplied with CO₂ and methanol, which demonstrates a functional uptake and incorporation of CO₂.

### Example 6 Production of Lactic Acid with Strains Expressing a Functional Synthetic Calvin Cycle Localized in the Peroxisome

The following example was conducted to demonstrate the potential of the engineered GaT_pp_10 strains as host strains for production of bulk chemicals using CO₂ as a carbon source. A broad range of pathways leading to the production of chemicals is possible using the disclosed GaT_pp_10 strains and the production of lactic acid (LA) is shown as an industrially relevant example.

P. pastoris CBS7435 variant and RuBisCO positive strains (denoted as GaT_pp_10 strains) were used as host strains. The expression vectors pPM2d_pGAP, which is a derivative of the pPuzzle_ZeoR vector backbone (described in WO2008/128701A2), and BB3rN_14 (GoldenPiCS: a Golden Gate-derived modular cloning system for applied synthetic biology in the yeast Pichia pastoris. Prielhofer R, Barrero J J, Steuer S, Gassier T, Zahrl R, Baumann K, Sauer M, Mattanovich D, Gasser B, Marx H. BMC Syst Biol. 2017 Dec. 8; 11(1):123. doi: 10.1186/s12918-017-0492-3. 10.1186/s12918-017-0492-3 PubMed 29221460) consisting of the pUC19 bacterial origin of replication and the Zeocin or a Nourseothricin (NTC) antibiotic resistance cassette. Expression of a bacterial lactate dehydrogenase (LDH) gene was mediated by the P. pastoris glyceraldehyde-3-phosphate dehydrogenase (GAP) promoter or alcohol oxidase (AOX) promoter, respectively, and the S. cerevisiae CYC1 transcription terminator. The LDH gene was sub-cloned and ligated into the vector pPM2d_pGAP and BB3rN_14, respectively, prior to electroporation into respective P. pastoris strains, as it is described in example 3. Selection of positive transformants was performed on YPD plates (per liter: 10 g yeast extract, 20 g peptone, 20 g glucose, 20 g agar-agar) containing 50 μg*mL⁻¹ of Zeocin or 100 μg*mL⁻¹ of NTC, respectively. Colony PCR was used to ensure the presence of the transformed plasmid. Therefore, genomic DNA was obtained as described in example 3 and PCR with the appropriate primers was conducted.

Finally obtained strains are denoted as GaT_pp_28 (Δ(aox1)₁(das1)₂(das2)₃::(TDH3, PRK, PGK1)₁(RuBisCO, GroEL, GroES)₂(TKL1, TPI1)₃P_{GAP}LDH) with the LDH gene under the P_{GAP} and GaT_pp_39 (Δ(aox1)₁(das1)₂(das2)₃::(TDH3, PRK, PGK1)₁(RuBisCO, GroEL, GroES)₂(TKL1, TPI1)₃P_{AOX1}LDH) with the LDH gene under the control of the AOX1 promoter (P_{AOX1}).

The LDH producing strains were then tested for LA production shake flask experiments (GaT_pp_28) and bioreactor cultivations (GaT_pp_28 and GaT_pp_39). The fermentation studies were designed according to example 4 and 5. The production of lactic acid during these cultivations was monitored by HPLC analysis (Blumhoff, et al 2013. Metabolic Engineering 19. 26-32. doi:10.1016/j.ymben.2013.05.003; Steiger, et al. 2016. Metabolic Engineering 35. 95-104. doi:10.1016/j.ymben.2016.02.003) analogous to the sample preparations described in example 3.

For shake flask cultivations strains overexpressing LDH, YP pre-cultures were prepared as follows.

Restreaks were made from cryo-stock solutions of GaT_pp_28 or GaT_pp_39 on YPD-plates and incubated for 48 h on 28° C. Single colonies were picked and used for inoculation of 100 mL of YPG medium. The pre-cultures were grown over night at 28° C. and 180 rpm. Optical density was determined and cell suspension was then transferred into 50 mL Falcon tubes and centrifuged (1500 g, 6 min). The pellet was washed with sterile dH₂O twice and then resuspended in 5 mL of sterile dH₂O. From this suspension, samples were taken and OD was determined. The volume needed for inoculation of 20 mL BatchGly medium supplemented with 0.5% methanol (starting OD=15.0 or 2.85 g*L⁻¹ CDW) was calculated.

The main cultures were then incubated in a CO₂ incubator (using 5% CO₂) on a shaking device (180 rpm). Sampling was carried out once a day after inoculation and the methanol concentration was adjusted up to 1% (v/v) from day 1 of cultivation. Cell growth (OD measurements) and metabolite profiles (HPLC analysis) were monitored as described in example 4 and 5.

For bioreactor cultivation of GaT_pp_28 or GaT_pp_39 strains, YP pre-cultures were prepared as it follows.

Restreaks were made from cryo-stock solutions of GaT_pp_28 or GaT_pp_39 on YPD-plates and incubated for 48 h on 28° C. Single colonies were picked and used for inoculation of 400 mL of YPG medium. The pre-cultures were grown over night at 28° C. and 180 rpm. Optical density was determined and cell suspension was then transferred into 500 mL sterile centrifugation tubes and centrifuged (1500 g, 6 min). The pellet was washed with sterile dH₂O twice and then resuspended in 20 mL of sterile dH₂O. From this suspension, samples were taken and OD was determined. The volume needed for inoculation of 500 mL YNB medium supplemented with 0.5% methanol (starting OD=15.0 or 2.85 g*L⁻¹ CDW) was calculated.

After inoculation, the bioreactor cultivations were carried out in 1.4 L DASGIP reactors (Eppendorf, Germany) as described for example 4 with the alteration that the pH was adjusted using 5 M NaOH. The sampling procedure and maintenance of the methanol concentration in the reactors was also performed according to example 4.

### Results Example 6

Three biological replicates were cultivated with two technical replicates each. The shake flask cultivations were maintained under an elevated CO₂ atmosphere of 5% after inoculation. During the cultivation time, the engineered strained containing LDH secreted lactic acid (LA) (Table 11).

TABLE 11
Lactic acid titers measured during cultivation of GaT_pp_28 on CO2
in shake flasks. LA titers are shown for two technical replicates (I and II) for each
biological replicate (GaT_pp_28_C1-C3) and for the parent strain (GaT_pp_10) at
different time points, at time point 0 the cells were inoculated in BatchGly medium
containing 0.5% methanol (v/v). Engineered GaT_pp_28 strains produce lactic acid
(LA) on CO₂ as a carbon source.

| | GaT_pp_10 | | GaT_pp_28_C1 | | GaT_pp_28_C2 | | GaT_pp_28_C3 | |
|---|---|---|---|---|---|---|---|---|
| | [mg/L] | | [mg/L] | | [mg/L] | | [mg/L] | |
| Time [h] | I | II | I | II | I | II | I | II |
| 0 | 0.0 | 0.0 | 0.0 | 0.0 | 0.0 | 0.0 | 0.0 | 0.0 |
| 24 | 0.0 | 0.0 | 33.5 | 0.0 | 35.0 | 34.5 | 34.9 | 34.8 |
| 48.5 | 0.0 | 0.0 | 33.7 | 33.8 | 34.1 | 34.4 | 36.3 | 35.7 |

GaT_pp_28 cells produce Lactic acid with titers up to 36 mg/L during cultivation on CO₂ as sole carbon source. These results show that the engineered yeast cells equipped with a synthetic Calvin cycle localized in the peroxisomes can be used as production platform for LA.

TABLE 12
Lactic acid titers measured during cultivation of
GaT_pp_28 and GaT_pp_39 on CO2 in a bioreactor.

| | GaT_pp_39 | | GaT_pp_28 | |
|---|---|---|---|---|
| Time [h] | CDW [g/L] | LA [mg/L] | CDW [g/L] | LA [mg/L] |
| 0 | 2.36 | 0.0 | 2.43 | 47.5 |
| 18 | 2.30 | 40.1 | 2.43 | 125.3 |

Engineered GaT_pp_39 and GaT_pp_28 strains produce lactic acid (LA) using CO2 as the sole carbon source; LA titers are shown for different time points with the corresponding cell dry weight (CDW) values.

Within the course of example 6, the engineered yeast cells harboring the peroxisomal version of the synthetic Calvin cycle were tested for LA production under the control of two different promoters. In the strain GaT_pp_39 the LDH gene is controlled by P_{AOX1} while in the GaT_pp_28 strain under the control of P_{GAP}. In both strains detectable levels of LA were obtained during bioreactor cultivations (see Table 12).

With example 6, evidence is provided that the engineered cells expressing a peroxisomal version of the synthetic Calvin cycle can be used as production platform for LA. This illustrates the possibility of the GaT_pp_10 strains as a production platform for a wide range of chemicals.

### Example 7 Production of Porcine Carboxypeptidase B (CpB) or Human Serum Albumin (HSA) with Strains Expressing a Functional Synthetic Calvin Cycle Localized in the Peroxisome

Based on the strain having a peroxisomal version of the Calvin cycle (GaT_pp_10), strains were engineered overexpressing CpB (GaT_pp_31) and (GaT_pp_35) The CpB and HSA expressing transformants were cultivated in bioreactors using CO₂ as sole carbon source. The set-up of these studies is designed accordingly to the set-ups described in example 4 and 5.

Construction of Strains

P. pastoris CBS7435 variant and RuBisCO positive strains (denoted as GaT_pp_10 strains) were used as host strains. The pPM2d_pAOX expression vector is a derivative of the pPuzzle ZeoR vector backbone described in WO2008/128701A2, consisting of the pUC19 bacterial origin of replication and the Zeocin antibiotic resistance cassette. Expression of the heterologous genes was mediated by the P. pastoris alcohol oxidase (AOX1) promoter (P_{AOX1}), respectively, and the S. cerevisiae CYC1 transcription terminator. The gene encoding porcine carboxypeptidase (amino acids 16-416 of GeneBank CAB46991.1 with 45.7 kDa) was codon optimized for P. pastoris and synthesized with the N-terminal S. cerevisiae alpha mating factor signal leader sequence. The gene encoding human serum albumin with its native secretion leader (amino acids 1-609 of GenBank NP_000468 with 66.4 kDa) was codon optimized for P. pastoris and synthesized. The molecular masses have been calculated using the Expasy online tool (https://web.expasy.org/compute_pi/). The obtained vectors carrying the genes of interests with an N-terminal secretion leader sequence were digested with SbfI and SfiI and the genes are each ligated into the vector pPM2d_pAOX digested with SbfI and SfiI. Plasmids were linearized prior to electroporation into respective P. pastoris strains (GaT_pp_10), as it was described in example 3. Selection of positive transformants was performed on YPD plates (per liter: 10 g yeast extract, 20 g peptone, 20 g glucose, 20 g agar-agar) containing 50 μg*mL⁻¹ of Zeocin. Colony PCR was used to ensure the presence of the transformed plasmid. Therefore, genomic DNA was obtained as described in example 3 and PCR with the appropriate primers was conducted.

Finally engineered strains (have the genotype Δ(aox1)₁(das1)₂(das2)₃::(TDH3, PRK, PGK1)₁(RuBisCO, GroEL, GroES)₂(TKL1, TPI1)₃P_{AOX1}CpB denoted as GaT_pp_31 and Δ(aox1)₁(das1)₂(das2)₃::(TDH3, PRK, PGK1)₁(RuBisCO, GroEL, GroES)₂(TKL1, TPI1)₃P_{AOX1}HSA denoted as GaT_pp_35. In both strains the model protein (CpB in GaT_pp_31−; HSA in GaT_pp_35) is controlled by P_{AOX1}.

For bioreactor cultivation of GaT_pp_31 and GaT_pp_35 pre-cultures were prepared as follows:

Restreaks were made from cryo-stock solutions of GaT_pp_31 and GaT_pp_35 on YPD-plates and incubated for 48 h on 28° C. Single colonies were picked and used for inoculation of 400 mL of YPG medium. The pre-cultures were grown over night at 28° C. and 180 rpm. Optical density was determined and cell suspension was then transferred into 500 mL sterile centrifugation tubes and centrifuged (1500 g, 6 min). The pellet was washed with sterile dH₂O twice and then resuspended in 20 mL of sterile dH₂O. From this suspension, samples were taken and OD was determined. The volume needed for inoculation of 500 mL YNB medium supplemented with 0.5% methanol (starting OD=18.0 or 3.45 g*L⁻¹ CDW) was calculated.

After inoculation, the bioreactor cultivations were carried out in 1.4 L DASGIP reactors (Eppendorf, Germany) as described for example 4 with the alteration that the pH was adjusted using 5 M NaOH. The sampling procedure and maintenance of the methanol concentration in the reactors was also performed according to example 4.

HSA and CpB in the culture supernatant samples was detected by SDS-PAGE analysis followed by silver ion staining. In brief, 15 μL of supernatant were mixed with 5 μL 4× sample buffer (NuPAGE LDS Sample Buffer (4×) (ThermoFischer Scientific, US)) and heated for 10 min at 70° C. before loading onto 10% Bis-Tris Protein Gels (ThermoFischer Scientific, US) in MOPS running buffer. Separation was conducted by setting the power supply to constant current at 30 mA. The gels were ran for approximately 3 h and then fixed over night at 4° C. in fixing solution (ethanol 50% (v/v), acetic acid 10% (v/v). After the fixing step, the gels were incubated for 30 min at room temperature in incubation solution (ethanol 30% (v/v), 0.89 M sodium acetate, 13 mM sodium thiosulfate, 0.25% glutaraldehyde) and washed for 3 times in RO—H₂O for 10 min each. Afterwards the gels were incubated in silver nitrate solution (6 mM silver nitrate, 0.02% formaldehyde), briefly washed and then developed in developing solution (0.25 M sodium carbonate, 0.01% formaldehyde) until bands appeared. The reaction was stopped by applying 50 mM sodium EDTA solution for 1 h.

### Results Example 7

The strain GaT_pp_31 was cultivated in bioreactor cultivation as described above and the cultivation was carried in YNB medium supplemented with 0.5% methanol. Starting from day 1, the methanol was adjusted to 1% methanol (v/v) once daily and CO₂ was supplied in the inlet gasflow (5%) representing the only carbon source for the engineered cells. During the cultivation time the cells grew with a biomass formation rate of 0.019 g CDW L⁻¹ h⁻¹. Furthermore, the analysis by SDS-PAGE and silver ion staining of supernatant samples revealed the expression of CpB by GaT_pp_31 strains (FIG. 7). After inoculation (0 hours) of the bioreactor cultivation no band was visible (lane 1 in FIG. 7), while a band can be detected at the corresponding size of approximately 45 kDa after 72 hours (lane 2 in FIG. 7). This shows that CpB is produced under conditions in which only CO₂ is available as a carbon source.

The strain overexpressing the HSA (GaT_pp_35) was cultivated accordingly to strain GaT_pp_31. In the second fermentation (biomass formation rate was 0.013 g CDW L⁻¹h⁻¹) HSA was produced in detectable levels highlighting the reproducibility of this procedure. FIG. 8 shows that HSA is accumulated during the course of the bioreactor cultivation starting from undetectable levels on day 0 (d0) to detectable levels on day 1 to day 3 (d1-3). Due to the compact and globular form of HSA the apparent molecular mass detected by silver staining (here around 55 kDa) is smaller than the actual molecular mass of 66.4 kDa (congruent with unpublished previous data).

With this example the usability of cells equipped with a peroxisomal version of the synthetic Calvin cycle as a protein production platform is demonstrated. The expression of CpB, as a model technical enzyme, and HSA, as a model protein for pharmaceutical relevant products, in well detectable levels underpins that various product classes can be produced in the RuBisCO positive (GaT_pp_10) background.

### Example 8 Impact of Aox1, Das1 and Das2 on P. pastoris Strains Expressing a Functional Calvin Cycle

The CDS of AOX1 is reintegrated into GaT_pp_10 as follows:

The CDS of AOX1 is amplified from the genome of P. pastoris CBS7435 and is cloned into respective BB1 plasmids accordingly to the procedure described in example 2. An expression cassette harboring the native P_{AOX1}, the CDS of AOX1 and a suitable terminator is constructed in BB2 level by Golden Gate cloning as outlined in example 2. A functional AOX1 cassette is integrated into the GaT_pp_10 strain using a similar workflow as describe in example 2 and 3 using Golden Gate cloning and CRISPR/Cas9.

Similar to the workflow described above for the reconstitution of Aox1 activity, the CDSs of DAS1 and DAS2 are reintegrated into the respective terminator regions of the engineered strains.

Strains are tested for growth on CO₂ and methanol as described in example 4, 5 and 6.

### Example 9 ¹³C Labelling to Verify CO₂ Incorporation in P. pastoris Strains Expressing a Functional Calvin Cycle

¹³C based labelling studies were conducted to analyze the incorporation of inorganic carbon via uptake of gaseous CO₂ into the biomass. The experimental set-up (adapted according to example 4) involves a batch phase on ¹³C labelled glycerol followed by a feeding phase on labelled ¹²C CO₂ and un-labelled ¹³C methanol (scenario I). In a second setup the batch is also carried out with ¹³C labelled glycerol and the feeding phase is done with ¹²C un-labelled methanol and un-labelled (¹²C) CO₂ (scenario II).

The cultivations are performed in bioreactors according to the procedures described in example 5 with the strains GaT_pp_10 and GaT_pp_12. Contrary to example 5, Labelling Medium (LM) was used containing fully labelled ¹³C glycerol as a carbon source. In total, four bioreactors were inoculated. In three bioreactors scenario I was applied (two times GaT_pp_10 and one reactor with GaT_pp_12), while scenario II was applied to a reactor inoculated with GaT_pp_10. From both experiments the biomass was harvested and the isotope ratio in the biomass of ¹²C to ¹³C is determined by using an elemental analyzer coupled to an Isotope Ratio Mass Spectrometer (EA-IRMS). This analytical procedure was carried out in as a commercial service by a third party (IMPRINT ANALYTICS, Neutal, Austria).

In brief, restreaks were made from cryo-stock solutions of GaT_pp_10 and GaT_pp_12 on YPD-plates and incubated for 48 h on 28° C. Single colonies were picked and used for inoculation of 100 mL of YPD medium. The pre-cultures were grown over night at 28° C. and 180 rpm. Optical density was determined and cell suspension was then transferred into 50 mL Falcon tubes and centrifuged (1500 g, 6 min). The pellet was washed with sterile dH₂O twice and then resuspended in 20 mL of sterile dH₂O. From this suspension, samples were taken and OD was determined. The volume needed for inoculation of 500 mL LM (starting OD=1.0 or 0.19 g*L⁻¹ CDW) was calculated.

The bioreactor cultivations were carried out in 1.4 L DASGIP reactors (Eppendorf, Germany) with a maximum working volume of 1.0 L. Cultivation temperature was controlled at 28° C., pH was controlled at 5.0 by addition of 12.5% ammonium hydroxide and the dissolved oxygen concentration is maintained above 20% saturation by controlling the stirrer speed between 400 and 1200 rpm, and the airflow between 6 and 45 sL*h⁻¹ during the batch phase. The inlet air was composed synthetically by a gas mixture of N₂, O₂ and CO₂ in order to ensure exact concentrations of CO₂.

The cells derived from the pre-cultures described above were used to inoculate the starting volume of 0.5 L in the bioreactor to a starting optical density (600 nm) of 1.0 or 0.19 g*L⁻¹. The glycerol batch was finished after approximately 36 h (GaT_pp_12) and 40 h (GaT_pp_10 for all three technical replicates I to III). The accumulated biomass in all strains was approximately 5.0 g*L⁻¹ CDW.

At the starting point of the labelling fermentation, a sample was taken and initial starting OD was determined in triplicates. OD measurements were carried out with a portable spectral photometer (C8000 Cell Density Meter, WPA, Biowave) in the absorbance range between 0.2 and 0.5. Sampling material from the starting point was also taken for HPLC analysis. The procedure for HPLC analysis is described in example 4. Additionally, samples were taken for determination of total 130 content by EA-IRMS. To this end, a volume of cell suspension corresponding approximately 0.5 mg of dried biomass was firstly washed with 0.1 M HCL and then twice with RO—H₂O. Until the analysis, the ¹³C biomass samples were stored at −20° C.

After the glycerol batch phase and throughout the cultivation, samples were taken at least once per day. OD measurements, HPLC and ¹³C content sample preparations were done as described above.

After batch all bioreactors were induced by the addition of 0.5% methanol (v/v) using a 5 mL syringe connected to a 0.22 μm filter unit, which aseptically connected to an inlet connection.

The CO₂ in the inlet gas was set to 1% during the induction phase.

After induction of the cells under process control conditions described above, process control values of stirrer speed N and inlet gasflow rate F were increased, in order to blow out CO₂ formed by the oxidation of methanol. The stirrer speed was held constant at 1000 rpm and the gasflow rate of the inlet air mixture was set to 35 sL*h⁻¹¹.

After induction, the feeding phase was started by increasing the CO₂ to 5% and by addition of 1% methanol (v/v) in all bioreactors

Sampling of the bioreactors was performed as described above at least once a day.

The methanol concentration was adjusted to 1% (v/v) once a day by at-line HPLC measurements. In the reactors with the control strain GaT_pp_12 and two reactor containing the strain GaT_pp_10 (I and II), ¹³C labelled methanol was applied (scenario I) while in the third reactor with the strain GaT_pp_10 (III) un-labelled ¹²C methanol was used (scenario II).

### Results Example 9

In the following section, the results of the example outlined above is described and will show that the engineered GaT_pp_10 strains are able to grow on CO₂ as the sole source of carbon and that formation of biomass is due to uptake of gaseous CO₂

The growth performance during the labelling experiment on Labelling medium using CO₂ as the sole carbon source and methanol as a donor substrate for the generation of reduction equivalents was similar to examples 4 and 5 where BatchGly medium was used. This is reflected in similar biomass formation rates during the growth on CO₂ and methanol (compare Table 10 and 13). Further, utilization of ¹³C labelled methanol (GaT_pp_I and II) or un-labelled methanol (III) does not change growth performance significantly.

TABLE 13
Biomass formation rates during ¹³C labelling
fermentation of strains GaT_pp_10 and GaT_pp_12.

| | | Biomass formation rate |
|---|---|---|
| Short Name | Strain | [gCDW * L⁻¹ * h⁻¹] |
| GaT_pp_12 | GaT_pp_12 | 0.000 |
| GaT_pp_10 I | GaT_pp_10 | 0.041 |
| GaT_pp_10 II | GaT_pp_10 | 0.036 |
| GaT_pp_10 III | GaT_pp_10 | 0.042 |

Either cultivated on ¹³C methanol in the presence of ¹²C CO2 (GaT_pp_12 and GaT_pp_10 I-II) or on ¹²C methanol (GaT_pp_10 III) in the presence of ¹²C CO₂ (after a batch phase on ¹³C glycerol).

TABLE 14
Total ¹³C content analysis of biomass samples by Isotope Ratio Mass
Spectrometry (EA-IRMS) of strains GaT_pp_12 and GaT_pp_10. All
strains were grown on ¹³C glycerol (Batch) followed by a co-feed on
¹²CO₂/¹³CH₃OH (scenario I-GaT_pp_12, GaT pp_10 I-II) or on
¹²CO₂/¹²CH₃OH (scenario II-GaT_pp_10 III). Measured ¹³C content
in % (¹³C^{m}) in biomass samples obtained by EA-IRMS. Standard
deviation of ¹³C^{m} shows the error of three technical replicated
measurements of the same sample. Expected, theoretic ¹³C content
in % (¹³C^{cal}) calculated using the measured biomass formation.

| | GaT_pp_12 | | GaT_pp_10 I | | GaT_pp_10 II | | GaT_pp_10 III | |
|---|---|---|---|---|---|---|---|---|
| | Strain | | | | | | | |
| | GaT_pp_12 | | GaT_pp_10 | | GaT_pp_10 | | GaT_pp_10 | |
| | C-source | | | | | | | |
| | ¹²CO₂/ | | ¹²CO₂/ | | ¹²CO₂/ | | ¹²CO₂/ | |
| | ¹³CH₃OH | | ¹³CH₃OH | | ¹³CH₃OH | | ¹³CH₃OH | |
| Time [h] | ¹³C^{cal} | ¹³C^{m} | ¹³C^{cal} | ¹³C^{m} | ¹³C^{cal} | ¹³C^{m} | ¹³C^{cal} | ¹³C^{m} |
| 45 (Batch | 95% | 95 ± 0.5% | 95% | 97 ± 0.1% | 95% | 97 ± 0.3% | 95% | 97 ± 0.1% |
| end) | | | | | | | | |
| 85 | 95% | 95 ± 0.8% | 79% | 76 ± 0.9% | 75% | 77 ± 0.6% | 67% | 72 ± 0.2% |
| 133 | 95% | 95 ± 0.3% | 55% | 57 ± 0.6% | 54% | 58 ± 0.1% | 50% | 50 ± 0.4% |
| 158 | 95% | 96 ± 0.5% | 48% | 52 ± 0.3% | 47% | 48 ± 0.2% | 42% | 43 ± 0.4% |

Example 9 verifies the incorporation of CO₂ into the biomass directly by measuring the total ¹³C content by EA-IRMS upon growth on ¹²CO₂. The ¹³C content in the biomass was enriched during the batch phase on ¹³C glycerol to 95% (see Batch end values at 45 hours in Table 14) and then washed out by applying ¹²CO₂ as a carbon source. The strain GaT_pp_12 is a control strain, which contains no functional Calvin cycle, and consequently is not able to change its ¹³C content by incorporating ¹²C CO₂. All growing strains (GaT_pp_10 I-III) showed a reduction in ¹³C content during the co-feeding phase (see ¹³C^{m} values after 85-158 hours in Table 14) which was comparable to values calculated according to the accumulated biomass (¹³C cal values at respective time points). For the two strains fed with ¹³C methanol for energy supply (GaT_pp_10 I and II), the ¹³C content was reduced according to the theoretical value. This shows that no significant amounts of carbon stemming from the methanol oxidation itself are incorporated. In scenario II (GaT_pp_10 III) ¹²C methanol was used for energy supply. In this approach the degree of total ¹³C content reduction in the final biomass is not significantly different from scenario I (GaT_pp_10 I and II). This shows that the assimilated carbon comes from the ¹²CO₂ supplied in the inlet gasflow and not from methanol oxidation itself.

### Example 10: Plasmid and Strain Construction for Cytosolic Expression of a Calvin Cycle in P. pastoris

In this example, the construction of a strain is disclosed, which contains a functional Calvin cycle localized to the cytosol. All steps to amply and subclone DNA into plasmids using Golden Gate cloning are carried out as described in Example 2. The coding sequences (CDS) of the genes mentioned in Table 15 were combined with methanol inducible promoters and terminator sequences from Pichia pastoris CBS7435 wt (Table 16).

TABLE 15
Genes required for the creation of the synthetic Calvin cycle localized
to the cytsol in Pichia pastoris with gene source, according enzymatic nomenclature
and EC number.

| | SEQ | | | | | PTS |
|---|---|---|---|---|---|---|
| Gene Name | ID NO | UniProt* | Source | EC Number | Full Name | added |
| cRuBisCO | 37 | Q60028 | Thiobacillus | 4.1.1.39 | Ribulose-bisphosphate | NO |
| | | | denitrificans (ATCC | | carboxylase | |
| | | | 25259) | | | |
| cPRK | 38 | P09559.1 | Spinacia oleracea | 2.7.1.19 | Phosphoribulokinase | NO |
| cPGK1 | 39 | A0A1B7SCV2 | Ogataea polymporpha | 2.7.2.3 | Phosphoglycerate kinase | NO |
| | | | (CBS 4732) | | | |
| cTDH3 | 40 | A0A1B7SCG5 | Ogataea polymporpha | 1.2.1.12 | Glyceraldehyde-3- | NO |
| | | | (CBS 4732) | | phosphate | |
| | | | | | dehydrogenase | |
| cTPI1 | 41 | W1Q838 | Ogataea | 5.3.1.1 | Triosephosphate | NO |
| | | | parapolymorpha | | isomerase | |
| | | | (CBS11895) | | | |
| cTKL1 | 42 | W1QKQ2 | Ogataea | 2.2.1.1 | Transketolase | NO |
| | | | parapolymorpha | | | |
| | | | (CBS11895) | | | |
| GroEL | 43 | B1XDP7 | Escherichia coli | N/A | molecular chaperone | NO |
| | | | (DH10B) | | GroEL | |
| GroES | 8 | B1XDP6 | Escherichia coli | N/A | molecular chaperone | NO |
| | | | (DH10B) | | GroES | |

TABLE 16
Gene regulation elements (promoters P_{XXX} and terminators T_{XXX}) in
proposed synthetic Calvin cycle. All genes (see also Table 9) are controlled by strong
methanol-inducible promotors derived from P. pastoris CBS 7435. GroEL and GroES
are regulated by constitutive promoters of intermediate strength.

| Gene | | Methanol | | | | |
|---|---|---|---|---|---|---|
| Name | P_{XXX} | Induced | location ID | T_{XXX} | location ID | locus |
| cPGK1 | P_{ALD4} | Yes | cbs7435_chr2 | T_{AOX1} | cbs7435_chr4 | AOX1 |
| | | | (1466285 . . . 1467148) | | (240891 . . . 241840 | |
| cTDH3 | P_{AOX1} | Yes | cbs7435_chr4 | T_{IDP1} | cbs7435 chr1 | AOX1 |
| | | | (237941 . . . 238898) | | (1012481 . . . 1012975) | |
| cTPI1 | P_{SHB17} | Yes | cbs7435_chr2 | T_{DAS2} | cbs7435_chr3 | DAS2 |
| | | | (340617 . . . 341606) | | (629173 . . . 630076) | |
| cTKL1 | P_{DAS2} | Yes | cbs7435_chr3 | T_{RPS2} | cbs7435 chr1 | DAS2 |
| | | | (632201 . . . 633100) | | (2506918 . . . 2507385) | |
| cRuBisCO | P_{DAS1} | Yes | cbs7435_chr3 | T_{RPS3} | cbs7435 chr1 | DAS1 |
| | | | (634140 . . . 634688) | | (223093 . . . 223258) | |
| cPRK | P_{FDH1} | Yes | cbs7435_chr3 | T_{RPP1B} | cbs7435 chr4 | AOX1 |
| | | | (423504 . . . 424503) | | (463560 . . . 464058) | |
| GroEL | P_{PDC1} | No | cbs7435_chr3 | T_{RPS17B} | cbs7435_chr2 | DAS1 |
| | | | (1860841 . . . 1861824) | | (905111 . . . 905593) | |
| GroES | P_{RPP1B} | No | cbs7435_chr4 | T_{DAS1} | cbs7435_chr3 | DAS1 |
| | | | (462240 . . . 463233) | | (636813 . . . 637362) | |

The expression cassettes listed in Table 16 were assembled with Golden Gate cloning and used for transformation of P. pastoris CBS7435 according to the procedure described in Example 3.

Strain GaT_pp_22 was constructed according to the scheme presented in Table 17. This strain contains all necessary genes to enable a cytosolic Calvin cycle in P. pastoris.

TABLE 17
Strain construction overview presenting the
name and parent of each transformant with the resulting genotype starting from Pichia
pastoris (Centraalbureau voor Schimmelcultures, NL, genome sequenced by (Küberl et al., 2011; Valli et al., 2016).

| | Parent | Plasmid for | | |
|---|---|---|---|---|
| Strain ID | strain ID | linear fragment | gRNA plasmid | Genotype |
| GaT_pp_16 | CBS7435 wt | GaT_B3_038 | GaT_B3_040 | Δ(aox1)₁::(cTDH3, cPRK, cPGK1)₁ |
| | | (cTDH3, cPRK, cPGK1) | | |
| GaT_pp_18 | GaT_pp_16 | GaT_B3_045 | GaT_B3_030 | Δ(aox1)₁(das2)₂::(cTDH3, cPRK, |
| | | (cTKL1, cTPI1) | | cPGK1)₁ (cTKL1, cTPI1)₂ |
| GaT_pp_22 | GaT_pp_18 | GaT_B3_043 | GaT_B3_012 | Δ(aox1)₁(das2)₂(das1)₃::(cTDH3, |
| | | (cRuBisCO, GroEL, GroES) | | cPRK, cPGK1)₁ (cTKL1, |
| | | | | cTPI1)₂(cRuBisCO, GroEL, GroES)₃ |

Strains containing all genes necessary for CO₂ assimilation with a cytosolic version of the Calvin cycle are named GaT_pp_22.

### Example 11 A Strain Containing a Functional Calvin Cycle Localized to the Cytosol can Grow in the Presence of Carbon Dioxide and Methanol

Bioreactor cultivations were carried out as described in Example 4. The batch phase was carried out with 15 g/L glycerol. Feeding with CO₂ and methanol was carried out as described in Example 4.

Engineered GaT_pp_22 strains showed growth in presence of methanol as energy source and CO₂ as the sole source of carbon during the methanol/CO₂ feeding phase.

FIG. 6: shows that engineered GaT_pp_22 strains are able to grow in the presence of methanol, as a source of energy, and CO₂, as the sole carbon source. From this experiment, it can be concluded that the growth seen in the GaT_pp_22 strains is due to uptake and incorporation of CO₂. Table 18 shows the biomass formation rates observed during the entire feeding phase with methanol after the glycerol batch end of strain GaT_pp_22 (I and II) compared to the strain having a pathway localized to the peroxisome (GaT_pp_10 I and II).

The biomass formation observed in the RuBisCO positive strains (GaT_pp_22 I and GaT_pp_22 II) demonstrates that the synthetic assimilation pathway for CO₂ is functional (Table 18).

TABLE 18
Biomass formation rate calculated over
entire co-feeding (methanol + CO₂) phase.

| | Biomass formation rate |
|---|---|
| Short Name | [gCDW * L⁻¹ * h⁻¹] |
| GaT_pp_10 I | 0.038 |
| GaT_pp_10 II | 0.039 |
| GaT_pp_22 I | 0.032 |
| GaT_pp_22 II | 0.034 |

Formation rates are shown for two biological replicates of GaT_pp_22 (I and II) and compared to two biological replicates of GaT_pp_10 (I and II) expressing a cytosolic pathway version.

### Example 12 Production of Lactic Acid with Strains Expressing a Functional Synthetic Calvin Cycle Localized in the Cytosol (GaT_pp_22)

The following example was conducted to demonstrate the potential of the engineered GaT_pp_22 strains as host strains for production of bulk chemicals using CO₂ as a carbon source. A broad range of pathways leading to the production of chemicals is possible using the disclosed GaT_pp_22 strains and the production of lactic acid (LA) is shown as an industrially relevant example.

The plasmid constructed in Example 6 containing LDH under the control of P_{AOX1} was transformed into strain GaT_pp_22 yielding GaT_pp_41 with the full genotype: Δ(aox1)₁(das1)₂(das2)₃::(cTDH3, cPRK, cPGK1)₁(cRuBisCO, GroEL, GroES)₂(cTKL1, cTPI1)₃P_{AOX1}LDH.

The LDH producing strains were then tested for lactic acid (LA) production in fermentation studies, which are designed according to Examples 4, 5 and 6. The production of lactic acid during these cultivations was monitored by HPLC analysis (Blumhoff, et al 2013. Metabolic Engineering 19. 26-32. doi:10.1016/j.ymben.2013.05.003; Steiger, et al. 2016. Metabolic Engineering 35. 95-104. doi:10.1016/j.ymben.2016.02.003) analogous to the sample preparations described in Example 3.

In brief, bioreactor cultivation of GaT_pp_41 strains overexpressing LDH were performed as it follows.

Restreaks were made from cryo-stock solutions of GaT_pp_41 on YPD-plates and incubated for 48 h on 28° C. Single colonies were picked and used for inoculation of 400 mL of YPG medium. The pre-cultures were grown over night at 28° C. and 180 rpm. Optical density was determined and cell suspension was then transferred into 500 mL sterile centrifugation tubes and centrifuged (1500 g, 6 min). The pellet was washed with sterile dH₂O twice and then resuspended in 20 mL of sterile dH₂O. From this suspension, samples were taken and OD was determined. The volume needed for inoculation of 500 mL YNB medium supplemented with 0.5% methanol (starting OD=15.0 or 2.85 g*L⁻¹ CDW) was calculated.

After inoculation, the bioreactor cultivations were carried out in 1.4 L DASGIP reactors (Eppendorf, Germany) as described for example 4 with the alteration that the pH was adjusted using 5 M NaOH. The sampling procedure and maintenance of the methanol concentration in the reactors was also performed according to example 4.

### Results Example 12

In Example 6, it was shown that the GaT_pp_10 strains (peroxisomal version of the pathway) can be used as a production platform of LA. In this example 12, data is provided showing that also strains expressing the synthetic Calvin cycle in the cytosol can be used for LA production.

In the bioreactor cultivation the engineered GaT_pp_41 cells were able to grow and secrete lactic acid in the supernatant (Table 19). Up to 35 mg/L lactic acid was detected after 42 hours of cultivation.

TABLE 19

| | GaT_pp_41 | |
|---|---|---|
| Time [h] | CDW [g/L] | LA [mg/L] |
| 0 | 2.36 | 0.0 |
| 18 | 2.11 | 0.0 |
| 42 | 3.83 | 34.8 |

The engineered GaT_pp_41 strain produce lactic acid (LA) using CO2 as the sole carbon source; LA titer is shown for different time points with the corresponding cell dry weight (CDW) values

The data provided here demonstrates the possibility to accumulate lactic acid using CO₂ as the sole source of carbon, while energy is provided by methanol oxidation in the background of GaT_pp_22.

### Example 13 Production of Itaconic Acid with Strains Expressing a Functional Synthetic Calvin Cycle Localized in the Cytosol (GaT_pp_22) and the Peroxisome (GaT_pp_10)

The following example is conducted to demonstrate the potential of further engineered GaT_pp_22 and GaT_pp_10 strains as host strains for the production of itaconic acid using CO₂ as a carbon source.

The previously described strains GaT_pp_22 and GaT_pp_10 are used as recipient strains and are transformed with a plasmid containing a functional expression cassette transcribing the coding sequence of cadA encoding a cis-aconitate decarboxylase (Uniprot ID: B3IUN8). (Steiger, et al. 2016. Metabolic Engineering 35. 95-104. doi:10.1016/j.ymben.2016.02.003) either under the control of the pAOX or the pGAP promoter. (e.g. using the plasmids pPM2d_pGAP and pPM2d_pAOX described in Example 6 as recipient plasmids). The plasmid containing a functional expression cassette containing cadA is transformed into strains GaT_pp_22 and GaT_pp_10 according to Example 6 resulting in GaT_pp_22+pGAP::CAD and GaT_pp_10+CAD.

The CAD producing strains (GaT_pp_22+CAD and GaT_pp_10+CAD) are then tested for itaconic acid production in fermentation studies, which are designed according to Examples 4 and 5. The production of itaconic acid during these cultivations is monitored by HPLC analysis (Blumhoff, et al 2013. Metabolic Engineering 19. 26-32. doi:10.1016/j.ymben.2013.05.003.; Steiger, et al. 2016. Metabolic Engineering 35. 95-104. doi:10.1016/j.ymben.2016.02.003) analogous to the sample preparations described in Example 3.

### Example 14 Construction of GaT_pp_22 Derivatives Secreting Porcine Carboxypeptidase B (CpB) or Human Serum Albumin (HSA)

P. pastoris CBS7435 variant and RuBisCO positive strains (denoted as GaT_pp_22 strains) were used as recipient strains. Strains expressing CpB and HSA in the background of GaT_pp_22 are constructed as described in Example 7 according to the procedure described for strain GaT_pp_10.

The final strains are denoted as GaT_pp_37 (CpB) with the genotype Δ(aox1)₁(das1)₂(das2)₃::(TDH3, PRK, PGK1)₁(RuBisCO, GroEL, GroES)₂(TKL1, TPI1)₃P_{AOX1}CpB and GaT_pp_38 (HSA) with the genotype Δ(aox1)₁(das1)₂(das2)₃::(TDH3, PRK, PGK1)₁(RuBisCO, GroEL, GroES)₂(TKL1, TPI1)₃P_{AOX1} EISA, respectively.

To test if the engineered GaT_pp_38 strains overexpressing HSA are able to produce heterologous proteins when carbon for biomass formation is solely provided by CO₂, bioreactor cultivations were performed. The set-up of these studies was designed accordingly to the set-ups described in example 4, 5 and 6.

For bioreactor cultivation of GaT_pp_38 strains, pre-cultures were prepared as follows.

Restreaks were made from cryo-stock solutions of GaT_pp_31 and GaT_pp_35 on YPD-plates and incubated for 48 h on 28° C. Single colonies were picked and used for inoculation of 400 mL of YPG medium. The pre-cultures were grown over night at 28° C. and 180 rpm. Optical density was determined and cell suspension was then transferred into 500 mL sterile centrifugation tubes and centrifuged (1500 g, 6 min). The pellet was washed with sterile dH₂O twice and then resuspended in 20 mL of sterile dH₂O. From this suspension, samples were taken and OD was determined. The volume needed for inoculation of 500 mL YNB medium supplemented with 0.5% methanol (starting OD=18.0 or 3.45 g*L⁻¹ CDW) was calculated.

After inoculation, the bioreactor cultivations were carried out in 1.4 L DASGIP reactors (Eppendorf, Germany) as described for example 4 with the alteration that the pH was adjusted using 5 M NaOH. The sampling procedure and maintenance of the methanol concentration in the reactors was also performed according to example 4.

The analytical procedure for detection of HSA by SDS-PAGE and silver ion staining was described in Example 7 and was applied here accordingly.

### Results Example 14

The cytosolic strains overexpressing HSA (GaT_pp_38) were cultivated as described above in two biological replicates. In the these cultivations, the cells still grew with biomass formation rate 0.012 and 0.008 g CDW L⁻¹h⁻¹′ respectively. In both cases HSA was produced in well detectable levels. FIG. 8 (lanes 6-13) shows that HSA is accumulated during the course of the bioreactor cultivation starting from undetectable levels on day 0 (d0) to well detectable levels on day 1 to day 3 (d1-3) in both biological replicates of GaT_pp_38. Due to the compact and globular form of HSA the apparent molecular mass detected by silver staining (here around 55 kDa) is smaller than the actual molecular mass of 66.4 kDa (congruent with unpublished previous data).

With this example it is shown that HSA, representing a model pharmaceutical protein, can be produced with strain GaT_pp_38, which harbors the cytosolic version of the synthetic Calvin cycle.

### REFERENCES CITED IN THE EXAMPLES SECTION

Blumhoff, Marzena L., Matthias G. Steiger, Diethard Mattanovich, and Michael Sauer. 2013. “Targeting Enzymes to the Right Compartment: Metabolic Engineering for Itaconic Acid Production by Aspergillus Niger.” Metabolic Engineering 19 (September). Elsevier: 26-32. doi:10.1016/j.ymben.2013.05.003.
Engler, Carola, Ramona Gruetzner, Romy Kandzia, and Sylvestre Marillonnet. 2009. “Golden Gate Shuffling: A One-Pot DNA Shuffling Method Based on Type IIs Restriction Enzymes.” PloS One 4 (5): e5553. doi:10.1371/journal.pone.0005553.
FOLK, J E, K A PIEZ, W R CARROLL, and J A GLADNER. 1960. “CarboxyPeptidase B. 4. Purification and Characterization of the Porcine Enzyme.” The Journal of Biological Chemistry 235 (August): 2272-77.
Gao, Yangbin, and Yunde Zhao. 2014. “Self-Processing of Ribozyme-Flanked RNAs into Guide RNAs in Vitro and in Vivo for CRISPR-Mediated Genome Editing.” Journal of Integrative Plant Biology 56 (4): 343-49. doi:10.1111/jipb.12152.
Jakočiūnas, Tadas, Michael K. Jensen, and Jay D. Keasling. 2015. “CRISPR/Cas9 Advances Engineering of Microbial Cell Factories.” Metabolic Engineering 34: 44-59. doi:10.1016/j.ymben.2015.12.003.
Küberl, Andreas, Jessica Schneider, Gerhard G. Thallinger, Ingund Anderl, Daniel Wibberg, Tanja Hajek, Sebastian Jaenicke, et al. 2011. “High-Quality Genome Sequence of Pichia Pastoris CBS7435.” Journal of Biotechnology 154 (4): 312-20. doi:10.1016/j.jbiotec.2011.04.014.
Sarkari, Parveen, and Michael Sauer. n.d. “An Efficient Tool for Metabolic Pathway Construction and Gene Integration for Aspergillus Niger.” Bioresource Technology. Elsevier Science. doi:10.1016/j.biortech.2017.05.004.

Steiger, Matthias G., Peter J. Punt, Arthur F. J. Ram, Diethard Mattanovich, and Michael Sauer. 2016. “Characterizing MttA as a Mitochondrial Cis-Aconitic Acid Transporter by Metabolic Engineering.” Metabolic Engineering 35 (May). Elsevier: 95-104. doi:10.1016/j.ymben.2016.02.003.
Valli, Minoska, Nadine E. Tatto, Armin Peymann, Clemens Gruber, Nils Landes, Heinz Ekker, Gerhard G. Thallinger, Diethard Mattanovich, Brigitte Gasser, and Alexandra B. Graf. 2016. “Curation of the Genome Annotation of Pichia Pastoris (Komagataella phaffii) CBS7435 from Gene Level to Protein Function.” FEMS Yeast Research 16 (6): 1-12. doi:10.1093/femsyr/fow051.
Weninger, Astrid, Anna-Maria Hatzl, Christian Schmid, Thomas Vogl, and Anton Glieder. 2016. “Combinatorial Optimization of CRISPR/Cas9 Expression Enables Precision Genome Engineering in the Methylotrophic Yeast Pichia Pastoris.” Journal of Biotechnology 235: 139-49. doi:10.1016/j.jbiotec.2016.03.027.

1. A yeast comprising a nucleotide sequence expression system expressing a synthetic Calvin cycle comprising heterologous genes of the synthetic Calvin cycle, wherein the heterologous genes comprise:
a) a gene encoding an enzyme from the class of the ribulose-bisphosphate carboxylases (EC number: 4.1.1.39) (RuBisCO gene); and
b) a gene encoding an enzyme from the class of the ribulose phosphate kinases (EC number: 2.7.1.19) (PRK gene);
wherein each of said RuBisCO gene and said PRK gene is fused with a nucleotide sequence encoding a peroxisomal targeting signal (PTS).

2. The yeast of claim 1, further comprising a heterologous expression construct expressing a gene of interest (GOI).

3. (canceled)

4. The yeast of claim 1, wherein the yeast comprises one or more endogenous genes to complete the synthetic Calvin cycle.

5. The yeast of claim 1, wherein the synthetic Calvin cycle comprises one or more further heterologous genes selected from the group consisting of:
a) a gene encoding an enzyme from the class of the phosphoglycerate kinases (EC number: 2.7.2.3) (PGK1 gene), and/or
b) a gene encoding an enzyme from the class of the glyceraldehyde-3-phosphate dehydrogenases (EC number 1.2.1.12) (TDH3 gene); and/or
c) a gene encoding an enzyme from the class of the triose-phosphate isomerases (EC number 5.3.1.1) (TPI1 gene); and
d) a gene encoding an enzyme from the class of the transketolases (EC number 2.2.1.1) (TKL1 gene),
wherein none, one or more, or all of said PGK1, TDH3, TPI1, and TKL1 genes are fused with a nucleotide sequence encoding a PTS.

6. The yeast of claim 5, wherein the synthetic Calvin cycle comprises the following heterologous genes: said RuBisCO gene, said PRK gene, said PGK1 gene, said TDH3 gene, said TPI1 gene, and said TKL1 gene.

7. The yeast of claim 5, wherein:
a) said RuBisCO gene is of Thiobacillus denitrificans origin; and/or
b) said PRK gene is of Spinacia oleracea origin; and/or
c) said PGK1 gene is of Ogataea polymorpha origin; and/or
d) said TDH3 gene is of Ogataea polymorpha origin; and/or
e) said TPI1 gene is of Ogataea parapolymorpha origin; and/or; and/or
f) said TKL1 gene is of Ogataea parapolymorpha origin.

8. The yeast of claim 1, wherein the yeast comprises further heterologous genes expressing one or more molecular chaperones in the cytosol of said yeast, which chaperones assist the covalent folding and/or assembly of at least one of said enzymes.

9. The yeast of claim 8, wherein said chaperones comprise:
a) a GroEL gene of Escherichia coli origin; and
b) a GroES gene of Escherichia coli origin.

10. The yeast of claim 1, wherein one or more of said heterologous genes of the synthetic Calvin cycle are codon-optimized for expression in said yeast.

11. The yeast of claim 1, wherein the yeast is of a genus selected from the group consisting of Pichia, Komagataella, Hansenula, Ogataea, Candida, and Torulopsis.

12. A method of culturing the yeast of claim 1 in a cell culture, comprising the step of culturing the yeast in the growing phase using gaseous carbon dioxide and/or dissolved CO₃²⁻ and/or HCO₃⁻ compounds as a carbon source, thereby obtaining accumulated yeast biomass in the cell culture.

13. The method of claim 12, wherein the yeast incorporates one or more heterologous genes operably linked to a promoter which is inducible by methanol, and wherein said growing phase starts upon the further step of adding methanol to the culture medium.

14. The method of claim 13, further comprising the step of culturing said accumulated yeast biomass in a production phase using a carbon source to produce a protein of interest (POI) from said heterologous genes or a metabolite from the enzymatic reaction of the POI.

15. A method of producing an organic product in the yeast of claim 1, wherein at least 20% of the product's total organic carbon is from a carbon source which is gaseous carbon dioxide and/or dissolved CO₃²⁻ and/or HCO₃⁻ compounds.

16. (canceled)

17. The yeast of claim 7, wherein:
a) the RuBisCO gene comprises the nucleotide sequence of SEQ ID NO:37, or a functionally active variant thereof with at least 90% sequence identity expressing a ribulose-bisphosphate carboxylase; and/or
b) the PRK gene comprises the nucleotide sequence of SEQ ID NO:38, or a functionally active variant thereof with at least 90% sequence identity expressing a ribulose phosphate kinase; and/or
c) the PGK1 gene the comprises the nucleotide sequence of SEQ ID NO:39, or a functionally active variant thereof with at least 90% sequence identity expressing a phosphoglycerate kinase; and/or
d) the TDH3 gene comprises the nucleotide sequence of SEQ ID NO:40, or a functionally active variant thereof with at least 90% sequence identity expressing a glyceraldehyde-3-phosphate dehydrogenase; and/or
e) said TPI1 gene comprises the nucleotide sequence of SEQ ID NO:41, or a functionally active variant thereof with at least 90% sequence identity expressing a triose-phosphate isomerase; and/or; and/or
f) said TKL1 gene comprises the nucleotide sequence of SEQ ID NO:42, or a functionally active variant thereof with at least 90% sequence identity expressing a transketolase.

18. The yeast of claim 9, wherein:
a) said GroEL gene comprises the nucleotide sequence of SEQ ID NO:43, or a functionally active variant thereof with at least 90% sequence identity expressing a molecular chaperone; and
b) said GroES gene comprises the nucleotide sequence of SEQ ID NO:8, or a functionally active variant thereof with at least 90% sequence identity expressing a molecular chaperone.

19. The yeast of claim 11, wherein the strain is selected from the group consisting of Pichia pastoris, Komagataella pastoris, Komagateaella phaffii, and Komagateaella pseudopastoris
